SEQUENCE LISTING (80 sequences)

SEQ ID NO: 1 - protein, 298 aa, Arabidopsis thaliana
MAEEKKAPISVWTTVKPFVNGGASGMLATCVIQPIDMIKVRIQLGQGSAASITTNMLKNEGVGAFYKGLSAGLLRQATYTTARLGSFKLLTAKAIESNDGKPLPLYQKALCGLTAGAIGACVGSPADLALIRMQADNTLPLAQRRNYTNAFHALTRISADEGVLALWKGCGPTVVRAMALNMGMLASYDQSAEYMRDNLGFGEMSTVVGASAVSGFCAAACSLPFDFVKTQIQKMQPDAQGKYPYTGSLDCAMKTLKEGGPLKFYSGFPVYCVRIAPHVMMTWIFLNQITKFQKKIGM

SEQ ID NO: 2 - protein, 313 aa, Arabidopsis thaliana
MGLKGFAEGGIASIVAGCSTHPLDLIKVRMQLQGESAPIQTNLRPALAFQTSTTVNAPPLRVGVIGVGSRLIREEGMRALFSGVSATVLRQTLYSTTRMGLYDIIKGEWTDPETKTMPLMKKIGAGAIAGAIGAAVGNPADVAMVRMQADGRLPLTDRRNYKSVLDAITQMIRGEGVTSLWRGSSLTINRAMLVTSSQLASYDSVKETILEKGLLKDGLGTHVSASFAAGFVASVASNPVDVIKTRVMNMKVVAGVAPPYKGAVDCALKTVKAEGIMSLYKGFIPTVSRQAPFTVVLFVTLEQVKKLFKDYDF

SEQ ID NO: 3 - protein, 313 aa, Arabidopsis thaliana
MGVKSFVEGGIASVIAGCSTHPLDLIKVRLQLHGEAPSTTTVTLLRPALAFPNSSPAAFLETTSSVPKVGPISLGINIVKSEGAAALFSGVSATLLRQTLYSTTRMGLYEVLKNKWTDPESGKLNLSRKIGAGLVAGGIGAAVGNPADVAMVRMQADGRLPLAQRRNYAGVGDAIRSMVKGEGVTSLWRGSALTINRAMIVTAAQLASYDQFKEGILENGVMNDGLGTHVVASFAAGFVASVASNPVDVIKTRVMNMKVGAYDGAWDCAVKTVKAEGAMALYKGFVPTVCRQGPFTVVLFVTLEQVRKLLRDF

SEQ ID NO: 4 - protein, 337 aa, Arabidopsis thaliana
MGFKPFLEGGIAAIIAGALTHPLDLIKVRMQLQGEHSFSLDQNPNPNLSLDHNLPVKPYRPVFALDSLIGSISLLPLHIHAPSSSTRSVMTPFAVGAHIVKTEGPAALFSGVSATILRQMLYSATRMGIYDFLKRRWTDQLTGNFPLVTKITAGLIAGAVGSVVGNPADVAMVRMQADGSLPLNRRRNYKSVVDAIDRIARQEGVSSLWRGSWLTVNRAMIVTASQLATYDHVKEILVAGGRGTPGGIGTHVAASFAAGIVAAVASNPIDVVKTRMMNADKEIYGGPLDCAVKMVAEEGPMALYKGLVPTATRQGPFTMILFLTLEQVRGLLKDVKF

SEQ ID NO: 5 - DNA, 11200 bp, Artificial Sequence
  Synthetic construct pYTEN-10
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatggcggaagagaagaaagctccaatcagtgtctggactaccgtgaagcccttcgtcaatggcggtgcctctggtatgctcgctacttgcgttatccagccgatcgacatgattaaggtgaggattcaacttggtcagggatctgcagccagtataaccaccaacatgcttaagaatgagggcgttggtgccttctacaagggattatctgctgggttgctgaggcaagcaacttacacgacagcccgtcttggatcattcaagttgctgactgcaaaggcaattgagtctaatgatggaaagcctctaccgctgtatcagaaggcactatgtggtctgacagctggtgctattggtgcttgcgtcggtagtccagccgatctagcacttatcagaatgcaggccgataatactttgccgctagctcagcgcaggaattataccaatgctttccatgcgcttacccgtattagcgctgatgagggagttttagcactttggaaagggtgtgggcccactgtggtcagagctatggctttgaacatgggaatgcttgcatcttatgatcaaagtgctgaatacatgagagataatcttggtttcggggagatgtctacggtcgtaggagcaagtgctgtttctgggttctgcgctgcggcttgcagtctgccatttgactttgtcaaaactcagattcagaaaatgcaaccggatgctcaaggaaagtatccatacacaggttcgctcgattgtgcgatgaaaaccttaaaagaaggaggacctctgaaattttactcgggtttcccagtttactgtgtcaggattgcccctcacgtcatgatgacatggatcttcctaaaccagattacgaaatttcaaaagaagattggtatgtgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 6 - DNA, 11244 bp, Artificial Sequence
  Synthetic construct pYTEN-11
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgggtctaaagggttttgctgaaggaggaatagcttcgattgttgcgggttgttcgacccacccgcttgatctaatcaaggtccgaatgcaacttcaaggcgaatcagctccgattcaaaccaatctccgaccagctcttgcttttcagacttcgaccaccgtcaacgcgcctcctctacgtgttggtgtaatcggagtcggatctcgtttaataagagaagaaggcatgcgtgctctgttttccggcgtctccgccaccgttcttcgtcaaactctgtattcaacgactcgtatgggtttatacgacatcatcaaaggagaatggaccgacccggaaacaaaaacgatgcctttaatgaaaaaaatcggtgccggagccatcgccggagcaatcggagccgccgttgggaatcctgctgacgtggcgatggtgaggatgcaagccgatggtcgtttaccgttgactgatagaagaaactacaaaagcgttttagacgcgatcacgcaaatgattcgcggtgaaggcgttacgtcgttgtggagaggatcgtctttgacgataaacagagcaatgcttgtgacgtcatcgcagttggcttcgtatgattctgttaaagagacgattttggagaaagggttgttgaaagatgggcttgggactcatgtgtcggcgagtttcgcggcggggtttgttgcgagcgttgcgagtaatcctgttgatgtgattaagacgagagtgatgaatatgaaggtggtggctggagttgctccgccgtataaaggagcggttgattgtgctttgaaaacggtgaaagcggaagggattatgtctttgtataaaggttttatcccgacggtttcgagacaagcaccgttcacggtggttttgtttgttacgcttgaacaagttaagaagttgttcaaggactatgacttttgcgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 7 - DNA, 11161 bp, Artificial Sequence
  Synthetic construct pYTEN-12
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgggagtcaaaagtttcgttgaaggtgggattgcctctgtaatcgccggttgctctactcaccctctcgatctaatcaaggttcgtcttcagcttcacggtgaagcaccttccaccaccaccgtcactctcctccgtccagctctcgctttccccaattcttctcctgcagctttcctggaaacgacttcttcagtccccaaagtaggaccgatctcactcggaatcaacatagtcaaatcggaaggcgccgccgcgttattctcaggagtctccgctacacttctccgtcagacgttatattccaccaccaggatgggtctatacgaagtgcttaagaacaaatggactgatcctgagtcagggaagttgaatctgagtaggaagatcggtgcagggctagtcgctggtggaatcggagccgccgttggaaatccagctgacgtggcgatggttaggatgcaagctgacgggaggttacctttagcgcaacgtcgtaactacgccggagtaggagacgcaatcaggagcatggttaagggagaaggcgtaacgagcttgtggcgaggctcggcgttgacgattaaccgagcgatgattgtgacggcggctcagctagcgtcttacgatcagttcaaggaagggatattggagaatgatgtgataaagacgagagtgatgaatatgaaggtgggagcgtacgacggcgcgtgggattgtgcggtgaagacggttaaagcggaaggagccatggctctttataaaggctttgttcctacagtttgtaggcaaggtcctttcactgttgttctcttcgttacgttggagcaagttaggaagctgcttcgagatttttgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 8 - DNA, 11317 bp, Artificial Sequence
  Synthetic construct pYTEN-13
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgggcttcaaaccatttcttgaaggtggcatcgccgcaatcatcgccggagctctgactcacccattagacctcatcaaagtccgtatgcagcttcaaggcgaacattctttctcgctcgaccaaaaccctaaccctaatcttagccttgatcataatcttcccgtgaaaccttaccgacccgttttcgctcttgactctctcatcggcagcatttccttattacccttacacattcacgcgccgtcttcttccacgcgctccgtcatgacccctttcgccgtaggggcacacattgtcaaaaccgaaggacccgccgctctcttctccggcgtctccgccaccatcctccgtcagatgctttactctgccacccgtatgggtatatacgactttcttaaacgaaggtggactgatcaactcaccggtaacttcccattggtaaccaagatcacagctggactcatcgcaggagccgttggatcagtcgtcgggaatcctgctgacgtggcgatggtgagaatgcaagccgacggaagcttgccgttaaaccgtcgcagaaactacaagagcgtcgtggacgcgatcgacaggatcgcaaggcaagaaggcgtttcaagtttatggcgtggctcatggctaaccgtgaaccgtgccatgatcgtgacagcttctcagctcgctacgtatgatcacgtcaaagaaatcttggtagctggtggccgtggaacgccgggagggataggaacgcatgtagcggcgagttttgcggcggggatcgtcgcggcggtggcgtcgaatcccatcgacgttgtgaagacgaggatgatgaatgcggataaggagatttacggtggaccgttggattgtgcggtgaagatggtggcagaggaaggaccaatggctttgtacaaagggcttgttccgacggcgacgaggcaaggaccgttcacgatgatcttgttccttaccttggaacaagttcgtggtctgttaaaagacgttaaattttgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 9 - DNA, 10926 bp, Artificial Sequence
  Synthetic construct pYTEN-14
tccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatcacaagtttgtacaaaaaagcaggctccgcggccgcccccttcaccatggcggaagagaagaaagctccaatcagtgtctggactaccgtgaagcccttcgtcaatggcggtgcctctggtatgctcgctacttgcgttatccagccgatcgacatgattaaggtgaggattcaacttggtcagggatctgcagccagtataaccaccaacatgcttaagaatgagggcgttggtgccttctacaagggattatctgctgggttgctgaggcaagcaacttacacgacagcccgtcttggatcattcaagttgctgactgcaaaggcaattgagtctaatgatggaaagcctctaccgctgtatcagaaggcactatgtggtctgacagctggtgctattggtgcttgcgtcggtagtccagccgatctagcacttatcagaatgcaggccgataatactttgccgctagctcagcgcaggaattataccaatgctttccatgcgcttacccgtattagcgctgatgagggagttttagcactttggaaagggtgtgggcccactgtggtcagagctatggctttgaacatgggaatgcttgcatcttatgatcaaagtgctgaatacatgagagataatcttggtttcggggagatgtctacggtcgtaggagcaagtgctgtttctgggttctgcgctgcggcttgcagtctgccatttgactttgtcaaaactcagattcagaaaatgcaaccggatgctcaaggaaagtatccatacacaggttcgctcgattgtgcgatgaaaaccttaaaagaaggaggacctctgaaattttactcgggtttcccagtttactgtgtcaggattgcccctcacgtcatgatgacatggatcttcctaaaccagattacgaaatttcaaaagaagattggtatgtgaaagggtgggcgcgccgacccagctttcttgtacaaagtggtgcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaat

SEQ ID NO: 10 - DNA, 10970 bp, Artificial Sequence
  Synthetic construct pYTEN-15
aattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatcacaagtttgtacaaaaaagcaggctccgcggccgcccccttcaccatgggtctaaagggttttgctgaaggaggaatagcttcgattgttgcgggttgttcgacccacccgcttgatctaatcaaggtccgaatgcaacttcaaggcgaatcagctccgattcaaaccaatctccgaccagctcttgcttttcagacttcgaccaccgtcaacgcgcctcctctacgtgttggtgtaatcggagtcggatctcgtttaataagagaagaaggcatgcgtgctctgttttccggcgtctccgccaccgttcttcgtcaaactctgtattcaacgactcgtatgggtttatacgacatcatcaaaggagaatggaccgacccggaaacaaaaacgatgcctttaatgaaaaaaatcggtgccggagccatcgccggagcaatcggagccgccgttgggaatcctgctgacgtggcgatggtgaggatgcaagccgatggtcgtttaccgttgactgatagaagaaactacaaaagcgttttagacgcgatcacgcaaatgattcgcggtgaaggcgttacgtcgttgtggagaggatcgtctttgacgataaacagagcaatgcttgtgacgtcatcgcagttggcttcgtatgattctgttaaagagacgattttggagaaagggttgttgaaagatgggcttgggactcatgtgtcggcgagtttcgcggcggggtttgttgcgagcgttgcgagtaatcctgttgatgtgattaagacgagagtgatgaatatgaaggtggtggctggagttgctccgccgtataaaggagcggttgattgtgctttgaaaacggtgaaagcggaagggattatgtctttgtataaaggttttatcccgacggtttcgagacaagcaccgttcacggtggttttgtttgttacgcttgaacaagttaagaagttgttcaaggactatgacttttgaagggtgggcgcgccgacccagctttcttgtacaaagtggtgcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcag

SEQ ID NO: 11 - DNA, 10887 bp, Artificial Sequence
  Synthetic construct pYTEN-16
aattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatcacaagtttgtacaaaaaagcaggctccgcggccgcccccttcaccatgggagtcaaaagtttcgttgaaggtgggattgcctctgtaatcgccggttgctctactcaccctctcgatctaatcaaggttcgtcttcagcttcacggtgaagcaccttccaccaccaccgtcactctcctccgtccagctctcgctttccccaattcttctcctgcagctttcctggaaacgacttcttcagtccccaaagtaggaccgatctcactcggaatcaacatagtcaaatcggaaggcgccgccgcgttattctcaggagtctccgctacacttctccgtcagacgttatattccaccaccaggatgggtctatacgaagtgcttaagaacaaatggactgatcctgagtcagggaagttgaatctgagtaggaagatcggtgcagggctagtcgctggtggaatcggagccgccgttggaaatccagctgacgtggcgatggttaggatgcaagctgacgggaggttacctttagcgcaacgtcgtaactacgccggagtaggagacgcaatcaggagcatggttaagggagaaggcgtaacgagcttgtggcgaggctcggcgttgacgattaaccgagcgatgattgtgacggcggctcagctagcgtcttacgatcagttcaaggaagggatattggagaatgatgtgataaagacgagagtgatgaatatgaaggtgggagcgtacgacggcgcgtgggattgtgcggtgaagacggttaaagcggaaggagccatggctctttataaaggctttgttcctacagtttgtaggcaaggtcctttcactgttgttctcttcgttacgttggagcaagttaggaagctgcttcgagatttttgaaagggtgggcgcgccgacccagctttcttgtacaaagtggtgcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcag

SEQ ID NO: 12 - DNA, 11043 bp, Artificial Sequence
  Synthetic construct pYTEN-17
aattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatcacaagtttgtacaaaaaagcaggctccgcggccgcccccttcaccatgggcttcaaaccatttcttgaaggtggcatcgccgcaatcatcgccggagctctgactcacccattagacctcatcaaagtccgtatgcagcttcaaggcgaacattctttctcgctcgaccaaaaccctaaccctaatcttagccttgatcataatcttcccgtgaaaccttaccgacccgttttcgctcttgactctctcatcggcagcatttccttattacccttacacattcacgcgccgtcttcttccacgcgctccgtcatgacccctttcgccgtaggggcacacattgtcaaaaccgaaggacccgccgctctcttctccggcgtctccgccaccatcctccgtcagatgctttactctgccacccgtatgggtatatacgactttcttaaacgaaggtggactgatcaactcaccggtaacttcccattggtaaccaagatcacagctggactcatcgcaggagccgttggatcagtcgtcgggaatcctgctgacgtggcgatggtgagaatgcaagccgacggaagcttgccgttaaaccgtcgcagaaactacaagagcgtcgtggacgcgatcgacaggatcgcaaggcaagaaggcgtttcaagtttatggcgtggctcatggctaaccgtgaaccgtgccatgatcgtgacagcttctcagctcgctacgtatgatcacgtcaaagaaatcttggtagctggtggccgtggaacgccgggagggataggaacgcatgtagcggcgagttttgcggcggggatcgtcgcggcggtggcgtcgaatcccatcgacgttgtgaagacgaggatgatgaatgcggataaggagatttacggtggaccgttggattgtgcggtgaagatggtggcagaggaaggaccaatggctttgtacaaagggcttgttccgacggcgacgaggcaaggaccgttcacgatgatcttgttccttaccttggaacaagttcgtggtctgttaaaagacgttaaattttgaaagggtgggcgcgccgacccagctttcttgtacaaagtggtgcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcag

SEQ ID NO: 13 - DNA, 6145 bp, Artificial Sequence
  Synthetic construct pYTEN-18
tttaatgtaatcactcaaataaataatatgaatctgagctatactacgagaacttctggattcagcaagaactagcagcaatcagaacccaatagcatagcaacaaaccgaacaatcaaccatatattaggagacggtagatagaaccacgttaacattaagggggtgtttgaatgcactgaaactaattgttagttggctaaaaattgttagttgaattagctagctaacaaataactacctcactattaactaattttccaaaaatagctaatagttcaactattagctatggtgtttggatgttttaactaattttagccactaactattagttttagtgcattcaaacacctcctaagtaagaaacggtagatagccagtacctgcaggcaaatattaggagacaactgaaagacagaacataatgagcacaggctttaatttcaaacatcaaacttattcatgatttgtcatagttctgggtagtacgcacacacaacacaaccggtccattattaaaccaacactgacacgactcatgacacgaacagcagatactttgacaacctccatatggagagagggcaccagacgacgcaggcacatcggcagcttaaacgacccatgactcgagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtagactgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgatcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattggggtttaaaccacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcggttaattaaatggcggacgcgaagcagcagcagcagcagcagcagccacagcaggcggcagcggcagccaccggcgtgtggaagacggtcaagcccttcgttaacggcggggcctctgggatgctcgcgacctgcgtcatccagcctatcgacatggtcaaggtgaggatccagttgggtgagggctctgctggtcaggtcacaaggaacatgcttgcaaatgagggtgtccgttctttctacaagggtttgtccgccggattgctgaggcaagcgacgtacacgactgctcgtcttggatcctttagggttctaactaacaaagcagttgaaaagaatgaagggaagccattgcctctatttcagaaagcttttattggtctgactgctggtgcaattggtgcttgtgttggtagtcctgctgatctggcactcattagaatgcaagccgattcgaccctgccagttgcacaacgacgcaactataagaatgctttccatgcactctaccgtatcagtggtgatgagggagtccttgcgctttggaagggtgcaggtccaactgtggtgagagctatggcactcaatatgggtatgcttgcttcctatgaccagagtgtcgagctatttagggacaaatttggcgcaggagaaatttctactgttgttggagccagcgctgtttctggattctttgcctcagcatgcagtttgccctttgactatgtgaagacacagattcagaagatgcaacctgatgcgaatggcaagtacccatacacagggtctttggactgtgctgtgaagaccttcaagagcggtggcccattcaagttctacactggtttcccggtgtactgcgtcaggattgcaccccatgtcatgatgacctggatattcttgaatcagatccagaagtttgagaagaagatcggcatatagggcgcgccgctcaacggctatgctatgcaacttcattgtctttcggatcggagagggtgtacgtacgtggattgattgatgctgcgagatgcatgtgtgtcttttgtttcacgttgcattgcataggcaagtcgagatgatgagtgggcgttgtacactaagatgaaccatgtttgtgcaatagtggtggtttttgtttcctgctggttaattgttgatatccattaatttgtttttcttcaaaaaaaaaaaaaaaaatgataagctgtcaaacatgacctcaggatgaagctt

SEQ ID NO: 14 - DNA, 6896 bp, Artificial Sequence
  Synthetic construct pYTEN-19
tttaatgtaatcactcaaataaataatatgaatctgagctatactacgagaacttctggattcagcaagaactagcagcaatcagaacccaatagcatagcaacaaaccgaacaatcaaccatatattaggagacggtagatagaaccacgttaacattaagggggtgtttgaatgcactgaaactaattgttagttggctaaaaattgttagttgaattagctagctaacaaataactacctcactattaactaattttccaaaaatagctaatagttcaactattagctatggtgtttggatgttttaactaattttagccactaactattagttttagtgcattcaaacacctcctaagtaagaaacggtagatagccagtacctgcaggcaaatattaggagacaactgaaagacagaacataatgagcacaggctttaatttcaaacatcaaacttattcatgatttgtcatagttctgggtagtacgcacacacaacacaaccggtccattattaaaccaacactgacacgactcatgacacgaacagcagatactttgacaacctccatatggagagagggcaccagacgacgcaggcacatcggcagcttaaacgacccatgactcgagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtagactgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgatcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattggggtttaaaccacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcggttaattaaatggcggacgcgaagcagcagcagcagcagcagcagccacagcaggcggcagcggcagccaccggcgtgtggaagacggtcaagcccttcgttaacggcggggcctctgggatgctcgcgacctgcgtcatccagcctatcgacatggtcaaggtgaggatccagttgggtgagggctctgctggtcaggtcacaaggaacatgcttgcaaatgagggtgtccgttctttctacaagggtttgtccgccggattgctgaggcaagcgacgtacacgactgctcgtcttggatcctttagggttctaactaacaaagcagttgaaaagaatgaagggaagccattgcctctatttcagaaagcttttattggtctgactgctggtgcaattggtgcttgtgttggtagtcctgctgatctggcactcattagaatgcaagccgattcgaccctgccagttgcacaacgacgcaactataagaatgctttccatgcactctaccgtatcagtggtgatgagggagtccttgcgctttggaagggtgcaggtccaactgtggtgagagctatggcactcaatatgggtatgcttgcttcctatgaccagagtgtcgagctatttagggacaaatttggcgcaggagaaatttctactgttgttggagccagcgctgtttctggattctttgcctcagcatgcagtttgccctttgactatgtgaagacacagattcagaagatgcaacctgatgcgaatggcaagtacccatacacagggtctttggactgtgctgtgaagaccttcaagagcggtggcccattcaagttctacactggtttcccggtgtactgcgtcaggattgcaccccatgtcatgatgacctggatattcttgaatcagatccagaagtttgagaagaagatcggcatatagggcgcgccgccaaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaacgaattctgataagctgtcaaacatgacctcaggatgaagctt

SEQ ID NO: 15 - DNA, 6199 bp, Artificial Sequence
  Synthetic construct pYTEN-20
tttaatgtaatcactcaaataaataatatgaatctgagctatactacgagaacttctggattcagcaagaactagcagcaatcagaacccaatagcatagcaacaaaccgaacaatcaaccatatattaggagacggtagatagaaccacgttaacattaagggggtgtttgaatgcactgaaactaattgttagttggctaaaaattgttagttgaattagctagctaacaaataactacctcactattaactaattttccaaaaatagctaatagttcaactattagctatggtgtttggatgttttaactaattttagccactaactattagttttagtgcattcaaacacctcctaagtaagaaacggtagatagccagtacctgcaggcaaatattaggagacaactgaaagacagaacataatgagcacaggctttaatttcaaacatcaaacttattcatgatttgtcatagttctgggtagtacgcacacacaacacaaccggtccattattaaaccaacactgacacgactcatgacacgaacagcagatactttgacaacctccatatggagagagggcaccagacgacgcaggcacatcggcagcttaaacgacccatgactcgagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtagactgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgatcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattggggtttaaaccacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcggttaattaaatgggtgtcaagggattcgtcgagggcggcatcgcctccatcgtggccggctgctccacccacccgctggacctcatcaaggtccgcatgcagctgcagggggaggcggcggccgccgtggcgccgcagccggcgctgcgcccggcgctcgccttccacgccgggggccacgccgtcgcgctcccgcaccaccaccaccacgacatcccggcgccaccgcggaagcccgggccgctggccgtcggcgcgcagatcctgcggtccgagggcgccgcgggcctcttctcgggggtgtccgccaccatgctgcggcagacgctctactccaccacgcggatggggctgtacgacatcctcaagaccaggtgggcccgggagaacggcggcgtcctcccgctgcaccgcaagatcttggccggcctcgtcgccggcggcgtcggcgccgccgtgggcaacccggccgacgtggccatggtgcggatgcaggcggacgggcgcctgcccctcgcggagcgccggaactaccgcggcgtcggcgacgccatcggccggatggcgcgcgacgagggcgtgcgcagcctctggcgcgggtcctcgctcaccgtcaaccgcgccatgatcgtcacggcgtcgcagctggccacgtacgaccaggccaaggaggccatcctggcgcgccgcggcccgggcgccgacgggctcgccacgcacgtggccgccagcttcaccgccggcatcgtcgccgccgccgcgtccaaccccgtcgacgtcgtcaagacgaggatgatgaacatgaaggtggcgcccggcgcgcccccgccctacgccggcgccgtcgactgcgccctcaagaccgtcaggtcggagggacccatggcgctgtacaaggggttcatccccaccgtcatgcgccaggggcccttcaccgttgtgctcttcgtcacgctcgagcaggtgcgcaaggtcttcaagggcgtcgagttctgaggcgcgccgctcaacggctatgctatgcaacttcattgtctttcggatcggagagggtgtacgtacgtggattgattgatgctgcgagatgcatgtgtgtcttttgtttcacgttgcattgcataggcaagtcgagatgatgagtgggcgttgtacactaagatgaaccatgtttgtgcaatagtggtggtttttgtttcctgctggttaattgttgatatccattaatttgtttttcttcaaaaaaaaaaaaaaaaatgataagctgtcaaacatgacctcaggatgaagctt

SEQ ID NO: 16 - DNA, 6950 bp, Artificial Sequence
  Synthetic construct pYTEN-21
tttaatgtaatcactcaaataaataatatgaatctgagctatactacgagaacttctggattcagcaagaactagcagcaatcagaacccaatagcatagcaacaaaccgaacaatcaaccatatattaggagacggtagatagaaccacgttaacattaagggggtgtttgaatgcactgaaactaattgttagttggctaaaaattgttagttgaattagctagctaacaaataactacctcactattaactaattttccaaaaatagctaatagttcaactattagctatggtgtttggatgttttaactaattttagccactaactattagttttagtgcattcaaacacctcctaagtaagaaacggtagatagccagtacctgcaggcaaatattaggagacaactgaaagacagaacataatgagcacaggctttaatttcaaacatcaaacttattcatgatttgtcatagttctgggtagtacgcacacacaacacaaccggtccattattaaaccaacactgacacgactcatgacacgaacagcagatactttgacaacctccatatggagagagggcaccagacgacgcaggcacatcggcagcttaaacgacccatgactcgagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtagactgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgatcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattggggtttaaaccacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcggttaattaaatgggtgtcaagggattcgtcgagggcggcatcgcctccatcgtggccggctgctccacccacccgctggacctcatcaaggtccgcatgcagctgcagggggaggcggcggccgccgtggcgccgcagccggcgctgcgcccggcgctcgccttccacgccgggggccacgccgtcgcgctcccgcaccaccaccaccacgacatcccggcgccaccgcggaagcccgggccgctggccgtcggcgcgcagatcctgcggtccgagggcgccgcgggcctcttctcgggggtgtccgccaccatgctgcggcagacgctctactccaccacgcggatggggctgtacgacatcctcaagaccaggtgggcccgggagaacggcggcgtcctcccgctgcaccgcaagatcttggccggcctcgtcgccggcggcgtcggcgccgccgtgggcaacccggccgacgtggccatggtgcggatgcaggcggacgggcgcctgcccctcgcggagcgccggaactaccgcggcgtcggcgacgccatcggccggatggcgcgcgacgagggcgtgcgcagcctctggcgcgggtcctcgctcaccgtcaaccgcgccatgatcgtcacggcgtcgcagctggccacgtacgaccaggccaaggaggccatcctggcgcgccgcggcccgggcgccgacgggctcgccacgcacgtggccgccagcttcaccgccggcatcgtcgccgccgccgcgtccaaccccgtcgacgtcgtcaagacgaggatgatgaacatgaaggtggcgcccggcgcgcccccgccctacgccggcgccgtcgactgcgccctcaagaccgtcaggtcggagggacccatggcgctgtacaaggggttcatccccaccgtcatgcgccaggggcccttcaccgttgtgctcttcgtcacgctcgagcaggtgcgcaaggtcttcaagggcgtcgagttctgaggcgcgccgccaaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaacgaattctgataagctgtcaaacatgacctcaggatgaagctt

SEQ ID NO: 17 - DNA, 14587 bp, Artificial Sequence
  Synthetic construct pYTEN-22
gcgtataatggactattgtgtgctgataaggagaacataagcgcagaacaatatgtatctattccggtgttgtgttcctttgttattctgctattatgttctcttatagtgtgacgaaagcagcataattaatcgtcacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagagaccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcaatggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaaccttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcgggttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtccggtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaggggatcctctagacgcagaaaggcccacccgaaggtgagccagtgtgattacatttgcggcctaactgtggccagtccagttacgctggagtcactagtgcggccgcgacaacttgtctagggcccaatggcccgggactggcgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggtaccaaaatgggagacgagaataagaccaagtcccctgcttccggcgtgtggtccaccatcaagcctttcgtcaatggcggagcctccggcatgctcgccacctgcgtcattcaacccatcgatatgatcaaggtgaggattcaacttgggcaaggatcagctgcgcaggtcacttccaccatgcttaagaatgagggtgttgctgccttctataagggtctatctgctggattactcaggcaggctacatacaccactgcccgtcttggatcatttaaaatcttgacggccaaagctattgaggctaatgatgggaagcccctgccactgtatcagaaagctctgtgtgggctgactgctggtgctatcggagcaagtgttggtagtccagcagacttggcactcattcggatgcaggctgatgcaacattacctgctgctcagcgccgaaattacacaaatgccttccatgcactctatcgaattactgcagatgaaggggttttggcgctttggaaaggtgctgggcctactgttgtaagagccatggcattgaacatgggcatgcttgcatcttatgatcaaagtgttgagttcttcagggattctgttggtcttggtgaaggtgctactgtgctaggtgcaagttctgtttctggatttttcgcagcagcttgcagtttaccatttgactatgtcaagacccagatccagaagatgcaacctgatgctgatgggaaatatccatacactggctccgttgattgtgctgtcaaaaccttcaaagcaggaggaccattcaaattttacaccggattccctgtctattgtgttaggattgctcctcatgtgatgatgacatggattttcctgaaccagatacagaaattgcagaaatcctacgggttgtagtctagagcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaaggcgcgccagtcccgggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtatttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtcgagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtatgacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 18 - DNA, 14635 bp, Artificial Sequence
  Synthetic construct pYTEN-23
gcgtataatggactattgtgtgctgataaggagaacataagcgcagaacaatatgtatctattccggtgttgtgttcctttgttattctgctattatgttctcttatagtgtgacgaaagcagcataattaatcgtcacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagagaccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcaatggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaaccttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcgggttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtccggtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaggggatcctctagacgcagaaaggcccacccgaaggtgagccagtgtgattacatttgcggcctaactgtggccagtccagttacgctggagtcactagtgcggccgcgacaacttgtctagggcccaatggcccgggactggcgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggtaccaaaatgggcgtcaaaggttttgtcgaaggaggcatcgcttccatcattgcaggatgttccacacacccacttgatctcatcaaggtccgcatgcagcttcagggcgaaaacaatttgcccaaaccggttcaaaatctccgacccgcactcgccttccaaaccggttcgaccgtccacgtggcagcggctattccgcagacccgcgtgggtcccatcgcggttggggttcgcctcgtccagcaagaaggccttgcggccttgttctccggcgtctccgccactgtcctccgccagacgctctactccaccacccgtatgggcctctacgacgtcctcaagaccaagtggaccgactccgtcaccggcaccatgccgctcagccgcaagatcgaggccggtctcatcgccggtggcatcggcgccgccgtggggaaccccgccgacgtggccatggtccgaatgcaggcagacgggcgcctccctccggcacagcggcgcaactacaagtccgtcgtggacgccatcacgcgaatggcgaagcaagaaggcgtcactagcctttggagaggctcatcgcttacggtgaaccgcgccatgctcgtgacggcgtcgcagctcgcgtcgtacgaccagttcaaagaaacgatcttggagaacggcatgatgcgcgacgggctcgggacccatgtcacggcgagcttcgcggcggggttcgtggcggcggtggcgtcgaaccccgtcgacgtgatcaagacgagggtgatgaacatgagggtggagcccggggcgacgccgccctacgccggcgcgttagattgtgctctgaagactgtgcgcgcggagggtcccatggcgctttataaggggtttattcctacgatctcgaggcagggaccgttcactgtggtgctgttcgtgacactggaacaggttcgcaagttgcttaaggatttctgatctagagcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaaggcgcgccagtcccgggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtatttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtcgagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtatgacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 19 - DNA, 1500 bp, Zea mays
agttttcgcttgtctattcaccctctataggcaactttcaattatgtaatcactttttttttcttttttctgtttaaaatctcagtttcaaacttccaattgattttgaatacgaggtttgggtttaaattcatattggaggcaaaaatcgaaagttccacgtgatgctaggttttatttcggttttctatctcctattgtttttcacgtttcaacttgattcaaattctagttttttttaacttaagcacaattaaatacaacataaaaacaacatggattcaagttctatttcaatttttattaactattatgttgtctagtctgttcaagcacataatacttataaatataaaattaaacgaaatcacatatttccacaaatcttgggtactacactcggagacgacgatggattccatctcaatttggatgttgattatagctctatttcagttgtcactgttgtcctaacacgccctattgtgcatgatagtgcacgtgctcaacgtaaaagaaaagagatcagtaacaagtagcagcactgtacaaggtaagccgtgattcaattaaaactgtttgagcaattcagttgctagatcgttccaccatcgataattcgatatgtacgatgatataaaaagagcccataagtttgtcttgaaaaggttgatcaaataatttaaattagatgataaaaaacatggaagatgtgggagtggacgacggctatgaagaatagtactatatcaggtttatacgtaaaatttatttttgaaatgtttttataatctgtttgaattgtattttttgcttaattatgtgattggatgttttttcatgaaatgtcgagttttattttaaataaaattctgtaaagagaagttgctgcgctgagaaaactataaatcgatagtaaaggctgtacgcaacgtttaagtccttgtttgaatgcgtatgaatctgagaaagttcagaatgattaaatcttttttatttaattttaatttgagagagattaagttctctccaattctctttaatttagacgtaatcgaacaagctggttgccaaactagatgagtacattttgtccactgccatagagccatcgactacaaaagtctagaacacagtggaaagcaccagacaacgcgcgaccaaaagggcccaggccccagcgccccagtccgggggttgtgttcgccgacctgtgcgtgcctgctcgtcacgtcacgtccctatttgcccgtcttcctcccctccagacccttctcgaacgccccttcgttctggatccaacggtcggtctctgccgggctcgaacgttctcgaaaccacgtcacccccgataaaaccccacgcacagcctcctcccttcctcaaccatcattgcaaaagcgaagcaagcaatccgaattctctgcgatttctctagatctcgaccacccctactagttttggttcctcctttcgttcgagagagcgtttctagtggca

SEQ ID NO: 20 - DNA, 1500 bp, Zea mays
caacttacaagcgatgaggccaagacgattagacgaatagctacagaacaagacaatgagagttcagcactcactttttgccagttccttctccttggcagcagccaggcgcttgagtttagcagcttgtgcaaatgtggacggcctacagcagacatacaggcaaagaagcgaggagtaatttgcagttggaaatcattcttcgatcaatagggaaactctgagtcacagcgaaaggaaggttaattgcctacgttgacaactgatcagcctccttgagaagttgcttgatttcaagccgcactttgatctgctcatcactaagtcctccgctctggatgacaaaagcacagaacgcatgagtggcaagtggaaacactagagcgaaataaatacaaaaccgcagactacaggctaacagatagggagaccgggaagacaaagactcgagcctgcattcaacagttacagtcgcctcggccaaaggttgagaaatttgcatcaaaatccaaactgtctagggccatgggaaatagttcctcggaatcagagttcaattcatggacgaaatagatggaactgatggtaggctactcttccgcccaatcagaattcacggaagatccaggtctcgagactaggagacggatgggaggcgcaacgcgcgatggggaggggggcggcgctgacctttctggcgaggtcgaggtagcggtagagcagctgcagcgcggacacgatgaggaagacgaagatagccgccagggacatggtcgccggcggcggcggagcgaggctgagccggtctctccggcctccgatcggcgttaagttggggatcgtaacgtgacgtgtctcctctccacagatcgacacaaccggcctactcgggtgcacgacgccgcgacaagggtgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 21 - DNA, 1500 bp, Zea mays
tctcataaaagcaataaaacaatatctcacaaaatacaagtggcaaacattatacaaacatacacatagtcagaaagtcacaactcaggaccttaaaaaatgaaactatccgattgaaaatacattgataacaattgaacactagaaaataatatcacaaatcaaactatggagcatataactagccatataactcttataatacaataataaaatcatcatatatttaaataaaacactagcaagtctaataacatatgactatagaatcaagatgtgtatgatgacatgacacttgcaattttatcatctcctactactcgacatagtcaatataattgatgtcctccttatctttaaagtttccatgcgaattataaatatatgtatgaagagtaatgattgataagaaactataaataagagtcacaatagttcaaacaactctaaactatatatcattagatagatcttgattttagaaaaataacgaaatcagtttcataattttctaagttaagatgaatttacaaagattagtttagatttaatattttttctgaaaaaataccgatttcggaaacgggcaaaagagatccaaactatttctgtttttttttaccgatttcatttccgtattttcggtaacggtttccggtttcgtatgaccctaaattttggtaaagtttcgaaaaaaaatattttaagaactgaaaattaacgttcctgttttcatccatactaatggctctttaccgctaaaatgttgcccacaatcattgagtaggtttagacgtgagagcaaacagtacaacattacgattcgcccttgcccaaatttacatgccttttccctacggaaacaacatagaatcaagttgacggggttacttacattgaagtggccaaactgatggtagctgtagatttggatgtatgttttctataaattagtcaaaattgagacaaaataaactgcaatttaaaactgaggaaatagtaaaaaaaaggtgaagaagggaggaagaggaaatcagaagcaaaaaatgggcaactttaggcccattatctcgatggtctcgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcgac

SEQ ID NO: 22 - DNA, 1500 bp, Zea mays
cgagaatatatgttatcttcgtcgttagagaaatctagacagtatacaacaagatccacgtactacaggtaaacttttaggggtattgtgaacaagaggatgagtaaactctaaaagaacaaagctccaatgaaaatttaggtttttatgtggttagtcatagggcaagttgcaaacaggtgttgatctaaaaaggaagtagtagggaaatgtgaagtgtctttgcgaggaattggaaaatgaagatcacattttctttgggtgcatcatgggaagaaccatttgggactcttttaaggaggcctaagaatgccataaagtttgcaagatctttttgaagagtgtctacctataaacaatagtaaatatcatgtcaaaattttcatcttcgccattattctttaggagaatttagaatgttccgaataaaatatggatagaaaagaagttcccaaagtcatccaattttctacaaaatcttcaactttaagattgagagtgggtgttgtaaagttcttggaagatgagttgaaccccatggaggcgttggctaaagtactgaaagcaatctaaagacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagaggccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggattcacccgttcgcctctcaccttttcgctgtactcactcgccacacacaccccctctccagctccgttggagctccggacagcagcaggcgcggggcggtcacgtagtaagcagctctcggctccctctccccttgctccat

SEQ ID NO: 23 - DNA, 1500 bp, Zea mays
cgataagaacaatgttggacacaacttaagtctgttttacaacaatgtctctcaaaactatagttttacaatattatactttgcaattatcatgacaataatgtagtttcggtagctccaaaaatacagtagttttgagaaacattgtttagatacaatattataaatcatgtattagacaaaagatagccatgccattaaaactttgaattggactgtagttttttcaatactccaaaaatattatggtacctagaatacgatgtctagaaaacatattttttaaaatgcaaccaaacatcatatgacataaataatatagtatttttttgaaaaccatggtattacctaaaaactacagaatacttcattctgaaataggtcctaacaagttgcagcagctaggtcgtacatcagcaaatagctacttcatcaatctcagaataaacatattttatagatgagttaaactaaaaatatagaagaacaacgtacacgcgttgaatcacaacgtagcgcgatatccattcaactttttggaagtttttactgagcacaaattcgaaaatgggaagcgccacgtaacacgagcgctgggccaatttctgccagtgccagttatcccggcccacatccaatcctggggaagacgcgaacccggctccgcggcacgagttgtccgcacgtacggcacgtcggggctggctcgtccgcccgcgagtgggaggccactgtttcctctgcctcaccgggtcgtgtggcggaggggcgtggggccatggttcgcagcgcggggcgacgagcgcgctcctcctctcgcgcagcgccagcgccaccccgcaccgtggctttatatacacccctcctcccaaccctaccgaatcatcactaccaccgctctctcttcctctcctccatctctcaacgcctgaagctcaccgcacctcccctcctcgccgcggatcccccactactccggtaaccgtctctccattcaccctgcctgctgtctcgctagaatcgcctgcctctgccagcgccgtgacgcgggggcgcggtatggctctcccagatccgcctggcattgctcgctcgggtcgtgccaggccgatctgatctcgcatttgctgcgcgctcctcctgctgcggatcccaccggatctcgctggaatcggagcgcgcgtctctttgaaatgccgcagatctgcgtgcttgcgcgcgtgatctaagtccgggcctttcgttaacgaaatggtccgatctgtggtttggtggaggcaatgccatggtttttccccgtgaattttttttgctgattttaggagcttttttctactgtcctatgttagtaggacaaaaaaaaagaaacatagattagcttcaataggcgccttttagaacagattctgtacagcaactcgtggaaacaaatctgcttccttaatgatgttgcttgttttaacaaatgcggcatcgggcgagcttttctgtaggtagaaa

SEQ ID NO: 24 - DNA, 1694 bp, Zea mays
cacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtatggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcatacggtatttatttaagcacctgttgctgctatagggcacttgtattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgacgataagtcaagatcagatgcacttgttttaaatattgttgtctgaagaaataagtactgacagttttttgatgcattgatctgcttgtttgttgtaacaaaattttaaaataaagagttccctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcgg

SEQ ID NO: 25 - DNA, 1500 bp, Zea mays
tttaaatttggaacgtcgatccaacatctaacagaagcaccaattttacaaagaacccctttcaccttcctcacttggtgggacggttcttaatcaaattaactgcagccgctggtatacatgtacatgtgggcccgcctagcccggcacggcacaggcccacaaaaacacggtccacaaaagcacgacccacaaaagcacatatctaattatgggccgtgccgtgccagcacgtgtgcccagtcatcggcccacaattagttatgtgtgccaggccgacccaaatagcccaaaataccttaatatgccagaccggctcatatacatacaacagtaatacatcaacaaaacgtataaaatatatatatgaccaaaataaaactaagatgttttgtggatgcacattataaacctttggtcagaaagaaaaaaatattacaactagctcacaaaaaatatccagttctctgtttagtgtttaattgagtactatacatccatacagaataaatatacaatgatcatcatcactattcactatccatatctaggtattggttctcgatggcttattaaagctctagattctccaagttatgctagtcatgtgggctttgacagaccttagttaaatactgagtctatattttgtgggccttagttaaatgggtcgtggcaggccggcccgtgggcttgacttgaggcccaggcacggcccacaatgtgggccgtgccggcccatgcccacaattaggttgggcagtgccagatatgggccgtgccagaaattgtgtgctttgggccggcctattaggcacaacataaatgtacacctatagccgcatagccgctggatgtgagatgaatgtctcagatttaaaatgtgcacttgagcaccgtacctctttgaacaacagatatgttcctttaagattgatggtggaaaaaaattagtcagtacctcactgtatggcggcattgtttgattatttcagttcgcacccgttggaccttgctcattaaaaaagtttataccatggagtctttgcatgtagttgtgtagtaggggaagagtggcataggaggaatcacaacttcagctagcttctctagccttagggtatttttgtctttttgcagttcggtcttttcgcagccctgcgctgccccccctgtccgcctgtccctagacctgttttgcgtcggcggggaagacagttgacaggaaggacacgatcttcgtgtccgatgccgatcttcatgcgagcagcgagccactacgttgcgctgccagtgtcggctatggtatccaggcattcgttgtgcacgttgacgatgagctcgaagccggtccgggtgaacgcgagcagcacggtgaggtcaacgtcgtacatccgcacgtcgatgctgaggccagccagcagcggcatgacagattgcggcgtcaggagattgtgccagtaggtggcggggctgggggcagaccggcaggcgaggcct

SEQ ID NO: 26 - DNA, 1500 bp, Zea mays
caaaattttctattttttaaaaaatatgaattctagatttgggattgaacacatctaggctacaacgttgaattgatgaacaatagtgcttgttaataaattgctcacattcacattgtcgctcttacttcaaccatcatacatccatctacagtggtcacccatatttaatcctatggactaaagatgacagatgaacttctctcgttatatatatcactgtcctacatatatgagaaatgatatgtcctaaactcacctaaaaacaacaacatagtttaaatttaatcatagatgagcctacagaggtcgaacgtgatttggaaacatagctctattgttctctatctcatgcataaatatggtgcaatgaagaatattagggttatgatgtcgaaatctcactcgaactcgtgcctcatcataaatagcacactatcaattgttctatggctgttcaaatagggacaatcttgaaacaacatttctcacatgtaaaacgttgtgaagtatgccaactgaaacggatgacacatacacttcgtgaaccaatcgatattttacttgcttctatgttaaataatgttataatacaatattttattcaaatgctaaaacttattactagataaaaataaaatttaattatcttcaaaaactaaccaatagatattccatcataactacatttaccaaactaatatactaaaaaatataggataattactaaattaatcgtgcaataatcagtatttatgagattgataattttaaattttgtgggctacaaacaaaaattaaaacttacttttcaagttggagataagaacaatggtagacgtagctcgggatggtatggcgtcggtgcagacggttaccctttgtgcgaagtggcgcgggcacgagggtggggacttggtacatgcatgagagagaggaagaacgaaacaacttctcaaattaaagcatatgaaaatcacctaatttttgtctgtcggtggaaactaataactagtttttattatcttttttaataaggatccacgaaaattatttttgaccgatgaaaatcctggatcttcgtattatgtttcgccttttcccgactctttgcatgctagatttccatgcttggactaaaacgaagataataaaaccaatctatcattttcacacgatgtattcatacttgcaatagataaaccactactccgacgggatttgctttctgacctctgaaatcttggaaggattatgtgtctacacttctcgatcgaggggaaaaagtcgtagtaccaagttgtagttaaatttgtttcttcgatgacaaaacaaaggagaggggcccgcgcggcgcagcgcagcgcagttggctggttccggaacacgaaaaccaagcacactccaccagctgccatccaccgggttggatggagattacaatactcgaatagtcagccagccagccggcttgaacgtgcagttttcccctataaaacg

SEQ ID NO: 27 - DNA, 1500 bp, Zea mays
acacttgctctcttcgcgtggtcatttagcccccgaacattccaagaaaaaatagcacatttttgattcataaggtaaagactgccactccacttaacacagcacgctgccaccacacatggattagcaggagagcctgctgtaaaatcctaacaggagggagaacctccaaacaagggttcgccgagcaaaaacacagcccgaccacaaccgacaacctgaaagaacaacagagatacacaggcatgctgggggacctagaccagcgcccagaagtaataacgccagcggagatacaaccgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctggtcatcagtagtagtggccttttgcccttgcccctctctctcacctctcttttctcagccttgcttccgatggatcccatcccactgccatcctttctttcccttgcgcgcattgcctagccggccggccggcctgctattaaaccactttacccgccccctctcgctcacgctcgacgcagctcccttttccttgtttgcttattgcaagtctctgcaagaacctgctagagaggaacaaggtagagtagtatcgcttttttccatctaggttatctctttttacatgaaaaatttcagccgtatttcgttctccatcagtcctgcgataatatatacgcgcgtcttgtgtgatccggcatatgtatagttcctgctaactgatcgagatcgctctcgtttgtactttctccctttgaggaaagagtttccccttttctgtgcttcaagttcttgtaaggaaaaccatgcctgccagcttcttctgctacttgtatgatgattcttatttgcttattacttgatttccgttttttttcttgctttctatatgtatgtatctgggctgtcttcccctgcgtctcgttactgctaagctttggaaggtttcaactctttgtatacgatgaggtttctgctcctagtagcagatccgcgcatatgactagatgtttgaggaaaagaaaagggcaagacgctatatatatatgcagcacgcagtcgcacatatattcagttttccaatct

SEQ ID NO: 28 - DNA, 1500 bp, Oryza sativa
gcagctgttttcgcggtacagggtgcaacaaaagcccatgacggcccacacctgcctctctccgctccaaacaccgaaacaagggggtgggtgcaatgggccggcgctcgaagaccgcgaactctttccaacagcccagcgcattagcccctcctcctactctctctaccttctttttaacatgcgactttctttctgtggacgacggcatcaacgacgggagcaggagcgggggctgaagcacggtgcgtgggctcctggagtggcgacggcctctccggcgagcttcctctggcgaactccctccgctcctcctatggcgaaatccaaacaagggtcagtttcgactccaaccttctcccaccaccacctcctgaccgtgccaccacccggccttgtcggcactgaaaggcgtcaacttgtcagcgcgggcctgctcggtcggtctcctcctcccctatttcgtttagctttgcccccgccaccaacaccggcccacggcccatggccgaccccgcggctttggcgccgccatcgctatctcgccgctgtccttttttcatgaccttcggtgccatccctctaaattcgatgcacctccctggctctatctccctttacctccgaaatcctaaccctacccataatctctagtgagtcttgtctttatttatggcctctttgaatcgcaggattgataaaacgtaggattttgataggaatgtaagtgtaaaacacatgattgtaaaatagaggaaaaacataggaatggccgtttgattgaaccgcagaaaaaacacaggaattagatgagagagatagactcaaagttactaagagattgaagcttttgctaaatttcctccaaaatctctataggattggccattccatagaaatttcaaaagatttaataggattcaatcctttgtttcaaaaaacttcatagaaaatttttctatagaattaaaatcctctaaaattcctatgttttttctccaattcaaaggggcccttaggttggaatttggaaagtgttcgcgagaaatcaagcggtcgcacgttagcgaattaggatttccggaaacaaaggaccgactccgcctatccatcgtcacgagcacagtgtagaacctcccagacctcaagagaccgttcaaaaagcgcgcgcccaagcggggcccaccaacgcgtccccaccgtgtcgcctcctgattggttgtcccctcttcctttcacgcgaaccggcaccctcccgacccttccagaacccccaatccgacggccaggatcgcccgcgcgcgaacgttctagacccccgccacctccgccacaaaacctctgcccctcccctctccccccgcttcgtctcgttcgagaaatcagaaagagagagaaattcccacgcagcagcaagcaatccaatccgagagcgcgcgtttgcgattattcgctttcgattccgcgaggtttttggagagggaggagaaggaggaggag

SEQ ID NO: 29 - DNA, 1500 bp, Oryza sativa
acagcatttattgtagtctggtcaagcgtgtcacgctgcatgcaacgcagtacagcgcgttcctttacccggtctgtgaccagtcacagaccggtcagatcacgggttaggtggcgactggcggtctgacgcacgccttgccccatcccgtcaagacgaaagcctctaggcactcgtctcaagccggagctagcgtgttatctcttagagatggcacgttagccctggttagatttataccaggcttcatcctaaccattacaggcaaggtgttacacgaagaagggcaaaacatgcacgttgttaaactgacgcgtgggggacaagaatgaccggtctgacactggtcgcatcagcaacgggcagccacgatcccgcgtcatctccgtctccgccgggagtggaggtaggtgtgggctgtcccatcagaagggctcccggatggaaaccgtaccgatctccgcccattaaagagaaaaagaacagtccagtttggaaagagaagggtgcatgtggtatccccttgaagtataaaaggaggaccttgcccatagagaagggggttgattctttccagattcagagcctagaacgagggagaggtgggctcacactttgtaacttgtccatacacaaatccacaaaaacacaggagtagggtattacgcttccgagcggcccgaacctgtatagatcgtccgtgtctcgcgtttcttgctggctgacgatccttccacatacagagagagagagagcttgggatctcaccctaagcccccggccgaaccggcaaaggggggcctgcgcggtctcccggtgaggagcctcgagctccgtcagacatgttcagtttcattatattatgaaatgtcacgtactgtttgttctagttagtgaattgtcatatggtaagaatatataaaaattaggttttctggactctatcttccaatgtatttttggatcctataacaaaatattttcataaatatattttttaagaatctaaacttttttgaaataaaagagcaacaaagaaaataaaaacgctctctcgtaagtaactcgtgaagatccatcgagagccactcgtttgaatcgtcgacacaaaagaacacttcattgattgcttttcgtcaattagccgcacagcacagtactctccaatctgctaaaccaaaaccaatctcatccatccatacccttcttgacaccaagtggcaactcctgattggacgcgccctatcctacatggcacccccaagattctctcgataggctacaggggccacaccgaccctccacgtcatcgtccacgtcaccctcatcccggcccatccagccaatcccagcccagcaaaaaatcttcccaagtggccaccagataagcctctccacgtattaatacgccaagtgttcgtcgccatgacacagcacgcacacacaccccaccagcagcagcagcagtagctgagcttgaagcagcagagcgaggtagac

SEQ ID NO: 30 - DNA, 1500 bp, Oryza sativa
tccacctctgttggttgcatcgacgtcgcttccctagctcccgtctctagtccggatcctattcctccttggagaccgaagctaccgcaaccattgctcggtggttagcgagcgtggagctgtcctccccactttcgcgtcctcgttcgccaccacagccatacttcgcatggtgatgtcttctccttcactcaccgctaaactcagtgcaaccgtttctaccctagccccggccgccgctctcatagaggtgaaagttcatttacatgtaggtcccacatgttttatgttttttatttttcttttactgattagcatgccacgtaaatcaaaacaacaatccatagtgttttaagtatttttatttaatacgtgagatggagtacaaaaacgagagatgcaaagtgaacttgctaaaacacattttctggttgattacagtcgcttgttgagccattggatcggtcataggattcgtgctagcatacttaattacgcgtaactagttgtgctttataggttacaggtcgctaattagcggtctactggagaactttgctactatttttttcttcactgcatgcactcgatcaagtatgagtatttgtaccgaccagcgaaacacatatgtaattaaagtataaatatgtaattagtatatattagtagtatatttagacagtagttacaccctacatacacaccacttacatatataattagtatgtaattttgtaacttacatatgtaattttagtacttacatatgtaattttgagacttacattgtaaatacactaaaattacatatgtaatttagtaacctacaatgtaaatacatgccgactaacttttgatgaaaaatatggtgttataaatatagctactcccgaactttattccttctctgtgagatatcagtggaaacgctcggtggaatcgggggagtatttgggagcacgcgccgacgcgcgcgtcgtgcgtgccgtcgtctttgtcgcggtggagcggagcgcgcccacttgcgcgcctgggccggaggcgggcgcgccgggggttcgggaatcccctggagccacacgtaaaggcgcgggcgggagggagggaggggccagctaggataaggcacgcgcggccgctgcgattggggcgcttgtgaacaccggggcgccacgtggagaggacgttacactccagccgccaaatttccactcccacacccgcgctcccctcccctctcttttccgtgatcgcacctcgcccacgcgccccccgccacacacaatctctgcagctctccagcttcgttggaactcgcgaatctctctccgatcccaggtaaagcagcgaacgacgtcacgcacgacgctgctcggtggatttcgttccttgctggggaaaaccatgcagagacgaaggtgaatgatctgcttttgtgtacttgcgtttaccaggtgaagcgcgagcttggagttggaggggagatcgatcagggccagg

SEQ ID NO: 31 - DNA, 1500 bp, Oryza sativa
ataattaattaattaatcaatcacttttcgtgctgtaaaaaatctcacccgatttgctgaaacgaactgagccgggcgactgtgatattctttcacgatttctgtttgtggcagtgggacattgctgtttattcgaaacaattttcaagtaaaaaaaaatactcaatggtaaggttgctagtaatagtttaacagtttgtttgcagctcagcaaatttcgtttcctcacagatgacacataactgaaagcactcaatgtaatgttgtgcttagctgctaaagcatgtcacgtcttagaaaacaactactccaccatggagaatttttcctcctacttactcctcacatacttaccatctccatataagttcccttgtcgtatcatatgtcttattcttcttgagcacagttattacagcagattttgtagaatagttatcgcatcaaaattttcctatgtcacctttgatcatgtgttatgtgtgcctcttgagtcttagggttaatgtggttgtaatgtgtttaaaaaactatatgaaagctcgtgtgttgctacgggagagagatacctcgaatgaatgtgagagatctccatttgagttgtgtaccttgagagagtgaaagatcacactatttatagacggttaataatggttactgaggtcgattcaccacatcgtcttaaacatttaatgagcatcctccacgtgaaaagtagagatgatagcgtgtaagagtggttcggccgatatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcataag

SEQ ID NO: 32 - DNA, 1945 bp, Oryza sativa
aaggtttcatgcgtatcgtgacagatgttacataatgacaaattccccagctggagcacctttatccctgctgtttgcatgaaattagcttgtcttgtagttccctccagcaaaaagaagtctgaaacaaaacaacatttcgaaaaaaaggcatccatgagttagcatttctacagttgtctatagaggggaaggctgcacgacaaagtttccaggcttggaaacaacctcttatgtaaaatttttcgtatgtatcagatgatttgtttgcgttacggcatctccacctaacatcaccttcatcatgcgcctatggtctttctcttgcctgttttatacgtaaaattggaaacgacagaaacttttgccatctttattaaaggaaggcaaatatgcaaatataggcatcaagatcacagttagtggattatcatctttgtaggttaacatgtcctaccccaggggagcttatactcaagtactccatgcattttcatgaaatgagaaaaaacgatttttaagagaaatgtactttcttgtatttatgccaaatggcaaggactgaaagggaaaaactaagaaagggaacgttacagtaaggctctgtggggactggggacttcagagaaacgtgaaccctgcttccttcctctgcatgaacataacaccagaggtttccagcctttcacacagttgttgatggcttcacacaattcatctctacctcctgactctttataaggacccccagcatcaccacaattgcacaagtacaggcattagatccacaagaacacttgggcaggcaagcacctctttgatctttaagccgttgttatgttctatttctgagcatatggtttctagttatattctttttcttcattcgtttcatatctttgaagtgttgatgcaaatgcggtgaacaactatcaactgtgtactctccaagtgaatgcgaataatcatttcctgtgagaattgtgggctagataaacgaatgaaatgctgttttatctatgtcatgtgtggaaatttagttaattttccggtctttttatgcattgagatgggtatgctgtttttttagttgggtcccatcatcttgagaattctttcaaatttccttttctttatcctatataaaggatagagaaggcgtatgcctaggtgcaccaaccctgaaagttttattctaattgcgggaatggtttgtaatttttgcttgttcaggttctttttcgtggcctttcttttttttccccttattttgcttagtctttcacagtccaatttttgggaagtagtatatcttagtttggtcctaaggcaccatgttgtactgcaggaaaaaaaagagtaattgtattctgttttttccttgattactatatccctgttttaattaattttgtgcctttgttgtttgatgttggaacttcaatgcccataattagtcatttgacttgttttgggttttgacgctatcttgagtgccataggaaactggtagaatttagtaataattttatatagactgaatgttgagcccaccacaaatggtttccttctgtacaagtatttaataactcaagcacaggaaacatcagatctctaatctaaaggttaacaatgggctcaagcaggagcagtagttcagctctatctgtatatttagaagggctggatctacctgtccaccagcttttaattttaccctggcagctggataacttcttgtctgttaatttcatttagtgctgtgttattttcttcttgttgttcaggatggatgcttttgaatttctggaatttcgtattttgttctatctctttatgaaatgacgttatggcacactttttctgcatattcttgatgaaaataattacctagtcatttttttagttgcaggtttgtctgggactttgagtacccatgcaattc

SEQ ID NO: 33 - DNA, 2315 bp, Oryza sativa
gttcaagatttatttttggtatttaatttacttgcttaagtcagatatattcccatcgttgcaggtttgtcacttagtattattattaagcgctctagcactaggactctggataaataagaaagtttattcacgaggctagagtagtaatcaataacataagcgtggtgtctaggtcagcggttatcttcatatgtagtgtgctccatggaaagtgaggtaggaggaaggtggtgacagtcccgtccgtcctttgtatccctccatgttcgggtatatcatagagctacaggctagacttagcttggcagactaggggagagccggtgctcgaagcaatccatgaggctttacatttaacataagttagtaaattaacccataggaatcatctctagactgaacctaccagtagttgtgcttggatataattatattcctacatatacatacacgttccctgcgattagatacccttggaatactctaaggtgaagtgctacagcggtatccgtgcgcttgcggatttatctgtgaccgtatcaaataccaacaggtagatacaaggaatcatctctcctatccattggtttatcatcttttaaaattatctcttgctctcctattgcctctgcaactgcggataggtgtttctcaacaatgaaggttgtgaagaatgctttgtgcaacaagatggatgacaagtatctcagccatagcctcatttgctttgtagaaaaggatatgtcggacacaatcactaagtatcaccgtggaaaggatgcactgtatgccctatctatatttaccatttagtaatatttatatggcttgtgctaactttatgttgtctttacaggcaataacattatttggaaggcatatctatatattactatttaagataatgtaatatctcaaagtttttataagctgcaatgaggtgagtttcacttagctttctaacttgttatgagttatagatgcatgccaccagtcattttttatcttgcatcagcccctgcctgttagaatatgtttctttgtctgggagtccatgtcaactagccaatttccaaatatatgaacaaaactatgtggcctttgtaacccaaatgagataaagactactctccatagaaatttagcaaacatggcactcaaagaaaatgtgttggatagtttcatcatgcatacaaaagcaacacttttgaactaccattccaaatcctttttgtaaattatctttgcttaacactacccctttgagcaaatgtggctttgtgcggaaaaaactcaaacttggtagggtagacatccatttatataattggatccatgtacataagttgttgagtacttcaagtacttacccttgtgatatacatctcaaatatattgaagaagagaagttctttttttgagagaggttgaagaagagaagtttgtccatagctgaagaggagttttatagtgtctagcttaccttgctgctgattgcatgtctaaaatgtcgtttaatttgggctataatgaaatattcaccaatatttctgctggtctattaaagtttaatagttactcgtaactcatttattttgggctataatttaatattcacctatgtttttgttagtctattttatttccctagtgtgcactagcttaaccccaaattagttttgaacacttaacctaaatgtgtctattatggtcagacactctctcacggcactctaacaaaaagtgaattttgttgttatgtttttgtcatgatctcacaagcaatgtacatgtacgtttctagagtgcaatcttatgctagcctgattgtgaatttagtgtagtttgttttctctttttgtagctacactaccaataacctattgtcctctagtcataccacgtaatcacaaggcaaatccctaactctcacctttaaaagcatgtctttattttcttgggtggcactaatacaaaatctttttcagcattcctatgtgcgatagcaagaaaacatggcataactcttgcttcactctaacaaaaaaaacacttttccaactttaaaacaatggtatctatgtgtttaatgatcaatcaagcatataatgacttacaagtttttacctatgccctttttgcatcatcttgtttgcaacagacaaactagatattcctttaggctataaacacatcagcatgataaagagattaggtaagtttgttatccctttttgcatatattctcgtctactccgtgtatataagcccctctcctccaactcgtccatccatcaccaagagcagtggga

SEQ ID NO: 34 - DNA, 1194 bp, Oryza sativa
ttgcatgccgtcgtcttaagcgtccgcgtgtgaaaatcggattttcgcatacggttgaaccggtcgcatgcaaagatcgcgatcttcgcagacgatttggcacatgcggttgcaccaaccgtatgcgaaaacccttctcgcccgtatgcaaaaaccatctttgttgtagtgtacggttcacaatggtttggatgggaaatcattgtgaaccaaaagtgatagactgatttcgacgagtgtttttttttaagtagtgccacaattttggtcatcatacgtcgtgtctaaaattgtaacttttgaaaaccaatttacattaaattaaatttataagactaaataaagacgatggtcattgaacaattgttgagaaaaatctacacacatgtgtgtccaacacaaatgtttacacatatactactatgttcatagtcgaagttagattttttttttccttaaagggaaagtctgttttcaaattttagacctcactccttccgtttcaaatatatcgtgtatttttttttctagggcaagcttttgaccaatgattactctattatgacacaatgttaaagggatagattcatattcaaaattactattataattataattttgtcatataaataatattttaagcaattgttagccaaaatctcgtcctaacgaaacaaaatacgccttatttttaaaaacacggagtatatccttaaatatttctctatccaatataaaaggtcaatcttttaaaattccgatcatcaataatttctcaaataattactttgaaataaaaaaacatatgcaaatttgtgtcgtcataatatccaatgaacttattcaaatttataaacttattttaattcaaaatttgatcattaattttttttttaaaaaaaaaccaaatcttatcataaacgtcaaatatatttttgatagtgggggcgataataccataaaactaacaacagaagagacatgatactactactgtaatcctaatacgtacgtacgtatacttctacgccggatgcataacttcagccttgtgagacacaacagttgctgcctagctcgtggtcgttggttttttcgctcgagaaaccactacgcgtaaaccgtgaagtatattatatatagccaactggtcttctcgcaaatccgcacatccctttctgcccctcgtcttct

SEQ ID NO: 35 - DNA, 1500 bp, Oryza sativa
gcaaagaaggccagtggcctttgcagctaagctagctagctagcccttcttcctctctttcctgctttccctttgccttctcctattaatcctctgcacctcacacagcagcagaaaacccaccaactggagctctcctttcctactccaagaaacgaaggtagagaaagaaagatcagatcagcttcaggaccaattttagctaggttatatatctctttgcgtgctaatgtgttttagttatctgggtgtgtgtagagttctttgttaaggcactgattcagctgcagtttagattcaagtttgtatgttctctctttgaggaaaagaaacccttttcctgtgcttcgagttcttgcaaagagaaactgtgatgcttggcttccagtttgatgcttctttgttcagattggaaattcttcctagcttctttctctatttatgtagcaaggattctttccggcccagtgatcctggtttcttttggaaggtttcagttttttcgttctttcttgaaatttctcttcttgccttaggcagatctttgatcttgtgaggagacaggagaaaaggaagaagctagtttcctgcggccgacctcttgcttctcactttgtgatgagttttctttggtcaattcttagctagatatgttaagatagttagttaagcaaatcgaaattgctagcttttccatgctttcttaaacatgattcttcagatttggttggttcttttttttcctttttgtggagacgtgctgttcttgcatcttatccttcttgattcatctacccatctggttctttgagctttctttttcgcttcttcccttcattatttcgagcaatctctgcacatctgaaagttttgtttcttgagactacttttgctagatcttgtttactcgatcactctatacttgcatctaggctcctttctaaataggcgatgattgagctttgcttatgtcaaatgatgggatagatattgtcccagtctccaaatttgatccatatccgccaagtctttcatcatctttttctttcttttttatgagcaaaaatcatctttttctttcaaagttcagcttttttctcttgttttacccctctttagctatagctggtttcttattccttttggatttacatgtataaaacatgcttgaatttgttagatcgatcactttatacacatactatgtgaatcacgatctcagatctctcagtatagttgaattcattaatttcttagatcgatcagcgtgtgatgtagtactgtaaatcactactagatctttcatcagtctcttttctgcatctatcaatttctcatgcaagttttagttgtttctttaatccggtctctctctcttttttaatcagctgagagtttgtgctgttctttaatcattaccagatctttcatcagtactctctcttctgcatctatcaaacttctcatgcaatgtttttgctgttctttgatctgatctct

SEQ ID NO: 36 - DNA, 1148 bp, Glycine max
gcaacagaagacccaaaactcaaaaaagttagtttcgggccaacatttcctcttgagggatgacacgtgacctgctactctggcccttatctggcatgtccatccttcttggcgcgacatttaattcgtcgtcagaaataactgaaggacaccttgcttgtttctcttttggccgccaccggtcttgtcatcgtcgaaggcgcccttgcgcttgtcggcagaacctttttcggcgacctccttgccttttcctttggccttgttcgtcatttctacagagaatgcaatgagaccaacgccaattgcatggttagagttagagaaatggagagaggaagaagtgcgtgactagagtgtgtgtaactgtgaagaacgacgagtccaaaatgaattttactgtaaataatttgaggaaaaaagtgatcaatacatatcatgcggtgcatacaagaatcggccattggtcaacttgtgagaggaaaaaatcatttaactaataccaaataatcttaaaattaataaaataatttaactaattaacccacggaagaaccttcttccgttgactctggcggaagaagttcttccgcatagttccatggaagatggttcttccgcagttcttctttcgttgacactcgcggaagaaatgttccacgggcgtccgcggaagaactttcttccgcaaagctaaagagcatttttgccatgtcgaaatcatcgccaatgaccagggtaacagaaccacgccctcttatgttggtttcaccgattcagagcgtttgatcggtgatgccgccaagaatcaggtcgccatgaaccccgtcaacaccgtcttcggtaagatccctagccgacacttcgccttttcaggatttgcattgttcctagatttttggatctgttgtttgaaactccacttttctattttggtaatttttagttttattttgtaatcctgctgtttatatgtcttattgttattattaatcgttgcatggtctgaactggtttagaactctacttgtattgtttgttaaaatcttatttgaaatcgaatagtaatataattttaatcgaatggtgatatgcataaacatcgtatttgttcgtcgaattctggttttgaattgaataatattgttatg

SEQ ID NO: 37 - DNA, 1378 bp, Glycine max
ctagaaattaaatgtttttaacaggtaatttgagaaaaatgtacttcaaaataattagttttaccagtttatgtcttctttttctcttttttatctttattctatgtttcaaattctaataatacatcatttaaatatttttaatttaaaagtgcttactaaattttaaaaaaatcatatttatcaaataacttctactttaaatttaaacttcattatttttaacttaaaaataacttttaaattaaaaaaatgaaaacaaacactacctaaaccctaaacactatctatctaagtcacattacttaatgattcttaatttatgttctttgtaaactttcatttcttcctccttttggctatacatgttcatttctgtgtactttactatattattagtaaaagccttttatataggtatatcaaatcaaataattaatataatatataattctcttaatttcatttcttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaag

SEQ ID NO: 38 - DNA, 1500 bp, Glycine max
gatcctcacaaacctcacttggagacataggtgtgagggtaacctttttccctttatgtacaaatgaaaatttgtttgtgacaccattatggacaacatccttacactactaaaaaagcttttttttacgacatcatatttacgacagtcatacaaaaacgtcttagtatgtataaggatggcaatttcgtaaatatttcaaacatttcaaaggcagtttcagaaaaccgtctttgaatgcggccattttaatttttaacgcgcccctcgcatccgttcctcttctttccgcaaatgtggtgctcgttccttttctttcccagctggcatctgttcctctccccactcgctagctatcttctgcttctcctcttctctcctcttcccattacatttctccaccttctccctggtaccaccaccgccccccactccacattcgtcctccgcccccattcccctatcctccagtaaaattacaaaaaaccctaacaccaaaaaaacccaaacccctgtcgcaatgaaatctccacccccaaatagctctttggaatagaatcaaggaacttaccaaatccattatatgctattggggttttggcatgtttccggtgtgaaagaaggaaaaagaaatgcgtatgcgatggtgatgtacgtaggtacgccgaaggactacgaattctacatagccatactcgtgcttctcaaatcgctggctacgctcgacgttgaaattgatcttgctgtgattgcttcccttgatgttcctcctcgatggattcgagctctgtaagtctcactccttcaccatcatttgccactttatttttatgtacttttactttattattatttgtaacctgtatttttatttggtttcggatatctgttgctttattattcaccctggaatttggttgattttattatttttgaaaaataaggaaagagatttatttgttagcttaattgttttaattggcgaatatgtttttcttttcccttttttgcacagagtgaagctttgttcttagggtaatggattcccttttttgtgatgctagtggatgatttgactgattagtgtttagtggaatgaagaaccagaactagtagtaggtagagggaatcacttttggttttggatgtaaacttagaaatgtgcagcactgcacagaattgatatttgatcgtgggtcaaattgtcaaaatgtgcaaagaatacaaaggcacaggtgatatcattccattttacgttttttaacgaagctgttagtttcaattcaattatttacatatataataaatatattgatacttgctttagtttcatgaattaaaagaatttgattttgtaaatttcatttgaatttgtttttgtacaagctctcaacttttattatatgaacgagaagtttcttttttcctttttgagtttatttgaacttgtggtgttctaattgtatatatttttgtgcaggtgtcaatcggtactactac

SEQ ID NO: 39 - DNA, 1261 bp, Glycine max
atctctcgacagttgcgaactgaacgctgagttggtaatgctatgccctatcgctttttgcaccgtcccatgatcatttcccccacaccaccccatcaacctctaaaaagttaagagtgaaaattacacacacccgaggagaagaaaagctgcttcttctaagcatcacaacctagttactttacttgtagggccttttccatttcccctaaattacccctcttttcatcatatgataataatatccagctcagactatagtatgatattatgatgtcagcataataggttggcactaaagtcttaaagggcattgtacatgttgcacctggcattcaaattcataaatactaacactgtgaaatagattataaatcctcaaataaatgtcacacggttggggttcgaatccactcaaaaaggctaatgggatgggatttaagtgccaaggaatataccatggactttaacagcaacacaatttacaatctaaaatgtattacttttttttttcaaaaaagatatacaaaataaggtaccaagaataaaaggagtatttagaaacagtggcaccaatttaataaattatttatataaaatgacacttatttaatttatcaatgataaaagtaatattgatttattctctgattaactgttcaattaatagtgttattatcataatctgtcgcaaaagttatttttatcaacaacaataattgatacaagtagtataaaattaagcctcttagttaatatagactacttgatactaaaaccatgttacaccaaaaagtaatttttatgtcacttgtctatataataattacgactaaattaataatttttaaaaatattactgaatccattaaccgaacttttataatgaaagtatttttatgctttaaaatcacaaacattgaataaactaaaaatgataccacggaattggaacaagagacgttccacacaaaagaaaaaaatatgttgaataattgaaacggtgacaagaaaagtggaataataatacaaagatggcagatggggttattgttattggaggagatgagtgaaataatgagtgaggggggtgtaactggaaagcaagaaaaagcgcaagagtgccagctatttccaacaacaaacgtggcccgtgggatgcgatattcgtaacgaacggcgaggatggaaggacgtgcaatttgcgcttcatttgaggcgaatttcatttggccagaccttccttttttaaaccacaggg

SEQ ID NO: 40 - DNA, 1094 bp, Glycine max
tgtgtcaatgttgtttctggtgaattgacataatgaattctacctgtacggagtagagaataactatttacccaacaagaatgattatctcattaatttttgaagtagacgcaataacgaatatattatacattcagaaaaatttcaccatattattctcaaatcacaacaataatttgttttttttttgcttgatataaaaccaatactctatactttttaaggttaatttaaacttaaagagtatttttaagatgcatgtactttaaggaataatagaaacatgacaacatcataaaagaatgaagaaactgaatcataacgtagtttgttacgccttccatttggtggttgatttggatacaatctagattggtttgctaaatggtttataagttatgtagacgtttttattactactattttagacaaatcaaatacacaccttcactttattctattcaaataacatgatttttcctaacattttttaaaaaaattactttttaaatataaactaattattttagaaatagttttataaaaatccacgccaaaaaaattaagttgtttttataaatataaacatcgggcttcaatcttaaatttataaatgtacgaaataatttgacagttaaatggaaattgctagcatggaagtgtttttatcatttatcaaactcaaccaaactgaacatcagaataattattagtgacaaattttgcagcatatgaagtggcttgcatagctccaaggctggcgatcatatgtcagattagagcaggctctctttggtactatgatacatttcaagcaaataacaaccgtaaaaattcacgccaaaatttttggaacgaatctatatattattattttatttcttttgatttcatgtacgtacagtgcccgtaattgacatgtctttgttccttaatgcctttcccacgtggaacaggcacctagaaacttggactaagtagggaattgagggccatggactatagtgccaaaccaacatcattttatatatatatatatatatatatatatatatgctattgttttctatagtttttggaaattaatacttatc

SEQ ID NO: 41 - DNA, 1449 bp, Glycine max
atttgtactaaaaaaaaatatgtagattaaattaaactccaattttaattggagaacaatacaaacaacacttaaaacctgtaattaatttttcttctttttaaaagtggttcaacaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttc

SEQ ID NO: 42 - DNA, 1321 bp, Glycine max
aaaaacacaaaaaaaaattatacaaaaatgtttctcacaacatgagaagtaaaatccctcaaagaatttcacatcatcatatcagaatcaaaggaatcaaaatcataggtcaaaaatacaaaaacaccaagaacactcaatttattaactaatttgcatcatgacatcaattggtccatcaaacacaacaatcttgtaattataatcgtaacgaaagaattacaatgcaataaacatcccaaaataaacctcaatttaatcctctaaggatccctatacatgttcattctaaccccaattgtgataaattcatcccttacctctaagcaggctcacgtgtgtagtctggcagtgatagaggcatctctagtggttttctaatagtcctcaagcttgtttttcctctagttgttctgttaggattttcaagcgttagagagaagaagaagagattggagcctctatttcactgttaccgtacaagggatatttttctcaccataaacattattttgcaaatcccaacgaaggagatgtccgtacataagttcgaaacctggtgctcgaatttcacgacgattcaatggttaacaagtccaagattgtatttttactgtgacagatttgagtgtatacaagaaaaagagagctccatgcgaggaatatttctctcacagtagacattatttcataaatcccaatggtaaaaatatgcaaaaatgagtttcaaacctgcttttaaaatttcatgacgactcaacggttaacgtgtccgggattatattttcactggaacaagtttgagtgcatgcgggaaaagagagggttttgggagaggaaaaaaggaaaacaaatttaagaggaagagagagcgtaaaaatttatcgtaaatgtaaaaaatgacctaatatatctctatttataactagggtactctcaatctattatttactcatttttttattttattattttataaaaaagaattttattttacttcctatcaaattaataaataaaacattcttcttattttctaagatcacatatttattttatttaccttaaaatcatcattttaattaataaaattatttcttcttatttatttaattacaaaaatcttattatttttttaaaattttatttatttttaaataaaatattttttaatttattttataaaaaatgagatgttacattgaattataaaataaatagccaacaataaatagccgacttgcttttgcattgactaaggaagtcaagtcatcaataaatataatttccagttggcaatattctcaaagttggtctatat

SEQ ID NO: 43 - DNA, 514 bp, Glycine max
agatttgatcgatacttcattaaattgacattttattttaacacataatacattattaaaaatataaataaacatttacagcgaagttatataattaaaagcctggtctatgtaatggtaggaaatttgaaaatctaaaagcaaacaaaaattgttgtttatggtgctaagttgcacctggaaagatgcattgtttagctaaaacattcacgtcgagtacttggtttgggaaaaaaagccattcaagcttagctggtcctctctcctgtctctctctctctgtctgtctctctctgtctgtctctctctcaagcacatacacaaacaaagtaagggctataaataggagggatggaagtggaagaaagtctatagcgaagtttcatttctttggattagaaatttttcccaaagctgatcgagaagccagccaggccaggtctgtagttttctttttttctttttaatattaattcattattgtgttcttcatcatataatataattaagcctt

SEQ ID NO: 44 - DNA, 702 bp, Glycine max
cgcgccgtacgtaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgc

SEQ ID NO: 45 - DNA, 579 bp, Glycine max
acgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgc

SEQ ID NO: 46 - protein, 563 aa, Camelina sativa
MESFALHSLSTTATSTLLSHHHPSRLSLLRRTSSRSPPSTISLRSHSVQPLSFPLLKPIPRFSTRIAAAPQDNAPPTPPPPPPSTPSLQLPQGAKLVPLILSVSVGLILRFAVSVPEGVTPQGWQLLSIFLSTIAGLVLSPLPVGAWAFIGLTASIVTKTLTFSAAFSAFTSEVIWLIVISFFFARGFVKTGLGDRIATYFVKWLGKSTLGLSYGLTLSEALIAPAMPSTTARAGGIFLPIIKSLSLSAGSKPGDPSSRKLGSYLIQSQFQCAGNSSALFLTAAAQNLLCLKLAEELGVVISNPWVSWFKAASLPAIISLLCTPYILYKLYPPETKDTPDAPGIAALKLKQMGPVTKNEWIMVGTMLLAVTLWICGETLGIPSVVAAMIGLSILLLLGVLNWDDCLSEKSAWDTLAWFAVLVGMAGQLTNLGVVTWMSDCVAKALQSLSLSWPAAFGLLQAAYFFIHYLFASQTGHVGALFSAFLAMNIAAGVPGIVAALALAYNTNLFGALTHYSSGQAAVYYGAGYVDLPDVFKIGFVMATINAIIWGVVGTFWWKFLGLY

SEQ ID NO: 47 - protein, 563 aa, Arabidopsis thaliana
MESFALHSLSTTATSTLLSHHHHHHPSRLSLLRRTSSRSPPSTISLRSLSVQPLSFPLLKPIPRFSTRIAAAPQDNAPPPPPPSPSPSPSPQGAKLIPLILSISVGLILRFAVPVPEGVTPQGWQLLSIFLSTIAGLVLSPLPVGAWAFIGLTASIVTKTLSFSAAFSAFTSEVIWLIVISFFFARGFVKTGLGDRIATYFVKWLGKSTLGLSYGLTLSEALIAPAMPSTTARAGGIFLPIIKSLSLSAGSKPNDSSSRKLGSYLIQSQFQCAGNSSALFLTAAAQNLLCLKLAEELGVVISNPWVSWFKAASLPAIISLLCTPLILYKLYPPETKDTPEAPGIAATKLKQMGPVTKNEWIMVGTMLLAVTLWICGETLGIPSVVAAMIGLSILLVLGVLNWDDCLSEKSAWDTLAWFAVLVGMAGQLTNLGVVTWMSDCVAKVLQSLSLSWPAAFGLLQAAYFFIHYLFASQTGHVGALFSAFLAMHIAAGVPGILAALALAYNTNLFGALTHYSSGQAAVYYGAGYVDLPDVFKIGFVMATINAIIWGVVGTFWWKFLGLY

SEQ ID NO: 48 - protein, 549 aa, Arabidopsis thaliana
MESLALRSISLSASYLSLHRSSSKSFALLPPSISVHTSPTLRSLSISSPRFTLRATASSLPEEQNKPQPPPPSPPQPQGAKLIPLAISVSIGLIVRFLIPRPEQVTSQGWQLLSIFLFTISGLVLGPLPVGAWAFIGLTASIVTKTLPFSTAFAAFTNELIWLIAISFFFARGFIKTGLGDRIATYFVKWLGKSTLGLSYGLAFCETLMGLIMPSTMARAGGVFLPVIKSLAISAGSYPGDPSSRKLGSFLIQTQLQCSGASGAILLTSAAQNLLCLKLAREVGVVISNPWITWFKVASVPAFVSLLCTPLIIYKLYPPELKHTPEAPAAAAKKLERLGPITKNEWIMLGAMAFTVSLWVFGEAIGIASVVSAMIGLSTLLLLGVINWDDCLSDKSAWDSLTWFAVLIGMAGQLTNLGVVAWMSDCVAKLLQSLSLTWPASFIILQACYLLIHYLFASQTGHAGALYPPFLAMQIAAGVPGVLAALCLAFNNNLSGALAHYSGGPAALYYGAGYVDLRDMFRVGFVMALVQAIIWGGVGSFWWKFLGLY

SEQ ID NO: 49 - protein, 557 aa, Arabidopsis thaliana
MASLALSGSCSLAFPLKSRSLSLPRPPSSSLNLTKPLRSLDSRFSLLKSPLPVSLRRRSSTLVKASSTVASASSSPTPPLVPAPVPWQGAAIKPLLASIATGLILWFVPVPEGVTRNAWQLLAIFLATIVGIITQPLPLGAVALMGLGASVLTKTLTFAAAFSAFGDPIPWLIALAFFFARGFIKTGLGNRVAYQFVRLFGSSSLGLGYSLVFSEALLAPAIPSVSARAGGIFLPLVKSLCVACGSNVGDGTEHRLGSWLMLTCFQTSVISSSMFLTAMAANPLSANLAFNTIKQTIGWTDWAKAAIVPGLVSLIVVPFLLYLIYPPTVKSSPDAPKLAQEKLDKMGPMSKNELIMAATLFLTVGLWIFGAKLGVDAVTAAILGLSVLLVTGVVTWKECLAESVAWDTLTWFAALIAMAGYLNKYGLIEWFSQTVVKFVGGLGLSWQLSFGILVLLYFYTHYFFASGAAHIGAMFTAFLSVSTALGTPPYFAALVLAFLSNLMGGLTHYGIGSAPIFYGANYVPLAKWWGYGFLISIVNILIWLGVGGAWWKFIGLW

SEQ ID NO: 50 - protein, 413 aa, Arabidopsis thaliana
MGLGASVLTKTLTFAAAFSAFGDPIPWLIALAFFFARGFIKTGLGNRVAYQFVRLFGSSSLGLGYSLVFSEALLAPAIPSVSARAGGIFLPLVKSLCVACGSNVGDGTEHRLGSWLMLTCFQTSVISSSMFLTAMAANPLSANLAFNTIKQTIGWTDWAKAAIVPGLVSLIVVPFLLYLIYPPTVKSSPDAPKLAQEKLDKMGPMSKNELIMAATLFLTVGLWIFGAKLGVDAVTAAILGLSVLLVTGVVTWKECLAESVAWDTLTWFAALIAMAGYLNKYGLIEWFSQTVVKFVGGLGLSWQLSFGILVLLYFYTHYFFASGAAHIGAMFTAFLSVSTALGTPPYFAALVHAFLSNLMGGLTHYGIGSAPIFYGANYVPLAKWWGYGFLISIVNILIWLGVGGAWWKFIGLW

SEQ ID NO: 51 - protein, 557 aa, Glycine max
MESYALHSLSTSFSRFSQLHHPPPPVISRSQFNQSQSLSLRSPITISQRFSFPSSTFKFNPFSKPHYPIQASPSSPPPPPPPTPIQGAKPIPFIISISIGLIVRFLVPKPVQVTPEAWQLLSIFLSTIAGLVLSPLPVGAWAFLGLTASVVTKTLTFTEAFGAFTNEVIWLIVISFFFARGFVKTGLGDRIATFFVKWMGKSTLGLSYGLTFSEVLIAPAMPSTTARAGGVFLPIIKSLSLSAGSEPASPTSKKLGAYLIQNQFQSAGNSSALFLTAAAQNLLCLKLAEELGVIVPNPWVTWFKAASLPAIVCLLLTPLILYKLYPPVIKDTPEAPALAAKKLESMGPVTKNEWIMVGTMLLAVSLWIFGDTIGIASAVAAMIGLSILLLLGVLDWNDCLNEKSAWDTLAWFAILVGMASQLTNLGIVSWMSDCVADNLRSFSLSWPASLAVLQAAYFFIHYLFASQTGHVGALYSAFLAMHRAAGVPGILAALALGYNTNLFGAITHYSSGQAAVYYGAGYVDLPDIFKMGFIMAFINAIIWGGVGSVWWKFLGLY

SEQ ID NO: 52 - protein, 562 aa, Glycine max
MESFALSASTSFSRFSQLHHRPRPVISRSQSLRSPITISQRFSFPSKPSSTFNFNFKFNRFSKPHYPIQASPSSSSSPPPPPPPPPPPPLQGAKPIPFIISISIGLIVRFFVPKPVEVTPEAWQLLSIFLSTIAGLVLSPLPVGAWAFLGLTASVVTKTLTFTAAFSAFTNEVIWLIVISFFFARGFVKTGLGDRIATYFVKWMGKSTLGLSYGLTFSEVLIAPAMPSTTARAGGVFLPIIKSLSLSAGSEPASPTSKKLGAYLIQNQFQSAGNSSALFLTAAAQNLLCLKLAEELGVIVPNPWVTWFKAASLPAVVCLLLTPLILYKLYPPEIKDTPEAPALAAKKLESMGPVTKNEWIMVGTMLLAVSLWIFGDTIGIASAVAAMIGLSILLLLGVLDWNDCLNEKSAWDTLAWFAILVGMASQLTNLGIVNWMSDYVAHNLRSFSLSWPASLAVLQAAYFFIHYLFASQTGHVGALYSAFLAMHRAAGVPGVLAALALGYNTNLFGAITHYSSGQAAVYYGAGYVDLPDIFKMGFIMAFINAIIWGGVGSVWWKFLGLY

SEQ ID NO: 53 - protein, 560 aa, Glycine max
MASVALTTTALPYLRLRRNTAATLKPNRHSSISLSSLKPTLNASISSFPNFSLKKPSLVFNQKPSSLTVRASAASITPAPAPSSAPVQPWQGASIKPLIASIATGVILWFSPVPAGVNRNAWQLLAIFLGTIVGIITQPLPLGAVAILGLGVSVLTKTLPFAAAFSGFGDPIPWLIALAFFFAKGFIKTGLGNRVAYQFVKLFGSSSLGLGYSLVFSEALLAPAIPSVSARAGGIFLPLVKALCVACGSNAGDGTEHRLGAWLMLTCFQTSVITSAMFLTAMAANPLCATLTLNSINQTIGWLDWAKAAIVPGLASLVLVPLILYVIYPPTLKSSPDAPKLAKEKLEKMGPMTTNEKIMTATLFLTVGLWVFGGLLNVDAVSAAILGLSVLLVTGVVTWKECLAEGVAWDTLTWFAALIAMAGYLNKYGLISWFSQTVVKFVGGLGLSWQLSFGILVLLYFYSHYFFASGAAHIGAMFTAFLSVATALGTPPFFGAIVLSFLSNLMGGLTHYGIGSAPVFFGANYVPLAKWWGYGFLISIVNIIIWLGLGGVWWKFIGLW

SEQ ID NO: 54 - protein, 553 aa, Glycine max
MASVALTTTALPSLRLRRNTTATLKPNRHASISLKPNLHASISSFPNFSLKKPHLISTRKPSALTVRASAASITPAPAPVQPWQGAAIKPLIASIATGVILWFSPVPAGVNRNAWQLLAIFLGTIVGIITQPLPLGAVAILGLGVSVLTKTLPFAAAFSGFGDPIPWLIALAFFFAKGFIKTGLGNRVAYQFVKLFGSSSLGLGYSLVFSEALLAPAIPSVSARAGGIFLPLVKALCVACGSNAGDGTEHRLGAWLMLTCFQTSVITSAMFLTAMAANPLCATLTQNSINQTIGWLDWAKAAIVPGLASLVLVPLILYVIYPPTLKSSPDAPKLAKEKLEKMGPMTTNEKIMTATLFLTVGLWVFGGLLNIDAVSAAILGLSVLLVTGVVTWKECLAEGVAWDTLTWFAALIAMAGYLNKYGLISWFSQTVVKFVGGLGLSWQLSFGILVLLYFYSHYFFASGAAHIGAMFTAFLSVATALGTPPFFGAIVLSFLSNLMGGLTHYGIGSAPVFFGANYVPLAKWWGYGFLISIVNIIIWLGLGGVWWKFIGLW

SEQ ID NO: 55 - protein, 554 aa, Zea mays
MELHLATIAHRPPLPVPARGHHLRRRLHHLPAPLSFQNTYSPSLSSPHHHRLSPTLRRHLRLPLLASQAPNSNPEPEPEPEPTGAKLLPLVISIAIGLAVRFLAPRPVEVSPQAWQLLSIFLSTIAGLVLGPLPVGAWAFLGLTAAVATRTLPFTAAFSAFTNEVIWLIVISFFFARGFVKTGLGDRIATYFVKWLGSSTLGLSYGLTLSEACIAPAMPSTTARAGGVFLPIIKSLSLSAESKPNHPSSRKLGSYLVMTQFQASGNSSALFLTAAAQNLLCLKLAEELGVIIANPWVSWFKAASLPALVSLLATPYLLYKIFPPETKDTPDAPALAEKKLKLMGPVTKNESVMIGTMILAVSLWVFGDAIGVSSVVAAMLGLSILLVLGVLDWDDCLNEKSAWDTLAWFAVLVGMAGQLTNLGIVSWMSNCVAKLLQSFSLSWPVAFCVLEASYFLIHYLFASQTGHVGALYSAFLAMHIAAGVPSVLSALALAFNTNLFGAITHYSSGQAAVYFGAGYMELPDVFRLGFITALANTLIWGVVGTIWWKFLGLY

SEQ ID NO: 56 - protein, 551 aa, Zea mays
MESLRLAVTHRPALPLPTSHSHLRRRHLHLHLHSYPNPLSLSPPIASHLSPIPRRHLPPLLASASASQASSKPAASAASGGAKPLPLFLSLAAGLAVRFLVPRPAEVTPEAWQLLSIFLSTIAGLVLGPLPVGAWAFLGLTATVATRTLPFTAAFGAFTNEVIWLIVISFFFARGFVKTGLGDRVATYFVKWLGRSTLGLSYGLAISEALISPAMPSTTARAGGVFLPIVKSLSLSSGSKPNDPSAKKLGSYLVQSQLQAAANSSALFLTAAAQNLLCLKLAEEIGVSIGNPWITWFKVSSVPAVLGLLVTPYLIYKIFPPEIKDTPEAPALAAEKLKNMGPVTKNEWVMIGTMLLAVSLWIFGETIGVSSVVAAMIGLSILLVLGVLNWEDCLNEKSAWDTLAWFAILVGLAGQLTNLGIVSWMSNCVAKVLQSFSLSWPAAFVVLQASYFFIHYIFASQTAHVGALYSAFLAMHLAAGVPALMSALALAYNANLFGALTHYSSGQSAVYFGAGYVDLPDVFKLGFITAAINAVIWGVAGALWWKFLGLY

SEQ ID NO: 57 - protein, 578 aa, Zea mays
MASSTAASPLTCHHLGSVGARPRLPSLSISLRRRSSSSSKPTSLSHSLPSKHSLAPPPAASASSRRGLTPVPASASAAAAPAPDPVPVPAPAPAPAPAPAAPPKKPALQGAAIKPLLASIATGVLIWLIPPPAGVPRNAWQLLAIFLSTIVGIITQPLPLGAVALLGLGAAVLSRTLTFAAAFSAFGDPIPWLIALAFFFARGFIKTGLGSRVAYAFVAAFGSSSLGLGYSLVFAEALLAPAIPSVSARAGGIFLPLVKSLCEACGSRAGDGTERRLGAWLMLTCFQTSVVSSAMFLTAMAANPLSANLTAATIGEGIGWTLWAKAAIVPGLLSLVLVPLILYVIYPPEVKASPDAPRLAKERLAKMGPMSKEETIMAGTLLLTVGLWIFGGMLNVDAVSAAILGLAVLLISGVVTWKECLAESVAWDTLTWFAALIAMAGYLNKFGLISWFSETVVKFVGGLGMSWQLSFGVLVLLYFYSHYFFASGAAHIGAMFTAFLSVASALGTPSLFAAMVLSFLSNLMGGTTHYGIGSAPVFYGAGYVPLAQWWGYGFVISVVNIIIWLGVGGFWWKIIGLW

SEQ ID NO: 58 - protein, 555 aa, Oryza sativa
MERLRVAISHHRAALPLPTHHNHFRRRHLQLQPFPSSLSLSLPISPQLSPAPPRRHLLPPLLASASAAQAAGPAPARAAGGGGGGAKPVPLLVSLAVGLAVRFLAPRPAEVTPQAWQLLSIFLTTIAGLVLGPLPVGAWAFLGLTATVATRTLPFTAAFGAFTNEVIWLIVISFFFARGFVKTGLGDRVATYFVKWLGRSTLGLSYGLAISEACIAPAMPSTTARAGGVFLPIVKSLSLSAGSKPNDPSARKLGSYLVQSQLQASGNSSALFLTAAAQNLLCLKLAEEIGVKIANPWISWFKVASLPAIISLLATPYLLYKIFPPEIKDTPEAPAIAAQKLKNMGPVTRNEWIMVATMILAVSLWIFGDTIGVSSVVAAMIGLSILLLLGVLNWEDCLNEKSAWDTLAWFAILVGMAGQLTNLGIVSWMSNCVAKVLQSFSLSWPAAFGVLQASYFFIHYLFASQTAHVGALYSAFLAMHLAAGVPAILSALALTYNSNLFGALTHYSSGQSAVYYGAGYVDLPDVFKLGFTTAAINAVIWGVVGTFWWKFLGLY

SEQ ID NO: 59 - protein, 550 aa, Oryza sativa
MESLRIAASHRPPLLLPSPHQLRRRHIAAVSLSLPHTSLSLSSHHHHHHRLAPTPLRRRIPPLLASQTPNPEADSPAPAGTKLAPLLVSLAVGLAVRFLAPRPPEVSPQAWQLLSIFLSTIAGLVLGPLPVGAWAFLGLTAAVATHTLPFAAAFSAFTNEVIWLIVISFFFARGFVKTGLGDRIATYFVKWLGGSTLGLSYGLTISEAFISPAMPSTTARAGGVFLPIIKSLSLSAGSKPNHPSSRKLGSYLVMSQFQAAGNSSALFLTAAAQNLLCLKLAEELGIIVANPWVAWFKAASLPAIASLLATPYLLYKIFPPETKDTPDAPALAAEKLERMGPVTKNEWVMIGTMLLAVSLWVFGDAIGVSSVVAAMLGLSILLLLGVLDWDDCLNEKSAWDTLAWFAVLVGMAGQLTNLGIVSWMSSCVAKLLESFSLSWPAAFCVLEASYFLIHYLFASQTGHVGALYSAFLAMHVAAGVPRVLSALALAFNTNLFGALTHYSSGQAAVYFGAGYLELPDVFRMGFVTALINILIWGVVGTFWWKLLGLY

SEQ ID NO: 60 - protein, 481 aa, Oryza sativa
MEQASCDYPTSAGARGHVRIVAIGRMQRVQIANGTCENHPDRQSFIVRQGTREHVYLGATRVALPLAAADAFGATATPSLGPSPALGSASVLGPLPVGAWAFLGLTAAVATHTLPFAAAFSAFTNEVIWLIVISFFFARGFVKTGLGDRIATYFVKWLGGSTLGLSYGLTISEAFISPAMPSTTARAGGVFLPIIKSLSLSAGSKPNHPSSRKLGSYLVMSQFQHGSKLLVCQPLLLKLATPYWFYKNFPLETRDTPDAPALAAEKLERMGPVTKNEWVMIGTMLLAVSLWVFGDAIGVSSVVAAMLGLSILLLLGVLDWDDCLNEKSAWDTLAWFAVLVGMAGQLTNLGIVSWMSSCVAKLLESFSLSWPAAFCVLEASYFLIHYLFASQTGHVGALYSAFLAMHVAAGVPRVLSALALAFNTNLFGALTHYSSGQAAVYFGAGYLELPDVFRMGFVTALINILIWGVVGTFWWKLLGLY

SEQ ID NO: 61 - protein, 548 aa, Oryza sativa
MATSTSAATAPLTCHHLGLRLRPRLPSLPLRPLSPSPSLSLSRPTPLTPSPPPRHRALHASASAAPAAPPSQPPKPVLQGAAIKPLVATIGTGVLIWLVPPPAGVARNAWQLLSIFLATIVGIITQPLPLGAVALLGLGAAVLTRTLTFAAAFSAFGDPIPWLIALAFFFARGFIKTGLGSRVAYAFVSAFGGSSLGLGYALVFAEALLAPAIPSVSARAGGIFLPLVKSLCEACGSRAGDGTERRLGSWLMLTCFQTSVISSAMFLTAMAANPLAANLTAGTIGQGIGWTLWAKAAIVPGLLSLVFVPLILYLIYPPEVKTSPDAPRLAKERLEKMGPMSKEEKIMAGTLFLTVGLWIFGGMLNVDAVSAAILGLSVLLISGVVTWKECLGEAVAWDTLTWFAALIAMAGYLNKYGLISWFSETVVKFVGGLGLSWQLSFGVLVLLYFYSHYFFASGAAHIGAMFTAFLSVSSALGTPPLIAAMVLSFLSNIMGGLTHYGIGSAPVFYGAGYVPLAQWWGYGFVISIVNIIIWLGAGGFWWKMLGLW

SEQ ID NO: 62 - protein, 556 aa, Triticum aestivum
MERLHLAVSHRPALPLPTPHNHLRRRHLQLQPSTSSLSLCRPISPHISLAPRRHLHPLLASASATQASSPNTEPAPAPAPAAASSGAKLLPLIASIAVGLAVRFLAPRPPEVTPQAWQLLSIFLSTIAGLVLGPLPVGAWAFLGLTATVATGTLPFTAAFGAFTNEVIWLIVISFFFARGFVKTGLGDRVATYFVKWLGGSTLGLSYGLTISEACIAPAMPSTTARAGGVFLPIVKSLSLSAGSKPNDPSAKKLGAYLVQSQLQASGNSSALFLTAAAQNLLCLKLAEEAGVKIASPWISWFKVASLPAIISLLATPYLLYKIFPPEIKDTPEAPALAAQKLKKMGPVTKNEWVMVATMILAVSLWIFGDAIGVSSVVAAMIGLSILLLLGVMNWDDCLNEKSAWDTLAWFAILVGMAGQLTDLGIVSWMSNCVAKVLQSFSLSWPAAFGVLQASYFFIHYLFASQTAHVGALYSAFLAMHLAAGVPAILSALALTYNSNLFGALTHYSSGQSAVYYGAGYVDLPDVFKLGFTAAAINALIWGVVGTFWWKFLGLY

SEQ ID NO: 63 - protein, 559 aa, Triticum aestivum
MASSASAASPLTCHHLGIRHRPHLPSFSLRRRPTSPLSSKPISLSLSHSHSHSLPKPLTPSTARHLLPPVAAAPASPPAPVSPPAKPALQGAAIKPLLASIATGVIIWFIPAPAGVARNAGQLLAVFLATIVGIITQPLPLGAVALLGLGAAVLTRTLTFAAAFSAFGDPIPWLIALAFFFARGFIKTGLGNRVAYQFVKAFGGSTLGLGYSLVFAEAFLAPAIPSVSARAGGIFLPLVKSLCEACGSRTDDGTERKLGAWLMLTCFQTSVVSSAMFLTAMAANPLAANLTLSTIGQGIGWTLWAKAAIVPGLLSLLIVPLVLYVIYPPEVETSPDAPRLAKERLAKMGPMSTEEKIMAGTLLLTVGLWIFGGMLSVDAVSAAILGLSVLLITGVVTWKECLAESVAWDTLTWFAALIAMAGYLNKYGLISWFSETVVKFVGGLGLSWQLSFGVLVLMYFYSHYFFASGAAHIGAMFTAFLSVASALGTPPLFAAMVMSFLSNLMGGLTHYGIGSAPVFYGAGYVPLAEWWGYGFVISVVNIIIWLGAGGFWWKMIGLW

SEQ ID NO: 64 - protein, 551 aa, Sorghum bicolor
MASLRLAVTHCPALPLPTPHSHLRRRQLQLHPYPNPLSLSPRISSHLSPIPRRHLPPLFASASASQAETKPPPPPTEASGGAKPLPLLISLAAGLAVRFLVPRPAEVTPEAWQLLSIFLSTIAGLVLGPLPVGAWAFLGLTATVATRTLPFTAAFGAFTNEVIWLIVISFFFARGFVKTGLGDRVATYFVKWLGRSTLGLSYGLAISEAFISPAMPSTTARAGGVFLPIVKSLSLSSGSKPNDPSAKKLGSYLVQSQLQAAANSSALFLTAAAQNLLCLKLAEEIGVNIGNPWITWFKVASVPALLGLLVTPYLIYKIFPPEIKDTPEAPALAAEKLKLMGPVTKNEWVMIATMLLAVSLWIFGEAIGVSSVVAAMIGLSILLLLGVLNWEDCLNEKSAWDTLAWFAILVGLAGQLTNLGIVSWMSNCVAKVLQSFSLSWPAAFGVLQASYFLIHYIFASQTAHVGALYSAFLAMHLAAGVPAVMSALALAYNANLFGALTHYSSGQSAVYFGAGYVDLPDVFKLGFITAALNAVVWGVAGAFWWKFLGLY

SEQ ID NO: 65 - protein, 557 aa, Sorghum bicolor
MENLHLAIAHRPPLPVPAAGHLRRRHLHLHHLPAPLSLPSTSHSLSSPHHHRLTPTLRRHLRPPLRVSQTPDANPEPEPEPESEPTGAKLVPFVISVAVGLAVRFLAPRPVEVSPQAWQLLSIFLSTIAGLVLGPLPVGAWAFLGLTAAVATRTLPFAAAFSAFTNEVIWLIVISFFFARGFVKTGLGDRIATYFVKWLGSSTLGLSYGLTISEACIAPAMPSTTARAGGVFLPIIKSLSLSAESKPNHPSSRKLGSYLVMTQFQASGNSSALFLTAAAQNLLCLKLAEELGVFIANPWVSWFKAASLPALAALLATPYLLYKIFPPETKDTPDAPALAEEKLKRMGPVTKNEWVMIGTMILAVSLWVFGDAIGVPSVVAAMLGLSILLLLGVLDWDDCLNEKSAWDTLAWFAVLVGMAAQLTNLGIVSWMSSCVAKLLQSFSLSWPVAFCILEGSYFLIHYLFASQTGHVGALYSAFLAMHIAAGVPRALSALALAFNTNLFGAITHYSSGQAAVYFGAGYIELPDVFRLGFITALINTFIWGVVGTIWWKFLGLY

SEQ ID NO: 66 - protein, 563 aa, Sorghum bicolor
MESSIRLADTLRPSSLPAPASAHLRRRHLYLHRLPRTSSSSSLFFSPSHHHRLCPTPRHDLLQPLAAAASGAAKLVPASPAPADSSPEPKPSGAKLVPLVISLAVGLAVRFLAPRPAEVSPRAWQLLSIFLSTIAGLVLGPLPVGAWAFLGLTAAVATHTLPFAAAFAAFTNEIIWLIVISFFFARGFVKTGLGDRVATYFVKWLGKSTLGLSYGLALGEACIAPAMPSTAARAGGIFLPIIKSLSLSAGSKPNHPSSRKLGTYLVMSQFQAASSSSALFLTAGAQNLLCLNLAEKFGVIIANPWVTWFKAASLPAIVSLLATPYLLYKIFPPEIKDTPEAPALAAEKQKQMGPVTKNEWAMIGTMILAVALWIFGDAIGVSSVVAAMLGLSILLLLGVLDWADILNEKSAWDTLAWFSVLVGMAAQLTSLGIVSWMSSCIANLLQSFSLSWPAAFCVLQASYLVIHYLFASQTGHVGALYSAFLAMHVAAGVPSVLSALALAFNTDLFGGITHYSSGQAAVYFGAGYLDLPDVFRIGFISTLINTLIWGGIGTFWWKFLGLY

SEQ ID NO: 67 - protein, 528 aa, Sorghum bicolor
MKSSHPTVATGDIEIGTTTTTTAETVVVVEADPPGCPASSATPSPKPAPAPAPAASGGAKPLPLFISLALGLAVRFLVPRPAQVTSQAWQLLSIFLSTIAGLVLAPLPVGAWAFLGLTVTVATRTLSFAAAFGAFTNEVIWLIVISFFFARGFVKTGLGDRVATYFVKWLGRSTLGLSYGLVISEAVISPAMPSTTARAGGVFLPIIKSLSLSSGSKPNDPSAKKLGSYLVQSQLQAAANSSALFLTAAAQNLLCLKLAEEIGVNIGNYWFTWFKVASVPALLGILVTPYLIYKIFPPEIKDTPEAPALAAEKLKNMGPVTKNEWAMIATMLLAVSLWIFGQTIGVSSVVASMIGLSILLLLGVLNWEDCLNEKSAWDTLAWFAILVGLAGQLTKLGIVSWISSSVAKILRSFSLSWPAAFGVLQASFFFIHYIFASQTAHVGALYSAFLAMHLAADVPAVMSTLALAYNANLFGSLTHYSSGQSAVYFGAGYVGLGDVFKLGFITAVINAVIWGAAGALWWKLLGLY

SEQ ID NO: 68 - protein, 584 aa, Sorghum bicolor
MASSTAASPLTCHHLGSVGARPSLPSLSFGPLRRRSSSKPISLSHSLPSKPSSLAPPPAASSSASASSSSRRGLTPVSASASAAAAPAPDPVPAPAPAPAPAPAPAAPPKKPALQGAAIKPLLASLAIGVLIWFLPAPAGVPRNAWQLLAIFLSTIVGIITQPLPLGAVALLGLGAAVLTKTLTFAAAFSAFGDPIPWLIALAFFFARGFIKTGLGSRVAYAFVAAFGSSSLGLGYSLVFAEALLAPAIPSVSARAGGIFLPLVKSLCEACGSRAGDGTERKLGAWLMLTCFQTSVVSSAMFLTAMAANPLSANLTAATIGQGIGWTLWAKAAIVPGLLSLVLVPLILYVIYPPEVKASPDAPRLAKERLAKMGPMSTEEKIMAGTLLLTVGLWIFGGMLSVDAVSAAILGLGVLLITGVVTWKECLAESVAWDTLTWFAALIAMAGYLNKYGFISWFSETVVKFVGGLGLSWQASFGVLVLLYFYSHYFFASGAAHIGAMFAAFLSVASALGTPSLFAAMVLSFLSNLMGGTTHYGIGSAPVFYGAGYVPLAQWWGYGFVISIVNIIIWLGAGGFWWKMIGLW

SEQ ID NO: 69 - protein, 584 aa, Solanum tuberosum
MERLALHSPSSATSAAAAATTSFSRLSYHHLRSRSSAIPTAALRPVSSLRSSISGSRFNLTGPRFNLFHPKPFLFNPLSKPTSRNPPSPKPITASSSPESDKIVIVDVKPKPQGAKLIPLIISVSIGLIVRFLVPRPPEVSPQAWQLLSIFLSTIAGLVLSPLPVGAWAFLGLTTSVLTKTLTFSSAFSAFTNEVIWLIVISFFFARGFVKTGLGDRIATYFVKWLGKSTLGLSYGLTLAEALIAPAMPSTTARAGGVFLPIIKSLSLSAGSKPGDPSSRKLGSYLIQSQFQCAGNSSALFLTAAAQNLLCLKLAEELGVVIANPWVSWFKAASLPAFISLLATPFILYKLYPPETKDTPEAPAMAAKKLELMGPVTKNEWVMIGTMLLAVSLWVFGDALGIASVVAAMLGLSILLLLGVLDWDDCLSEKSAWDTLAWFAVLVGMASQLTNLGIVGWMSSCVAKSLQALSLSWPAAFGVLQVSYFCIHYLFASQTGHVGALYSAFLAMHLASGVPGVLSALALAYNTNLFGALTHYSSGQAAVYFGAGYVDLPDVFKMGFVMAVVNAIIWGVVGTFWWKFLGLY

SEQ ID NO: 70 - protein, 508 aa, Solanum tuberosum
MSTPKSPLPTHNSTPSSPNHSPPPPPPSRPRRLPPWKGAKLIPLAISIAIGLIFRFAVPKPHKVTTNAWQLLAIFLATISGLVLGPLPVGAWAFLCLTVTVVTKTLTFAAAFAAFTNEVIWLIVVSFFFSRGFIKTGLGDRIALCFVSWLGKNTLGLSYGLALSEAAISPAIPSTTARAGGIFLPIIKSLAVTADSHPKNDSSRKLGAYLIQSQLQCSSSSSALFLTAAAQNLLCVKLAEGLGVQVSSKWLTWLKASCIPAVISLLVTPVVLYKIFPPEMKDTPDAPLMARRRLQQMGPMKSDEWVMTIVMLVTVGLWIAGEAIGLASVITAMLGLALLLTFGILDWNDCLSEKSAWDTLAWFGVLIGMASQLTTLGVVAWMSNAVGNYLESLSLHWFGAFCILQAAYFFIHYLFASQTGHVAALYSAFLAMCLAAKVPGLFAALALGYNTNLFGGLTHYSSGQAAVYYGAGYVELRDVFKLGIIIAIMNIVIWAVAGAGWWKVLGLY

SEQ ID NO: 71 - protein, 554 aa, Solanum tuberosum
MASLALTSAVNLRLRPTPSQKPRISITQSLHFTQTSLKTTNLGKSLNLGGKRLNHEKSRRVIVKTSASASASSPAIVPQQQPPWQGAAMKPLIASIATGVILWFIPAPAGVTKNAWQLLAIFLATIVGIITQPLPLGAVALMGLGACVLTKTLTFAAAFSAFGDPIPWLIALAFFFARGFIKTGLGNRIAYQFVKLFGSSSLGLGYSLVFSEALLAPAIPSVSARAGGIFLPLVKSLCVACGSNAGDGTEHKLGSWLMLTCFQTSVISSSMFLTAMAANPLSANLTLSTINQTIGWMDWAKAAIVPGLVSLIVVPLLLYIIYPPTVKSSPDAPRLAKERLEQMGPMSKNEIIMAGTLLLTVGLWVFGGALKVDAVTAAILGLSVLLVTGVVTWKECLGEAVAWDTLTWFAALIAMAGYLNKYGLISWFSETVVKVVGGLSLSWQLSFGILVLLYFYSHYFFASGAAHIGAMFTAFLSVASALGTPPYLGALVLSFLSNLMGGITHYGIGSAPVFYGANYVPLAKWWGYGFVCSVVNLIIWLGVGGIWWKAIGLW

SEQ ID NO: 72 - protein, 563 aa, Brassica napus
MESFALHSISTTAASFSHHPSRLSLLRRISSRSPPPTISLPSLRSHSVQPLTFPLLKPIPRLSARIAAAPRDNIPPPPPSQPSEPPSSQPPQGAKLLPLILSLSVGLILRFAVPLPEGLTPQGWQLLSIFLSTIAGLVLSPLPVGAWAFMGLTASIVTKTLSFSAAFSAFTSEVIWLIVISFFFARGFVKTGLGDRIATYFVKWLGKSTLGLSYGLTISEALIAPAMPSTTARAGGIFLPIIKSLSLSAGSKPGDSSSRKLGSYLIQNQFQCAGNSSALFLTAAAQNLLCLKLAEELGVVIANPWVSWFKAASLPAIISLLCTPLILYKLYPPETKDTPEAPGIAALKLKEMGPVTKNEWIMVGTMLLAVTLWICGESLGIPSVVAAMIGLSILLLLGVLNWDDCLSEKSAWDTLAWFAVLVGMAGQLTNLGVVTWMSDCVAKVLQSLSLSWPAAFGLLQAAYFFIHYLFASQTGHVGALFSAFLAMNIAAGVPGVLAALALAYNTNLFGALTHYSSGQAAVYYGAGYVDLPDVFKIGFVMATINAIIWGVVGTFWWKFLGLY

SEQ ID NO: 73 - protein, 556 aa, Brassica napus
MESYALHSPSTPASFSHHPSRLSLLRRISSRSPPSTISLPSLRSHSVQPLTFPLLKPIPRLSARIAAAPRDDIPPPSPSPSQPQQGAKLLPLILSLSVGLILRFAVSLPEGLTPQGWQLLSIFLSTIAGLVLSPLPVGAWAFMGLTASIVTKTLSFSAAFSAFTSEVIWLIVISFFFARGFVKTGLGDRIATYFVKWLGKSTLGLSYGLTISEALIAPAMPSTTARAGGIFLPIIKSLSLSAGSKPGDSSSRKLGSYLIQSQFQCAGNSSALFLTAAAQNLLCLKLAEELGVVIANPWVSWFKAASLPAIISLLCTPLILYKLYPPETKDTPEAPGIAALKLKEMGPVTKNEWIMVGTMLLAVTLWICGESLGIPSVVAAMIGLSILLLLGVLNWDDCLSEKSAWDTLAWFAVLVGMAGQLTNLGVVTWMSDCVAKVLQSLSLSWPAAFGLLQAAYFFIHYLFASQTGHVGALFSAFLAMNIAAGVPGVLAALALAYNTNLFGALTHYSSGQAAVYYGAGYVDLPDVFKIGFVMATINAIIWGVVGTFWWKFLGLY

SEQ ID NO: 74 - protein, 555 aa, Brassica napus
MESFALHSLSTTATSASFSHHPSRQSLLRRISSRSPPSSISLRSHSVKPLAFPLLKPIHRFSTRIAAAPRDDSPPPPPSPQPPQGAKLVPLILSLSVGLILRFAVSVPEGVTPQGWQLLSIFLATIAGLVLSPLPVGAWAFIGLTASIVTKTLSFSAAFSAFTSEVIWLIVISFFFARGFVKTGLGDRIATYFVKWLGKSTLGLSYGLTLSEALIAPAMPSTTARAGGIFLPIIKSLSLSAGSKPGDPSSRKLGSYLIQSQFQCAGNSSALFLTAAAQNLLCLKLAEELGVVIANPWVSWFKAASLPAIISLLCTPLILYKLYPPETKDTPDAPGIAALKLKQMGPVTKNEWIMVGTMVLAVTLWICGETLGIPSVVAAMIGLSILLLLGVLNWDDCLSEKSAWDTLAWFAVLVGMAGQLTNLGVVSWMSDCVAKALQSLSLSWPAAFGLLQAAYFFIHYLFASQTGHVGALFSAFLAMNIAAGVPGILAALALAYNTNLFGALTHYSSGQAAVYYGAGYVDLPDVFKIGFVMATINAIIWGVVGTFWWKFLGLY

SEQ ID NO: 75 - protein, 562 aa, Brassica napus
MESFALHSLSTTATSASFSHHPSRQSLLRRISSRSPPSSISLRSHSVKPLALPLLKPIHRFCTRIAAAPRDDSPPPPPTPSSESPSPQPPQGAKLVPLILSLSVGLILRFAVSVPEGVTPQGWQLLSIFLSTIAGLVLSPLPVGAWAFIGLTASIVTRTLSFSAAFSAFTSEVIWLIVISFFFARGFVKTGLGDRIATYFVKWLGKSTLGLSYGLTLSEALIAPAMPSTTARAGGIFLPIIKSLSLSAGSKPGDPSSRKLGSYLIQSQFQCAGNSSALFLTAAAQNLLCLKLAEELGVVIANPWVSWFKAASLPAIISLLCTPLILYKLYPPETKDTPDAPGIAALKLKQMGPVTKNEWIMVGTMLLAVTLWICGETLGIPSVVAAMIGLSILLLLGVLNWDDCLSEKSAWDTLAWFAVLVGMAGQLTNLGVVSWMSDCVAKALQSLSLSWPAAFCLLQAAYFFIHYLFASQTGHVGALFSAFLAMNIAAGVPGILAALALAYNTNLFGALTHYSSGQAAVYYGAGYVDLPDVFKIGFVMAAINAIIWGVVGTFWWKFLGLY

SEQ ID NO: 76 - protein, 484 aa, Brassica napus
MESFALHSLSTTATSASFSHHPSRQPLLRRISSRSPPSSISLRSHSVKPLALPLLKPIHRFCTRIAAAPRDDSPPPPPTPSSESPSPQPPQGAKLVPLILSLSVGLILRFAVSVPEGVTPQGWQLLSIFLSTIAGLVLSPLPVGAWAFIGLTASIVTRTLSFSAAFSAFTSEVIWLIVISFFFARGFVKTGLGDRIATYFVKWLGKSTLGLSYGLTLSEALIAPAMPSTTARAGGIFLPIIKSLSLSAGSKPGDPSSRKLGSYLIQSQFQCAGNSSALFLTAAAQNLLCLKLAEELGVVIANPWVSWFKAASLPAIISLLCTPLILYKLYPPETKDTPDAPGIAALKLKQMGPVTKNEWIMVGTMVLAVTLWICGETLGIPSVVAAMIGLSILLLLGVLNWDDCLSEKSAWDTLAWFAVLVGMAGQLTNLGVVSWMSDCVAKALQSLNLSWPDAFGLLQAAHFFIHYLFAKPNRSRKSSLLSIS

SEQ ID NO: 77 - protein, 657 aa, Brassica napus
MSDCVAKALQSLNLSWPDAFGFLQAAYFLTHYPFASQTGHVRALFSAYLSYNKIRCFISFTCCSFQHQSFCCFDAYSSGQVPNKLNLPRRELRINSTTMESFALRSLSTTATSSLSYLSLRRSSSRSLSLSLTHPPISLYTSSPTVRSLSTSSPRLTLRATASSASSSPEIQNNPQSSSSSSSPPQGAKLIPVAISISIGLIVRFLIPKPEQVTPQGWQLLSVFLFTISGLVLGPLPVGAWAFIGLTASIVTRTLPFSTAFAAFTNELIWLIAISFFFARGFVKTGLGDRIATYFVKWLGRSTLGLSYGLVLCETFMGLIMPSTMARAGGVFLPVIKSLSLSAGSRPGDSSSRKLGAFLIQTQLQCAGTSGALLLTSAAQNLLCLKLAREVGVVLSNPWVSWFKAASVPAFASLLCTPLLIYKLYPPELKHTPEAPAAAAKKLERLGPITKNEWIMLGAMAFTVSLWVFGEAIGVSSVVSAMIGLSMLLLLGVINWNDCLSDKSAWDSLTWFAVLIGMAGQLTNLGVVSWMSDCVAKLLQTLSLTWPASFVILQASYLLLHYVFASQTAHAGALYPAFLAMQIAAGVPGVLAALCLAFNNNLSGALAHYSSGPAALYYGAGYVDLKDMFRLGFVMALLQAVIWGSVGSVWWKFLGLY

SEQ ID NO: 78 - protein, 559 aa, Brassica napus
MESFALRSLSTTASSPLSYLSLRRSSSRSLSLSLTHPSISLYTSSPTLRSLSISSPRLTLRATASSTSSSPEIQSNPQSSSSSSSPPQGAKLIPLAISISIGLIVRFLIPRPEQVTSQGWQLLSVFLFTISGLVLGPLPVGAWAFIGLTASIVTRTLSFSTAFAAFTNELIWLIAISFFFARGFVKTGLGDRIATYFVKWLGRSTLGLSYGLVLCETFMGLIMPSTMARAGGVFLPVIKSLSLSAGSKPGDSSSRRLGAFLIQTQLQCAGTSGALLLTSAAQNLLCLKLAREVGVVLSNPWVSWFKAASVPAFASLLCTPLIIYKLYPPELKHTPEAPAAAAKKLERLGPITKNEWVMLGAMAFTVSLWIFGEAIGVSSVVSAMIGLSTLLVLGVINWNDCLSDKSAWDSLTWFAVLIGMAGQLTNLGVVAWMSDCVAKLLQTLSLTWPASFVILQASYLLLHYVFASQTAHAGALYPAFLAMQIAAGVPGVLAALCLAFNNNLSGALAHYSSGPAALYYGAGYVDLKDMFRLGFVMALLQAVIWGSVGSLWWKFLGLY

SEQ ID NO: 79 - protein, 559 aa, Brassica napus
MESFALRSLSTTASSPLSYLSLRRSSSRSLSLSITHPSISLYTSSSTVRSLSTSSPRLTLRATASSTSSSPEIQNNPQSSSSSSSPPQGAKLVPLAISISIGLIVRFLIPRPEQVTSQGWQLLSVFLFTISGLVLGPLPVGAWAFIGLTASIVTRTLPFSTAFAAFTNELIWLIAISFFFARGFVKTGLGDRIATYFVKWLGRSTLGLSYGLVLCETFMGLIMPSTMARAGGVFLPVIKSLSLSAGSRPGDSSSRKLGAFLIQTQLQCAGTSGALLLTSAAQNLLCLKLAREVGVVLSNPWVSWFKAASVPAFASLLCTPLIIYKLYPPELKHTPEAPAAAAKKLERLGPITKNEWIMLSAMAFTVSLWVFGEAIGVSSVVSAMIGLSMLLLLGVINWNDCLSDKSAWDSLTWFAVLIGMAGQLTNLGVVSWMSDCAAKLLQTLSLTWPASFVILQASYLLLHYVFASQTAHAGALYPAFLAMQIAAGVPGVLAALCLAFNNNLSGALAHYSSGPAALYYGAGYVDLKDMFRLGFVVALLQAVIWGSVGSLWWKFLGLY

SEQ ID NO: 80 - protein, 552 aa, Brassica napus
  misc_feature (391)..(394): Xaa can be any naturally occurring amino acid
MASLALSGSCSLAFPLKSRPLLLPRPPSSLNLLKKPLRSTESRFSSVKSPLHIALTKRSTLVKASSSASSPAPVAPAPWQGAAIKPLLASIATGVIIWFLPVPEGVTRSAWQLLAIFLATIVGIITQPLPLGAVALLGLGASVLTKTLTFAAAFSAFGDPIPWLIALAFFFARGFIKTGLGNRVAYQFVRLFGSSSLGLGYSLVFSEALLAPAIPSVSARAGGIFLPLVKSLCVACGSNVGDGTEHRLGAWLMLTCFQTSVISSSMFLTAMAANPLSANLAFNTIKQTIGWTDWAKAAIVPGIVSLIVVPFLLYLIYPPTVKSSPDAPKLAQEKLDKMGPMSKNELIMAATLFLTVGLWIFGAKLSVDAVTAAILGLSVLLVTGVVTWKEXXXXLAESVAWDTLTWFAALIAMAGYLNKYGLIEWFSQTVVKFVGGLGLSWQLSFGILVLLYFYTHYFFASGAAHIGAMFTAFLSVSTALGTPPYFAALVLAFLSNLMGGLTHYGIGSAPIFYGANYVPLSKWWGYGFLISIVNILIWLGVGGAWWKFIGLW

(19) United States
(12) Patent Application Publication
(10) Pub. No.: US 2020/0140879 A1
(43) Pub. Date: May 7, 2020

(54) METHODS AND GENES FOR PRODUCING LAND PLANTS WITH INCREASED EXPRESSION OF MITOCHONDRIAL METABOLITE TRANSPORTER AND/OR PLASTIDIAL DICARBOXYLATE TRANSPORTER GENES

(71) Applicant: YIELD10 BIOSCIENCE, INC., Woburn, MA (US)

(72) Inventors: Frank Anthony SKRALY, Woburn, MA (US); Kristi D. SNELL, Woburn, MA (US)

(21) Appl. No.: 16/624,637

(22) PCT Filed: Jun. 22, 2018
(86) PCT No.: PCT/US2018/038927

§ 371 Date: Dec. 19, 2019

Related U.S. Application Data
(60) Provisional application No. 62/524,134, filed on Jun. 23, 2017.
(60) Provisional application No. 62/546,268, filed on Aug. 16, 2017.

(51) Int. Cl. C12N 15/82; C07K 14/415

(52) U.S. Cl. CPC C12N 15/8261; C12N 15/8213; C07K 14/415

(57) ABSTRACT

A land plant is disclosed. The land plant has increased expression of a mitochondrial transporter protein such that the flux of metabolites through the mitochondrial membrane is increased and the land plant has higher performance and/or yield as compared to a reference land plant not having the increased expression of the mitochondrial transporter protein. Another land plant also is disclosed. The land plant has increased expression of a plastidial dicarboxylate transporter protein such that the flux of metabolites through the plastidial membrane is increased and the land plant has higher performance and/or yield as compared to a reference land plant not having the increased expression of the plastidial dicarboxylate transporter protein.

### FIELD OF THE INVENTION

The present invention relates generally to methods, genes and systems for producing land plants with increased expression of mitochondrial metabolite transporter genes and/or proteins, and/or plastidial dicarboxylate transporter genes and/or proteins, and more particularly to such methods, genes and systems wherein flux of metabolites through the mitochondrial membrane and/or plastidial membrane is increased, resulting in increased crop performance and/or yield.

### BACKGROUND OF THE INVENTION

The world faces a major challenge in the next 35 years to meet the increased demands for food production to feed a growing global population, which is expected to reach 9 billion by the year 2050. Food output will need to be increased by up to 70% in view of the growing population, increased demand for improved diet, land use changes for new infrastructure, alternative uses for crops and changing weather patterns due to climate change. Studies have shown that traditional crop breeding alone will not be able to solve this problem (Deepak K. Ray, Nathaniel D. Mueller, Paul C. West and Jonathon A. Foley, 2013. Yield trends are Insufficient to Double Global Crop Production by 2050. PLOS, published Jun. 19, 2013 doi.org/10.1371/journal.pone.0066428). There is therefore a need to develop new technologies to enable step change improvements in crop performance and in particular crop productivity and/or yield.

Major agricultural crops include food crops, such as maize, wheat, oats, barley, soybean, millet, sorghum, pulses, bean, tomato, corn, rice, cassava, sugar beets, and potatoes, forage crop plants, such as hay, alfalfa, and silage corn, and oilseed crops, such as camelina, Brassica species (e.g. B. napus (canola), B. rapa, B. juncea, and B. carinata), crambe, soybean, sunflower, safflower, oil palm, flax, and cotton, among others. Productivity of these crops, and others, is limited by numerous factors, including for example relative inefficiency of photochemical conversion of light energy to fixed carbon during photosynthesis, as well as loss of fixed carbon by photorespiration and/or other essential metabolic pathways having enzymes catalyzing decarboxylation reactions. Crop productivity is also limited by the availability of water. Achieving step changes in crop yield requires new approaches.

One potential approach involves metabolic engineering of crop plants to express carbon-concentrating mechanisms of cyanobacteria or eukaryotic algae. Cyanobacteria and eukaryotic algae have evolved carbon-concentrating mechanisms to increase intracellular concentrations of dissolved inorganic carbon, particularly to increase concentrations of CO₂ at the active site of ribulose-1,5-bisphosphate carboxylase/oxygenase (also termed RuBisCO). It has recently been shown by Schnell et al., WO 2015/103074 that Camelina plants transformed to express CCP1 of the algal species Chlamydomonas reinhardtii have reduced transpiration rates, increased CO₂ assimilation rates and higher yield than control plants which do not express the CCP1 gene. More recently, Atkinson et al., (2015) Plant Biotechnol. J., doi: 10.1111/pbi. 12497, discloses that CCP1 and its homolog CCP2, which were previously characterized as Ci transporters, previously reported to be in the chloroplast envelope, localized to mitochondria in both Chlamydomonas reinhardtii, as expressed naturally, and tobacco, when expressed heterologously, suggesting that the model for the carbon-concentrating mechanism of eukaryotic algae needs to be expanded to include a role for mitochondria. Atkinson et al. (2015) disclosed that expression of individual Ci (bicarbonate) transporters did not enhance growth of the plant Arabidopsis.

In co-pending Patent Application PCT/US2017/016421, to Yield10 Bioscience, a number of orthologs of CCP1 from algal species that share common protein sequence domains including mitochondrial membrane domains and transporter protein domains were shown to increase seed yield and reduce seed size when expressed constitutively in Camelina plants. Schnell et al., WO 2015/103074, also reported a decrease in seed size in higher yielding Camelina lines expressing CCP1.

In U.S. Provisional Patent Application 62/462,074, to Yield10 Bioscience, CCP1 and its orthologs from other eukaryotic algae are referred to as mitochondrial transporter proteins. The inventors tested the impact of expressing CCP1 or its algal orthologs using seed-specific promoters with the unexpected outcome that both seed yield and seed size increased. These inventors also recognized the benefits of combining constitutive expression and seed specific expression of CCP1 or any of its orthologs in the same plant.

In co-pending application U.S. Provisional Patent Application 62/520,785, to Yield10 Bioscience, sequence and structural orthologs of CCP1 were identified in a select number of plant species for the first time and the inventors disclosed genetically engineered land plants that express plant CCP1-like mitochondrial transporter proteins.

Unfortunately, “transgenic plants,” “GMO crops,” and/or “biotech traits” are not widely accepted in some regions and countries and are subject to regulatory approval processes that are very time consuming and prohibitively expensive. The current regulatory framework for transgenic plants results in significant costs (˜$136 million per trait; McDougall, P. 2011, “The cost and time involved in the discovery, development, and authorization of a new plant biotechnology derived trait.” Crop Life International) and lengthy product development timelines that limit the number of technologies that are brought to market. This has severely impaired private investment and the adoption of innovation in this crucial sector. Recent advances in genome editing technologies provide an opportunity to precisely remove genes or edit control sequences to significantly improve plant productivity (Belhaj, K. 2013, Plant Methods, 9, 39; Khandagale & Nadal, 2016, Plant Biotechnol Rep, 10, 327) and open the way to produce plants that may benefit from an expedited regulatory path, or possibly unregulated status.

Given the costs and challenges associated with obtaining regulatory approval and societal acceptance of transgenic crops there is a need to identify, where possible, plant mitochondrial transporter proteins, ideally derived from crops or other land plants, that can be genetically engineered to enable enhanced carbon capture systems to improve crop yield and/or seed yield, particularly without relying on genes, control sequences, or proteins derived from non-land plants to the extent possible.

### BRIEF SUMMARY OF THE INVENTION

Methods, genes and systems for producing land plants with increased expression of mitochondrial metabolite transporter genes are disclosed. The land plants have increased expression of mitochondrial metabolite transporter genes such that the flux of metabolites through the mitochondrial membrane is increased, resulting in increased crop performance and/or yield. The genes encoding the mitochondrial metabolite transporter genes can be used alone or in combinations. The expression of the genes encoding the mitochondrial metabolite transporter proteins can be increased using genetic engineering techniques or marker assisted breeding approaches to develop plants with increased performance and/or yield. Where genetic engineering techniques are used to increase the expression of the mitochondrial metabolite transporter proteins, the increased expression can be accomplished using transgenic technologies with transporter genes from a source other than the plant being modified, by cis-genic approaches, by introducing additional copies of transporter genes from the same plant species or by genome editing approaches to increase the expression of the transporter genes in a constitutive or seed specific manner. In some examples, the land plants with increased expression of mitochondrial metabolite transporter genes also have increased expression of plastidial dicarboxylate transporter genes.

Similarly, methods, genes and systems for producing land plants with increased expression of plastidial dicarboxylate transporter genes also are disclosed. The land plants comprise increased expression of plastidial dicarboxylate transporter genes such that the flux of metabolites through the plastidial membrane is increased, resulting in increased crop performance and/or yield too. In some examples, the land plants with increased expression of plastidial dicarboxylate transporter genes also have increased expression of mitochondrial transporter genes.

As will be appreciated, increased expression of mitochondrial transporter genes or plastidial dicarboxylate transporter genes can result in increased expression of corresponding mitochondrial transporter proteins or plastidial dicarboxylate transporter proteins, respectively.

Accordingly, a land plant is provided. The land plant has increased expression of a mitochondrial transporter protein such that the flux of metabolites through the mitochondrial membrane is increased and the land plant has higher performance and/or yield as compared to a reference land plant not having the increased expression of the mitochondrial transporter protein.

In some examples, the mitochondrial transporter protein increases the flow of dicarboxylic acids through the mitochondrial membrane, resulting in the land plant having higher performance and/or yield.

In some examples, the mitochondrial transporter protein transports oxaloacetate into or out of the mitochondria of the land plant. In some of these examples, the mitochondrial transporter protein is an oxaloacetate shuttle that transports oxaloacetate through the mitochondrial membrane in one direction while simultaneously transporting another metabolite in the other direction. Also in some of these examples, the second metabolite is another dicarboxylic acid. Also in some of these examples, the other dicarboxylic acid is selected from one or more of malate, succinate, maleate, or malonate.

In some examples, the mitochondrial transporter protein comprises one or more of Arabidopsis thaliana DTC (SEQ ID NO: 1), DIC1 (SEQ ID NO: 2), DIC2 (SEQ ID NO: 3), or DIC3 (SEQ ID NO: 4). In some examples, the mitochondrial transporter protein comprises one or more orthologs of DTC in maize. In some examples, the mitochondrial transporter protein comprises one or more orthologs of DIC1 in maize. In some examples, the mitochondrial transporter protein comprises one or more orthologs of DTC in soybean. In some examples, the mitochondrial transporter protein comprises one or more orthologs of DIC1 in soybean. In some examples, the mitochondrial transporter protein comprises one or more orthologs of DTC in rice, wheat, sorghum, potato, or canola. In some examples, the mitochondrial transporter protein comprises one or more orthologs of DIC1 in rice, wheat, sorghum, potato, or canola.

In some examples, the land plant is a genetically engineered land plant, and the increased expression of the mitochondrial transporter protein is based on the genetic engineering.

In some examples, the land plant further has increased expression of a plastidial dicarboxylate transporter protein such that the flux of metabolites through the plastidial membrane is increased and the land plant has higher performance and/or yield as compared to a reference land plant not having the increased expression of the plastidial dicarboxylate transporter protein. In some of these examples, the increased expression of the plastidial dicarboxylate transporter protein is induced by the increased expression of the mitochondrial transporter protein. Also in some of these examples, the plastidial dicarboxylate transporter protein directs malate and/or oxaloacetate into and/or out of the chloroplasts of the land plant. Also in some of these examples, the plastidial dicarboxylate transporter protein comprises one or more of Camelina sativa Csa10909s010 (SEQ ID NO: 46), a homolog of Camelina sativa Csa10909s010, or an ortholog of Camelina sativa Csa10909s010. Also in some of these examples, the plastidial dicarboxylate transporter protein comprises one or more of a 2-oxoglutarate/malate transporter (OMT), a general dicarboxylate transporter (DCT), or an oxaloacetate transporter (OAT).

Another land plant also is provided. The land plant has increased expression of a plastidial dicarboxylate transporter protein such that the flux of metabolites through the plastidial membrane is increased and the land plant has higher performance and/or yield as compared to a reference land plant not having the increased expression of the plastidial dicarboxylate transporter protein.

In some examples, the land plant further has increased expression of a mitochondrial transporter protein such that the flux of metabolites through the mitochondrial membrane is increased and the land plant has higher performance and/or yield as compared to a reference land plant not having the increased expression of the mitochondrial transporter protein. In some of these examples, the increased expression of the mitochondrial transporter protein is induced by the increased expression of the plastidial dicarboxylate transporter protein.

In some examples, the plastidial dicarboxylate transporter protein comprises one or more of Camelina sativa Csa10909s010 (SEQ ID NO: 46), a homolog of Camelina sativa Csa10909s010, or an ortholog of Camelina sativa Csa10909s010. In some examples, the plastidial dicarboxylate transporter protein comprises one or more of a 2-oxoglutarate/malate transporter (OMT), a general dicarboxylate transporter (DCT), or an oxaloacetate transporter (OAT).

### BRIEF DESCRIPTION OF THE DRAWINGS

FIG. 1 shows pathways involved in photorespiration where RuBisCo fixes oxygen (reaction 2) instead of CO₂ (reaction 1), resulting in the production of 2PGc, which must be removed through a series of metabolic reactions occurring in the chloroplast, peroxisome, and mitochondrion. Intermediates transferred to and from the mitochondrion during this process are shown with dashed arrows and are candidates for novel transporters to increase the flow of carbon and prevent the buildup of intermediates that may inhibit plant productivity. Abbreviations are as follows. RuBisCo, ribulose-1,5-bisphosphate carboxylase/oxygenase; Ru15BP, ribulose 1,5-bisphosphate; 3PG, 3-phosphoglycerate; 2PGc, 2-phosphoglycolate; GOX, glyoxylate; Glu, glutamate; 2-OG, 2-oxoglutarate or alpha-ketoglutarate; Ser, serine; Gly, glycine; HPYR, hydroxypyruvate; OAA, oxaloacetate; MAL, malate.

FIG. 2 shows optimal mitochondrial metabolism with and without photorespiration (PR), based on the AraGEM model, using a basis of 100 photons and an objective function of maximum biomass.

FIG. 3 shows optimal mitochondrial metabolism with and without photorespiration (PR), based on the AraGEM model, using a basis of 100 photons and an objective function of maximum biomass, as in FIG. 2, but with 2-oxoglutarate import not permitted.

FIG. 4 shows optimal mitochondrial metabolism with and without photorespiration (PR), based on the AraGEM model, using a basis of 100 photons and an objective function of maximum biomass, as in FIG. 2, but using the set of mitochondrial transport functions prescribed by Cheung et al. (2013, Plant J. 75:1050-61).

FIG. 5A-B shows a multiple sequence alignment of DTC (SEQ ID NO: 1), DIC1 (SEQ ID NO: 2), DIC2 (SEQ ID NO: 3), and DIC3 (SEQ ID NO: 4) according to CLUSTAL O(1.2.4).

FIG. 6 shows binary transformation vector pYTEN-10 (SEQ ID NO: 5) for expressing the Arabidopsis DTC gene using the soybean oleosin seed specific promoter.

FIG. 7 shows binary transformation vector pYTEN-11 (SEQ ID NO: 6) for expressing the Arabidopsis DIC1 gene using the soybean oleosin seed specific promoter.

FIG. 8 shows binary transformation vector pYTEN-12 (SEQ ID NO: 7) for expressing the Arabidopsis DIC2 gene using the soybean oleosin seed specific promoter.

FIG. 9 shows binary transformation vector pYTEN-13 (SEQ ID NO: 8) for expressing the Arabidopsis DIC3 gene using the soybean oleosin seed specific promoter.

FIG. 10 shows binary transformation vector pYTEN-14 (SEQ ID NO: 9) for expressing the Arabidopsis DTC gene using the CaMV35 S-tetramer constitutive promoter.

FIG. 11 shows binary transformation vector pYTEN-15 (SEQ ID NO: 10) for expressing the Arabidopsis DIC1 gene using the CaMV35S-tetramer constitutive promoter.

FIG. 12 shows binary transformation vector pYTEN-16 (SEQ ID NO: 11) for expressing the Arabidopsis DIC2 gene using the CaMV35S-tetramer constitutive promoter.

FIG. 13 shows binary transformation vector pYTEN-17 (SEQ ID NO: 12) for expressing the Arabidopsis DIC3 gene using the CaMV35S-tetramer constitutive promoter.

FIG. 14 shows DNA fragment pYTEN-18 (SEQ ID NO: 13) for expressing the maize ortholog of the Arabidopsis DTC gene using the maize Cab5 promoter with an Hsp70 intron for expression of the gene in green tissue.

FIG. 15 shows DNA fragment pYTEN-19 (SEQ ID NO: 14) for expressing the maize ortholog of the Arabidopsis DTC gene using the A27znGlb1 chimeric promoter containing maize sequences for seed specific expression of the maize ortholog of the Arabidopsis DTC gene.

FIG. 16 shows DNA fragment pYTEN-20 (SEQ ID NO: 15) for expressing the maize ortholog of the Arabidopsis DIC1 gene using the maize Cab5 promoter with an Hsp70 intron for expression of the gene in green tissue.

FIG. 17 shows DNA fragment pYTEN-21 (SEQ ID NO: 16) for expressing the maize ortholog of the Arabidopsis DIC1 gene using the A27znGlb1 chimeric promoter containing maize sequences for seed specific expression of the maize ortholog of the Arabidopsis DTC gene.

FIG. 18 shows linear vector pYTEN-22 (SEQ ID NO: 17) for expressing the soybean ortholog of the Arabidopsis DTC gene using the soybean oleosin promoter. A cassette containing only the soybean promoter, the soybean ortholog of the Arabidopsis DTC gene, and the soybean oleosin terminator can be released by digestion with the Sma I restriction enzyme for introduction into soybean.

FIG. 19 shows linear vector pYTEN-23 (SEQ ID NO: 18) for expressing the soybean ortholog of the Arabidopsis DIC1 gene using the soybean oleosin promoter. A cassette containing only the soybean promoter, the soybean ortholog of the Arabidopsis DIC gene, and the soybean oleosin terminator can be released by digestion with the Spe I and Swa I restriction enzymes for introduction into soybean.

FIG. 20 details a strategy for promoter replacement in front of native mitochondrial transporter sequences using genome editing and a homologous directed repair mechanism. Guide #1 and Guide #2 are used to excise the promoter to be replaced (Promoter 1). A new promoter cassette (Promoter 2), flanked by sequences with homology to the upstream and downstream region of Promoter 1, is introduced and is inserted into the site previously occupied by Promoter 1 using the homologous directed repair mechanism.

### DETAILED DESCRIPTION OF THE INVENTION

Land plants having increased expression of mitochondrial metabolite transporter genes are disclosed. The increased expression of the mitochondrial metabolite transporter genes can result in increased expression of corresponding mitochondrial metabolite transporter proteins. The land plants have increased expression of mitochondrial metabolite transporter genes and/or proteins such that the flux of metabolites through the mitochondrial membrane is increased resulting in increased crop performance and/or yield. The genes encoding the mitochondrial metabolite transporter genes can be used alone or in combinations. The expression of the genes encoding the mitochondrial metabolite transporter proteins can be increased using genetic engineering techniques or marker assisted breeding approaches to develop plants with increased performance and/or yield. Where genetic engineering techniques are used to increase the expression of the mitochondrial metabolite transporter proteins, the increased expression can be accomplished using transgenic technologies with transporter genes from a source other than the plant being modified, by cis-genic approaches, by introducing additional copies of transporter genes from the same plant species or by genome editing approaches to increase the expression of the transporter genes in a constitutive or seed specific manner. The mitochondrial transporters described herein can be used alone or in combinations with the CCP1 like mitochondrial transporters from algal or plant sources which have been shown to reduce photorespiration/respiration and increase crop yield (e.g. WO 2015/103074, PCT/US2017/016421, and U.S. Provisional Patent Applications 62/462,074 and 62/520,785).

Without wishing to be bound by theory, it is believed, based on the metabolic flux models described in Example 1, that by modifying a land plant to have increased expression of mitochondrial metabolite transporter gene(s) and hence increased flux of metabolites through the mitochondrial membrane, that plants having increased performance and/or yield can be produced. It is clear from stoichiometric modeling (flux-balance analysis) that transport of malate and oxaloacetate across the mitochondrial membrane is an important function under diverse circumstances. Because oxaloacetate can be reduced to malate with NAD(P)H as a cofactor, the malate/oxaloacetate pair serves as a surrogate for transfer of reducing equivalents into or out of the mitochondrion. The directionality depends upon the feedstock, the end products, and the amount of light, as all of these factors affect the production and consumption of NAD(P)H and ATP. In some cases it may be beneficial to remove excess reducing equivalents from the mitochondrion, such as during photorespiration, when the conversion of glycine to serine in the mitochondrion generates NADH. In other cases it may be beneficial to achieve a net import of reducing equivalents into the mitochondrion, such as under conditions where respiration is required for sufficient ATP generation. It can be advantageous to import reducing equivalents in this way rather than utilizing the TCA cycle, which generates CO₂ and can therefore undermine net carbon fixation. By increasing the flux of metabolites through the mitochondrial membrane, we believe that the plant can respond better to changing growth conditions, reducing the impact of metabolic feedback loops and making the plant overall more efficient.

In some examples, the land plants with increased expression of mitochondrial metabolite transporter genes also have increased expression of plastidial dicarboxylate transporter genes. The increased expression of the plastidial dicarboxylate transporter genes can result in increased expression of corresponding plastidial dicarboxylate transporter proteins. Without wishing to be bound by theory, it also is believed that increased expression of mitochondrial metabolite transporter genes can result in increased expression of plastidial dicarboxylate transporter genes, based on the observation that CCP1 expression in Camelina sativa, perhaps by altering the dicarboxylate profile of the cytosol, appears to induce this complementary function in the form of the protein encoded at locus Csa10909s010. We postulate that CCP1 is a dicarboxylate transporter whose primary function is to transport malate and oxaloacetate into and out of the mitochondrion, and that in order for CCP1 to have a beneficial effect on carbon fixation and crop yield, CCP1 would need to be paired with a complementary function that serves to direct malate/oxaloacetate into and out of the chloroplast.

Similarly, land plants with increased expression of plastidial dicarboxylate transporter genes also are disclosed. The land plants comprise increased expression of plastidial dicarboxylate transporter genes such that the flux of metabolites through the plastidial membrane is increased, resulting in increased crop performance and/or yield too. Without wishing to be bound by theory, it also is believed that by modifying a land plant to have increased expression of plastidial dicarboxylate transporter gene(s) and hence increased flux of metabolites through the plastidial membrane, that plants having increased performance and/or yield also can be produced.

In some examples, the land plants with increased expression of plastidial dicarboxylate transporter genes also have increased expression of mitochondrial transporter genes. Without wishing to be bound by theory, it also is believed that overexpression of plastidial dicarboxylate transporter genes may induce expression of genes encoding complementary mitochondrial transporters.

Mitochondrial Transporter Genes and Proteins

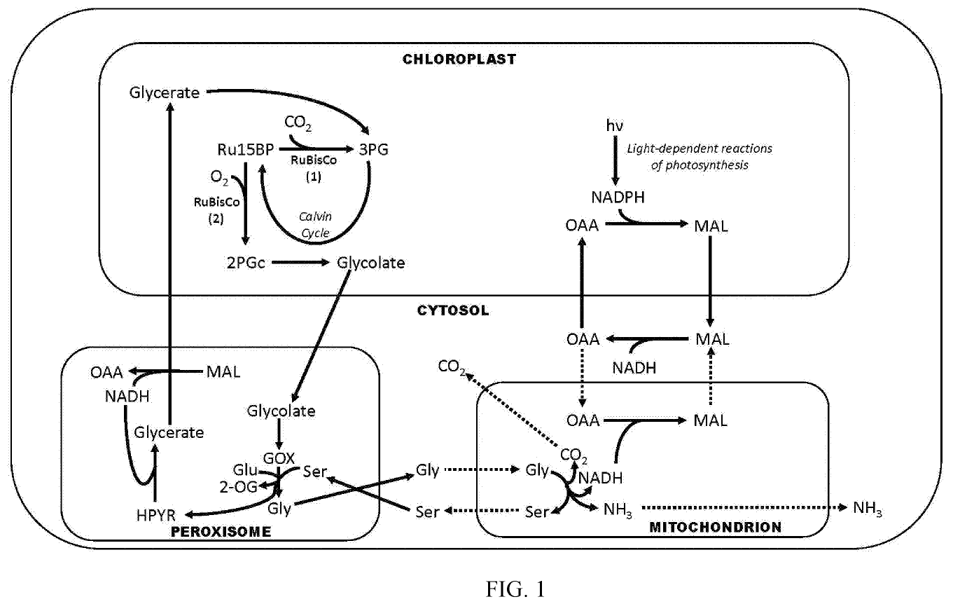

Mitochondrial transporters useful for practicing the disclosed invention include transporters involved in the transport of dicarboxylic acids into and out of the mitochondria in plant cells. In particular these transporters can be involved in the transport of oxaloacetate (OAA) and malate (MAL) as illustrated in FIG. 1. In the case of the transport of OAA and MAL, the transporter can be antiporters such that OAA and MAL are transported simultaneously in the opposite directions, for example such that OAA is transported in, while MAL is transported out. Basically the mitochondrial transporter acts as a malate/oxaloacetate shuttle. In other cases the shuttle may transport OAA and one or more other dicarboxylic acids or other metabolites. Transporters or shuttles which transport OAA are a preferred embodiment of this invention. The directionality of flow of either metabolite is determined by the growth conditions experienced by the plant at any particular time. One aspect where it is useful to transport OAA into the mitochondria occurs when photorespiration is occurring in a photosynthesizing cell and a key requirement is to rid the mitochondria of NADH generated by the conversion of glycine to serine. The DTC- and DIC-type transporters or carriers described in Example 2 can assist in this function, primarily by importing oxaloacetate and exporting the product of its reduction by NADH, malate. They can accomplish this by direct antiport (as is more likely for DICs) or indirectly by coupling oxaloacetate import and malate export to the import and export of other acids, such as 2-oxoglutarate. In a flux-balance simulation of a C3 cell undergoing photorespiration, DTC and DIC can serve parallel functions, and the theoretical yield is the same if either type is knocked out. If both types are knocked out, however, then the theoretical yield does begin to decrease, and mitochondrial NADH is consumed by respiration, whose capacity must increase greatly. Some of the ATP generated by respiration can be exported from the cell by the conversion of glutamate to glutamine by glutamine synthetase. These drastic changes may not be a realistic expectation for the cell and suggest the overall importance of DTC/DIC functions during photorespiration. The DTC/DIC functions are also very important in cells growing heterotrophically or mixotrophically, such as seed cells. Reducing equivalents are produced in these cells by catabolism of sugars delivered through the phloem from photosynthetic cells such as those in leaves, and they can also be produced to some extent by photosynthesis if light reaches the seed cell. This reducing power is used by the mitochondrion for respiration to produce ATP, and a malate (in)/oxaloacetate (out) antiport function, which can be provided by DTC/DIC-type transporters, is an efficient way to deliver reducing equivalents to the mitochondrion for this purpose, especially when they are more plentiful due to photosynthesis. DTC/DIC-type transporters useful for practicing the disclosed invention may be used alone or in combination, for example by developing a plant with increased expression of DTC, developing a plant with increased expression of DIC, or developing plants with increased expression of DTC and DIC.

Mitochondrial transporter genes from Arabidopsis useful for practicing the invention disclosed herein are described in detail in Example 2, including their sequence ID numbers. Orthologs of these transporter genes in major food and feed crop species including soybean, corn, rice, sorghum, potato and Brassica napus are described in Example 5, along with their gene accession numbers. Although mitochondrial transporter genes from any source can be used, it is preferable to use genes from plant sources and more preferable to use genes and DNA sequences from the plant to be genetically engineered to increase expression of the transporter proteins in the mitochondria of the plant cells. Examples of promoters useful for increasing the expression of mitochondrial transporter proteins for specific dicot crops are disclosed in Table 1. Examples of promoters useful for increasing the expression of mitochondrial transporter proteins in specific monocot plants are disclosed in Table 2. For example, one or more of the promoters from soybean (Glycine max) listed in Table 1 may be used to drive the expression of one or more of the soybean mitochondrial transporter genes listed in Table 4. It may also be useful to increase or otherwise alter the expression of one or more mitochondrial transporters in a specific crop using genome editing approaches as described in Example 8.

TABLE 1
Promoters useful for expression of genes in dicots.

| | | Native | |
|---|---|---|---|
| | | organism | |
| Gene/Promoter | Expression | of promoter | Gene ID* |
| Hsp70 | Constitutive | Glycine max | Glyma. |
| | | | 02G093200 |
| | | | (SEQ ID |
| | | | NO: 36) |
| Chlorophyll A/B | Constitutive | Glycine max | Glyma. |
| Binding Protein | | | 08G082900 |
| (Cab5) | | | (SEQ ID |
| | | | NO: 37) |
| Pyruvate phosphate | Constitutive | Glycine max | Glyma. |
| dikinase (PPDK) | | | 06G252400 |
| | | | (SEQ ID |
| | | | NO: 38) |
| Actin | Constitutive | Glycine max | Glyma. |
| | | | 19G147900 |
| | | | (SEQ ID |
| | | | NO: 39) |
| ADP-glucose | Seed specific | Glycine max | Glyma. |
| pyrophosphorylase | | | 04G011900 |
| (AGPase) | | | (SEQ ID |
| | | | NO: 40) |
| Glutelin C (GluC) | Seed specific | Glycine max | Glyma. |
| | | | 03G163500 |
| | | | (SEQ ID |
| | | | NO: 41) |
| β- | Seed specific | Glycine max | Glyma. |
| fructofuranosidase | | | 17G227800 |
| insoluble isoenzyme | | | (SEQ ID |
| 1 (CIN1) | | | NO: 42) |
| MADS-Box | Cob specific | Glycine max | Glyma. |
| | | | 04G257100 |
| | | | (SEQ ID |
| | | | NO: 43) |
| Glycinin | Seed specific | Glycine max | Glyma. |
| (subunit G1) | | | 03G163500 |
| | | | (SEQ ID |
| | | | NO: 44) |
| oleosin | Seed specific | Glycine max | Glyma. |
| isoform A | | | 16G071800 |
| | | | (SEQ ID |
| | | | NO: 45) |
| Hsp70 | Constitutive | Brassica napus | BnaA09g05860D |
| Chlorophyll A/B | Constitutive | Brassica napus | BnaA04g20150D |
| Binding Protein | | | |
| (Cab5) | | | |
| Pyruvate phosphate | Constitutive | Brassica napus | BnaA01g18440D |
| dikinase (PPDK) | | | |
| Actin | Constitutive | Brassica napus | BnaA03g34950D |
| ADP-glucose | Seed specific | Brassica napus | BnaA06g40730D |
| pyrophos- | | | |
| phorylase (AGPase) | | | |
| Glutelin C (GluC) | Seed specific | Brassica napus | BnaA09g50780D |
| β- | Seed specific | Brassica napus | BnaA04g05320D |
| fructofuranosidase | | | |
| insoluble isoenzyme | | | |
| 1 (CIN1) | | | |
| MADS-Box | Cob specific | Brassica napus | BnaA05g02990D |
| Glycinin | Seed specific | Brassica napus | BnaA01g08350D |
| (subunit G1) | | | |
| oleosin isoform A | Seed specific | Brassica napus | BnaC06g12930D |
| 1.7S napin (napA) | Seed specific | Brassica napus | BnaA01g17200D |

*Gene ID includes sequence information for coding regions as well as associated promoters. 5′ UTRs, and 3′ UTRs and are available at Phytozome (see JGI website phytozome.jgi.doe.gov/pz/portal.html).

TABLE 2
Promoters useful for expression of genes in monocots, including maize and rice.

| Gene/Promoter | Expression | Rice* | Maize* |
|---|---|---|---|
| Hsp70 | Constitutive | LOC_Os05g38530 | GRMZM2G 310431 |
| | | (SEQ ID NO: 28) | (SEQ ID NO: 19) |
| Chlorophyll A/B | Constitutive | LOC_Os01g41710 | AC207722.2_FG009 |
| Binding Protein | | (SEQ ID NO: 29) | (SEQ ID NO: 20) |
| (Cab5) | | | GRMZM2G 351977 |
| | | | (SEQ ID NO: 21) |
| Pyruvate phosphate | Constitutive | LOC_Os05g33570 | GRMZM2G 306345 |
| dikinase (PPDK) | | (SEQ ID NO: 30) | (SEQ ID NO: 22) |
| Actin | Constitutive | LOC_Os03g50885 | GRMZM2G 047055 |
| | | (SEQ ID NO: 31) | (SEQ ID NO: 23) |
| Hybrid cab5/ | Constitutive | N/A | SEQ ID NO: 24 |
| hsp70 intron | | | |
| promoter | | | |
| ADP-glucose | Seed | LOC_Os01g44220 | GRMZM2G 429899 |
| pyrophosphorylase | specific | (SEQ ID NO: 32) | (SEQ ID NO: 25) |
| (AGPase) | | | |
| Glutelin C (GluC) | Seed | LOC_Os02g25640 | N/A |
| | specific | (SEQ ID NO: 33) | |
| β-fructofuranosidase | Seed | LOC_Os02g33110 | GRMZM2G 139300 |
| insoluble isoenzyme | specific | (SEQ ID NO: 34) | (SEQ ID NO: 26) |
| 1 (CIN1) | | | |
| MADS-Box | Cob | LOC_Os12g10540 | GRMZM2G 160687 |
| | specific | (SEQ ID NO: 35) | (SEQ ID NO: 27 |

*Gene ID includes sequence information for coding regions as well as associated promoters. 5′ UTRs, and 3′ UTRs and are available at Phytozome (see JGI website phytozome.jgi.doe.gov/pz/portal.html).

Accordingly, disclosed herein is a genetically engineered land plant having increased expression of one or more mitochondrial transporter proteins.

A land plant is a plant belonging to the plant subkingdom Embryophyta, including higher plants, also termed vascular plants, and mosses, liverworts, and hornworts.

The term “land plant” includes mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

Land plants encompass all annual and perennial monocotyledonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Populus, Camelina, Beta, Solanum, and Carthamus. Preferred land plants are those from the following plant families: Amaranthaceae, Asteraceae, Brassicaceae, Carophyllaceae, Chenopodiaceae, Compositae, Cruciferae, Cucurbitaceae, Euphorbiaceae, Fabaceae, Labiatae, Leguminosae, Papilionoideae, Liliaceae, Linaceae, Malvaceae, Poaceae, Rosaceae, Rubiaceae, Saxifragaceae, Scrophulariaceae, Solanaceae, Sterculiaceae, Tetragoniaceae, Theaceae, Umbelliferae.

The land plant can be a monocotyledonous land plant or a dicotyledonous land plant. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus Annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit. Preferred monocotyledonous plants include maize, rice, wheat, sugarcane, sorghum, oats and barley.

Of particular interest are oilseed plants. In oilseed plants of interest the oil is accumulated in the seed and can account for greater than 10%, greater than 15%, greater than 18%, greater than 25%, greater than 35%, greater than 50% by weight of the weight of dry seed. Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize), and various nut species such as, for example, walnut or almond.

Camelina species, commonly known as false flax, are native to Mediterranean regions of Europe and Asia and seem to be particularly adapted to cold semiarid climate zones (steppes and prairies). The species Camelina sativa was historically cultivated as an oilseed crop to produce vegetable oil and animal feed. In addition to being useful as an industrial oilseed crop, Camelina is a very useful model system for developing new tools and genetically engineered approaches to enhancing the yield of crops in general and for enhancing the yield of seed and seed oil in particular. Demonstrated transgene improvements in Camelina can then be deployed in major oilseed crops including Brassica species including B. napus (canola), B. rapa, B. juncea, B. carinata, crambe, soybean, sunflower, safflower, oil palm, flax, and cotton.

As will be apparent, the land plant can be a C3 photosynthesis plant, i.e. a plant in which RuBisCO catalyzes carboxylation of ribulose-1,5-bisphosphate by use of CO₂ drawn directly from the atmosphere, such as for example, wheat, oat, and barley, among others. The land plant also can be a C4 plant, i.e. a plant in which RuBisCO catalyzes carboxylation of ribulose-1,5-bisphosphate by use of CO₂ shuttled via malate or aspartate from mesophyll cells to bundle sheath cells, such as for example maize, millet, and sorghum, among others.

Accordingly, in some examples the genetically engineered land plant is a C3 plant. Also, in some examples the genetically engineered land plant is a C4 plant. Also, in some examples the genetically engineered land plant is a major food crop plant selected from the group consisting of maize, wheat, oat, barley, soybean, millet, sorghum, potato, pulse, bean, tomato, and rice. In some of these examples, the genetically engineered land plant is maize. Also, in some examples the genetically engineered land plant is a forage crop plant selected from the group consisting of silage corn, hay, and alfalfa. In some of these examples, the genetically engineered land plant is silage corn. Also, in some examples the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

The genetically engineered land plant having increased expression of one or more mitochondrial transporter proteins can have a CO₂ assimilation rate that is higher than for a corresponding reference land plant not having the increased expression. For example, the genetically engineered land plant can have a CO₂ assimilation rate that is at least 5% higher, at least 10% higher, at least 20% higher, or at least 40% higher, than for a corresponding reference land plant that does not have the increased expression.

The genetically engineered land plant having increased expression of one or more mitochondrial transporter proteins also can have a transpiration rate that is lower than for a corresponding reference land plant not having the increased expression. For example, the genetically engineered land plant can have a transpiration rate that is at least 5% lower, at least 10% lower, at least 20% lower, or at least 40% lower, than for a corresponding reference land plant that does not have the increased expression.

The genetically engineered land plant having increased expression of one or more mitochondrial transporter proteins also can have a seed yield that is higher than for a corresponding reference land plant not having the increased the expression. For example, the genetically engineered land plant can have a seed yield that is at least 5% higher, at least 10% higher, at least 20% higher, at least 40% higher, at least 60% higher, or at least 80% higher, than for a corresponding reference land plant that does not have the increased expression.

Following identification of suitable mitochondrial transporter proteins, a genetically engineered land plant having increased expression of the one or more mitochondrial transporter proteins can be made by methods that are known in the art, for example as follows.

DNA constructs useful in the methods described herein include transformation vectors capable of introducing transgenes or other modified nucleic acid sequences into land plants. As used herein, “genetically engineered” refers to an organism in which a nucleic acid fragment containing a heterologous nucleotide sequence has been introduced, or in which the expression of a homologous gene has been modified, for example by genome editing. Transgenes in the genetically engineered organism are preferably stable and inheritable. Heterologous nucleic acid fragments may or may not be integrated into the host genome.

Several plant transformation vector options are available, including those described in Gene Transfer to Plants, 1995, Potrykus et al., eds., Springer-Verlag Berlin Heidelberg New York, Genetically engineered Plants: A Production System for Industrial and Pharmaceutical Proteins, 1996, Owen et al., eds., John Wiley & Sons Ltd. England, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof. See, for example, U.S. Pat. No. 5,639,949.

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. See, for example, U.S. Pat. No. 5,639,949. Alternatively, DNA fragments containing the transgene and the necessary regulatory elements for expression of the transgene can be excised from a plasmid and delivered to the plant cell using microprojectile bombardment-mediated methods.

Zinc-finger nucleases (ZFNs) are also useful in that they allow double strand DNA cleavage at specific sites in plant chromosomes such that targeted gene insertion or deletion can be performed (Shukla et al., 2009, Nature 459: 437-441; Townsend et al., 2009, Nature 459: 442-445).

The CRISPR/Cas9 system (Sander, J. D. and Joung, J. K., Nature Biotechnology, published online Mar. 2, 2014; doi; 10.1038/nbt.2842) is particularly useful for editing plant genomes to modulate the expression of homologous genes encoding enzymes. All that is required to achieve a CRISPR/Cas edit is a Cas enzyme, or other CRISPR nuclease (Murugan et al. Mol Cell 2017, 68:15), and a single guide RNA (sgRNA) as reviewed extensively by others (Belhag et al. Curr Opin Biotech 2015, 32: 76; Khandagale and Nadaf, Plant Biotechnol Rep 2016, 10:327). Several examples of the use of this technology to edit the genomes of plants have now been reported (Belhaj et al. Plant Methods 2013, 9:39; Zhang et al. Journal of Genetics and Genomics 2016, 43: 251).

TALENs (transcriptional activator-like effector nucleases) or meganucleases can also be used for plant genome editing (Malzahn et al., Cell Biosci, 2017, 7:21).

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998)(soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun for Agrisoma technology are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128), Methods for plant regeneration from protoplasts have also been described [Evans et al., in Handbook of Plant Cell Culture, Vol 1, (Macmillan Publishing Co., New York, 1983); Vasil, IK in Cell Culture and Somatic Cell Genetics (Academic, Orlando, 1984)].

Recombinase technologies which are useful for producing the disclosed genetically engineered plants include the cre-lox, FLP/FRT and Gin systems. Methods by which these technologies can be used for the purpose described herein are described for example in (U.S. Pat. No. 5,527,695; Dale and Ow, 1991, Proc. Natl. Acad. Sci. USA 88: 10558-10562; Medberry et al., 1995, Nucleic Acids Res. 23: 485-490).

Transformation protocols as well as protocols for introducing nucleotide sequences into plants may vary depending on the type of plant or plant cell, i.e., monocot or dicot, targeted for transformation.

Suitable methods of introducing nucleotide sequences into plant cells and subsequent insertion into the plant genome are described in US 2010/0229256 A1 to Somleva & Ali and US 2012/0060413 to Somleva et al.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain genetically engineered plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant J. 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Genetically engineered Res. 9: 471-476). Other plants that have successfully been transformed by in planta methods include rapeseed and radish (vacuum infiltration, Ian and Hong, 2001, Genetically engineered Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

The cells that have been transformed may be grown into plants in accordance with conventional techniques. See, for example, McCormick et al. Plant Cell Reports 5:81-84 (1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Genetically engineered plants can be produced using conventional techniques to express any genes of interest in plants or plant cells (Methods in Molecular Biology, 2005, vol. 286, Genetically engineered Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Tissue-preferred promoters include those described by Van Ex et al., 2009, Plant Cell Rep. 28: 1509-1520; Yamamoto et al., 1997, Plant J. 12: 255-265; Kawamata et al., 1997, Plant Cell Physiol. 38: 792-803; Hansen et al., 1997, Mol. Gen. Genet. 254: 337-343; Russell et al., 199), Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Seed-specific promoters can be used to target gene expression to seeds in particular. Seed-specific promoters include promoters that are expressed in various tissues within seeds and at various stages of development of seeds. Seed-specific promoters can be absolutely specific to seeds, such that the promoters are only expressed in seeds, or can be expressed preferentially in seeds, e.g. at rates that are higher by 2-fold, 5-fold, 10-fold, or more, in seeds relative to one or more other tissues of a plant, e.g. stems, leaves, and/or roots, among other tissues. Seed-specific promoters include, for example, seed-specific promoters of dicots and seed-specific promoters of monocots, among others. For dicots, seed-specific promoters include, but are not limited to, bean β-phaseolin, napin, β-conglycinin, soybean oleosin 1, Arabidopsis thaliana sucrose synthase, flax conlinin soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, and globulin 1.

Chemical-regulated promoters can be used to modulate the expression of a gene in a plant through the application of an exogenous chemical regulator.

Specific exemplary promoters useful for expression of genes in dicots and monocots are provided in Table 1 and Table 2, respectively.

Certain embodiments use genetically engineered plants or plant cells having multi-gene expression constructs harboring more than one transgene and promoter. The promoters can be the same or different.

Any of the described promoters can be used to control the expression of one or more of genes, their homologs and/or orthologs as well as any other genes of interest in a defined spatiotemporal manner.

Nucleic acid sequences intended for expression in genetically engineered plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

The coding sequence of the selected gene may be genetically engineered by altering the coding sequence for optimal expression in the crop species of interest. Methods for modifying coding sequences to achieve optimal expression in a particular crop species are well known (Perlak et al., 1991, Proc. Natl. Acad. Sci. USA 88: 3324 and Koziel et al., 1993, Biotechnology 11: 194-200).

Individual plants within a population of genetically engineered plants that express a recombinant gene(s) may have different levels of gene expression. The variable gene expression is due to multiple factors including multiple copies of the recombinant gene, chromatin effects, and gene suppression. Accordingly, a phenotype of the genetically engineered plant may be measured as a percentage of individual plants within a population. The yield of a plant can be measured simply by weighing. The yield of seed from a plant can also be determined by weighing. The increase in seed weight from a plant can be due to a number of factors, including an increase in the number or size of the seed pods, an increase in the number of seed and/or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A genetically engineered plant can be produced by selection of transformed seeds or by selection of transformed plant cells and subsequent regeneration.

In some embodiments, the genetically engineered plants are grown (e.g., on soil) and harvested. In some embodiments, above ground tissue is harvested separately from below ground tissue. Suitable above ground tissues include shoots, stems, leaves, flowers, grain, and seed. Exemplary below ground tissues include roots and root hairs. In some embodiments, whole plants are harvested and the above ground tissue is subsequently separated from the below ground tissue.

Genetic constructs may encode a selectable marker to enable selection of transformation events. There are many methods that have been described for the selection of transformed plants (for review see (Miki et al., Journal of Biotechnology, 2004, 107, 193-232) and references incorporated within). Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of genetically engineered plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296). Improved versions of many of the fluorescent proteins have been made for various applications. It will be apparent to those skilled in the art how to use the improved versions of these proteins, including combinations, for selection of transformants.

The plants modified for enhanced yield may have stacked input traits that include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162).

Plastidial Dicarboxylate Transporter Genes and Proteins

Plastidial dicarboxylate transporters useful for practicing the disclosed invention include transporters involved in the transport of dicarboxylic acids into and out of the chloroplasts in plant cells. Like for mitochondrial transporters, the plastidial dicarboxylate transporters can be involved in the transport of oxaloacetate (OAA) and malate (MAL), e.g. as antiporters, acting as a malate/oxaloacetate shuttle. The plastidial dicarboxylate transporters also may transport oxaloacetate and one or more other dicarboxylic acids or other metabolites. Exemplary plastidial dicarboxylate transporters useful for practicing the invention disclosed herein are described in detail in Example 9, including Table 8, which discloses plastidial dicarboxylate transporters of Arabidopsis, and Table 9, which discloses plastidial dicarboxylate transporters of other major food and feed crop species.

### EXAMPLES

### Example 1. Flux-Balance Analysis of Mitochondrial Transport Functions During Photorespiration

Our data suggest that CCP1 increases plant yield by increasing carbon utilization efficiency, and thus it would be most beneficial when CO₂ availability is relatively low. In photosynthetic organisms, and especially in those that lack a carbon-concentrating mechanism, the most significant change in carbon metabolism upon low CO₂ availability is the onset of photorespiration, which involves many compounds in all the major compartments of the cell. Because we know that CCP1 is a mitochondrial transporter, we used a flux-balance analysis (FBA) model to predict what mitochondrial transport functions are likely to become more important during photorespiration for CO₂ assimilation into biomass. The original source for the stoichiometric data for use in the FBA model was the genome-scale AraGEM model of compartmentalized C3 plant metabolism, based on the genome of Arabidopsis thaliana (Cristiana Gomes de Oliveira Dal'Molin et al., 2010, Plant Physiology 152, 579-589). The linear optimization was performed with the Optimization Toolbox of MATLAB (MathWorks, Natick Mass.).

Constraints and Objective Function

The FBA model was run with a basis of 100 input photons and proceeded in two phases. In the first phase, the objective function was maximization of leaf biomass. The leaf biomass equation was taken from the AraGEM model but would apply reasonably well to most plants. In the second phase, the biomass flux found in the first phase was used as a constraint, and the new objective function was the minimization of the sum of all fluxes. The second phase accomplishes two things: 1) it eliminates large futile cycles that often are part of FBA solutions and can cloud their analysis, and 2) it provides the most efficient solution in terms of carbon flow. Carbon input was limited to CO₂ only, and other permitted inputs were water, oxygen, nitrate, hydrogen sulfide, sulfate, and phosphate. The two cases run were with and without photorespiration; that is, designating that RuBisCo reacts with oxygen either 28% of the time (as observed for C3 plants by Zhu et al., 2010, Annu. Rev. Plant Biol. 61:235-61) or 0% of the time. Then the mitochondrial transport fluxes were compared for the two cases to determine those that changed most significantly under photorespiratory conditions.

The Cheung Model and Antiporters

The AraGEM model treats transport events into and out of organelles as independent. That is, it allows metabolites to be transported singly into and out of organelles for simplicity, even though this is not always the case in reality. Therefore the above simulation was subsequently run as described but substituting the mitochondrial transport stoichiometry from the model of Cheung et al. (2013, Plant J. 75:1050-61), which treats transport activities as they are believed to occur in the plant (sometimes as single transport events but most often as antiport events). The maximum biomass yield did not change when the Cheung transporters were used, but the mitochondrial transport events identified as important during photorespiration were of course different. By using both models in this way, we were able to identify important basic transport functions, regardless of whether known transporters carry them out, and also important transport functions that might be carried out by transporters the plant is known to actually possess.

Functions that are Important During Photorespiration

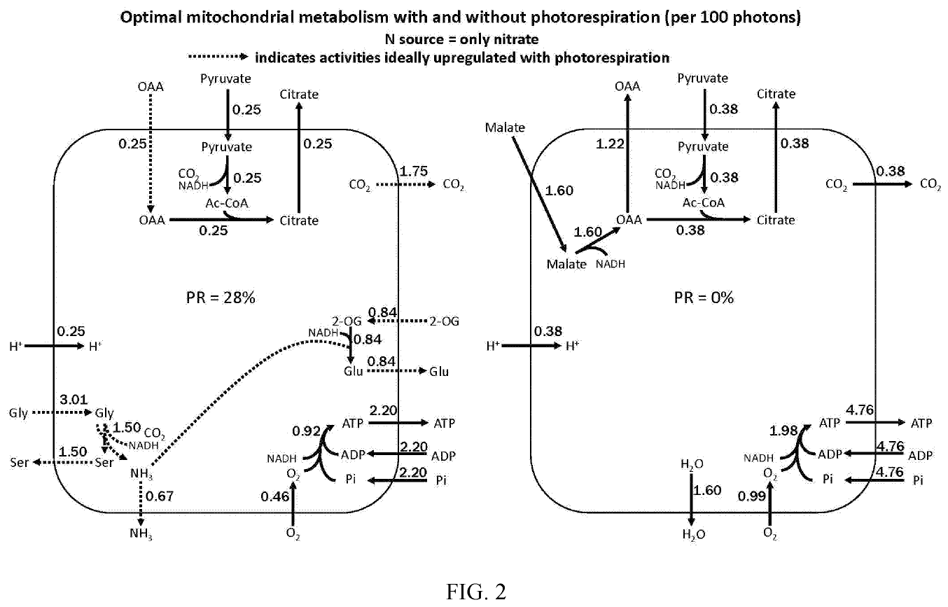

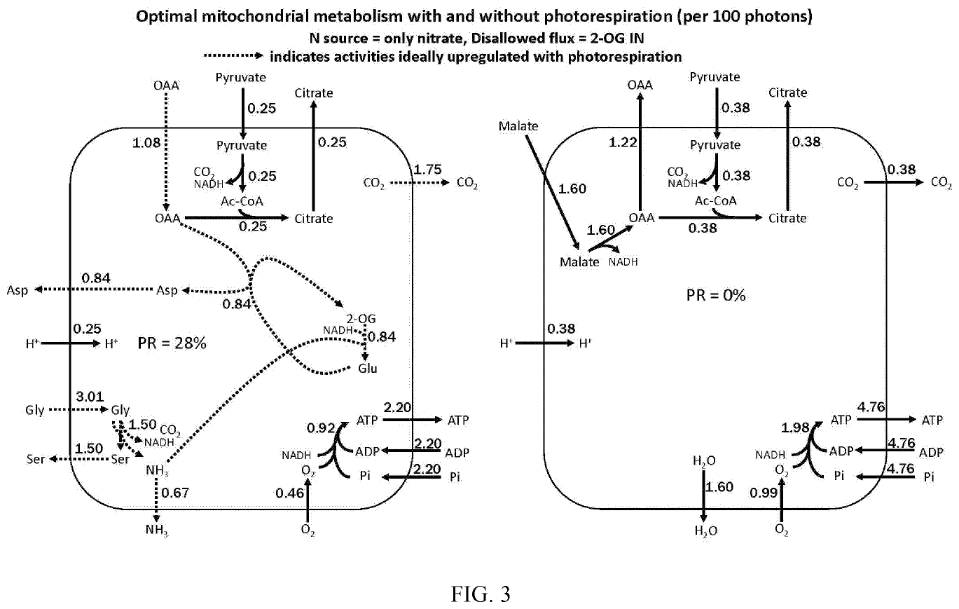

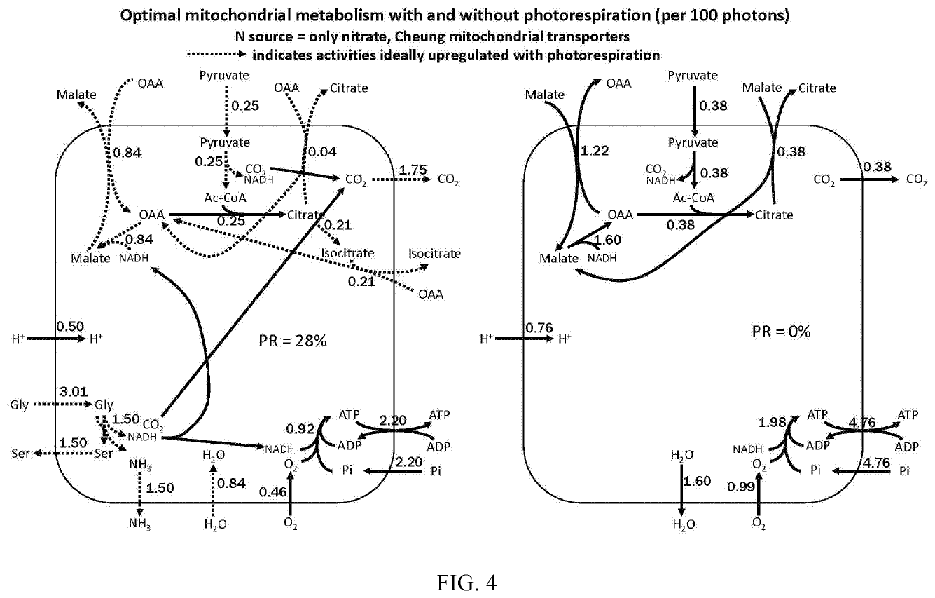

FIG. 2, FIG. 3, and FIG. 4 show the results of the optimizations described above. In FIG. 2 are the results using the AraGEM model and allowing all transport functions. In this case, the transport functions predicted to increase in importance during photorespiration are: glycine import, serine export, ammonia (or ammonium) export, CO₂ (or bicarbonate) export, oxaloacetate import, 2-oxoglutarate import, and glutamate export. The main reason for these functions is the increased activity during photorespiration of mitochondrial glycine hydroxymethyltransferase, which converts glycine to serine. This activity also liberates CO₂, ammonia, and NADH. The most efficient way to deal with this is to use glutamate dehydrogenase, because it consumes both ammonia and NADH. This is why the model identifies 2-oxoglutarate import and glutamate export as important transport activities. Because photorespiration gives rise to so much mitochondrial NADH, the other main transport difference predicted by the model is the elimination of the need to import malate as a source of NADH, followed by oxaloacetate export. In fact, the situation reverses, and oxaloacetate is imported. In FIG. 2, the oxaloacetate import is only carried out as a starting material for citrate synthesis, but in FIG. 3, where 2-oxoglutarate import is disallowed to explore other options for NADH removal, oxaloacetate is imported in much larger quantities as the ultimate acceptor of NADH and ammonia, followed by aspartate export. One can also envision direct acceptance of NADH by oxaloacetate, followed by malate export, although higher independent ammonia export would still be required. In that case, glutamate dehydrogenase would not be required. This is essentially what is shown in FIG. 4, in which the antiporter activities from the Cheung model are used. In this case, the main function of oxaloacetate import is indeed direct acceptance of NADH, although it is also used as a starting material for citrate and isocitrate synthesis. The model that uses the Cheung transporters does not predict the glutamate export option as with the AraGEM model because it has no provision for glutamate export from the mitochondrion.

### Example 2. Transporters Useful for Import of Dicarboxylic Acids and Oxaloacetate in Crop Plants

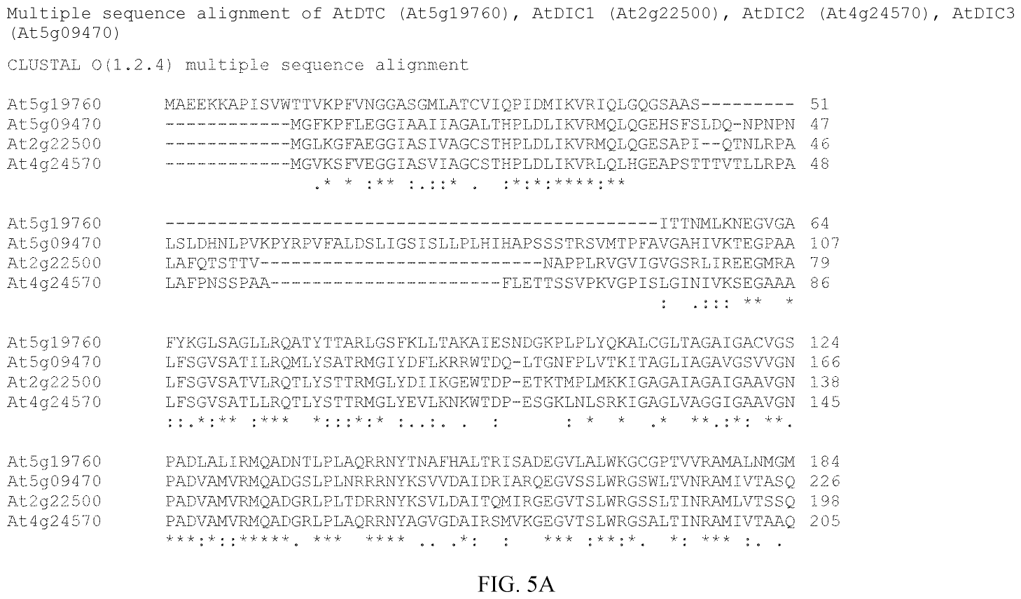

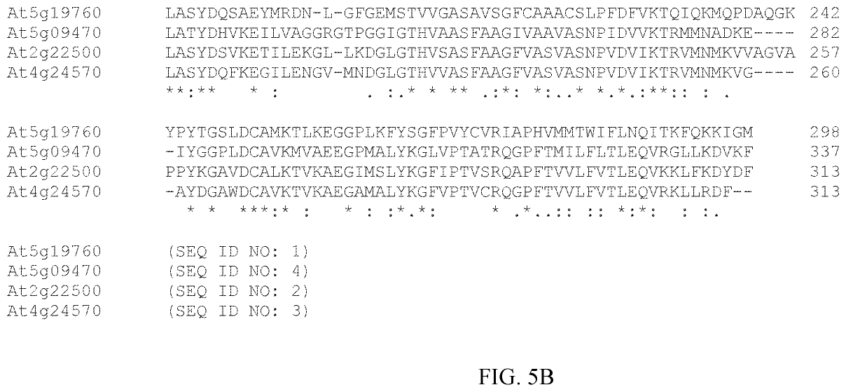

It is instructive to examine how the NADH-removal function via import and export of organic acids might be augmented in an actual plant mitochondrion using transporters the plant already possesses. These kinds of transporters would make desirable gene-editing targets for increasing crop yields in that their regulation could be changed by the insertion of promoters or regulatory elements also derived from the host plant. The Cheung model derives its transport functions from the review of Linka and Weber, 2010, Molec. Plant 3:21-53, which identifies mitochondrial transporters that could be involved in oxaloacetate transport (“dicarboxylate carriers”) as DTC, DIC1, DIC2, and DIC3, found at the Arabidopsis thaliana loci At5g19760 (SEQ ID NO: 1), At2g22500 (SEQ ID NO: 2), At4g24570 (SEQ ID NO: 3), and At5g09470 (SEQ ID NO: 4), respectively. DTC was found to be an antiporter that accepts oxaloacetate as one of its most favored substrates in Arabidopsis (AtDTC) and in tobacco (NtDTC1 and NtDTC3) (Picault et al., 2002, J. Biol. Chem. 277:24204-24211). The isoforms AtDIC1, AtDIC2, and AtDIC3 were found to transport malate, oxaloacetate, succinate, maleate, malonate, phosphate, sulfate and thiosulfate as antiporters. Pastore et al. (2003, Plant Physiol. 133, 2029-2039) showed that the rate of antiport of malate and oxaloacetate determined the overall rate of NADH oxidation by mitochondria in etiolated durum wheat and potato cell suspension culture. This makes more plausible the notion that an antiporter involving oxaloacetate could limit the rate at which the mitochondrion is able to rid itself of excess reducing equivalents generated by photorespiration, as is proposed here. FIG. 5A-B shows a multiple sequence alignment of DTC (SEQ ID NO: 1), DIC1 (SEQ ID NO: 2), DIC2 (SEQ ID NO: 3), and DIC3 (SEQ ID NO: 4) according to CLUSTAL O(1.2.4).

### Example 3. Increased Expression of Transporters in Plants for Increased Mitochondrial Dicarboxylic Acid or Oxaloacetate Transport in Camelina sativa

The transporters DTC (SEQ ID NO: 1), DIC1 (SEQ ID NO: 2), DIC2 (SEQ ID NO: 3), and DIC3 (SEQ ID NO: 4) can be overexpressed in plants by placing the transgene encoding the specific transporter under the control of the appropriate promoter sequence. For seed specific expression, a construct containing the oleosin promoter from soybean is used to express the coding sequence for each gene. For constitutive expression, a construct containing the CaMV35 S-tetramer promoter is used to express the coding sequence for each gene. Constructs expressing the transporter proteins are listed in Table 3.

It will be apparent to those skilled in the art that many different promoters are available for expression in plants. Table 1 lists some of the additional options for use in dicots that can be used as alternate promoters for the vectors described in Table 3.

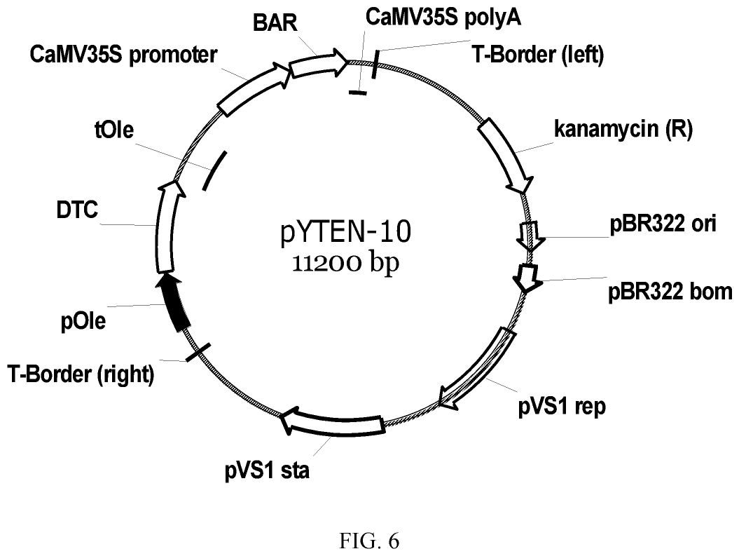

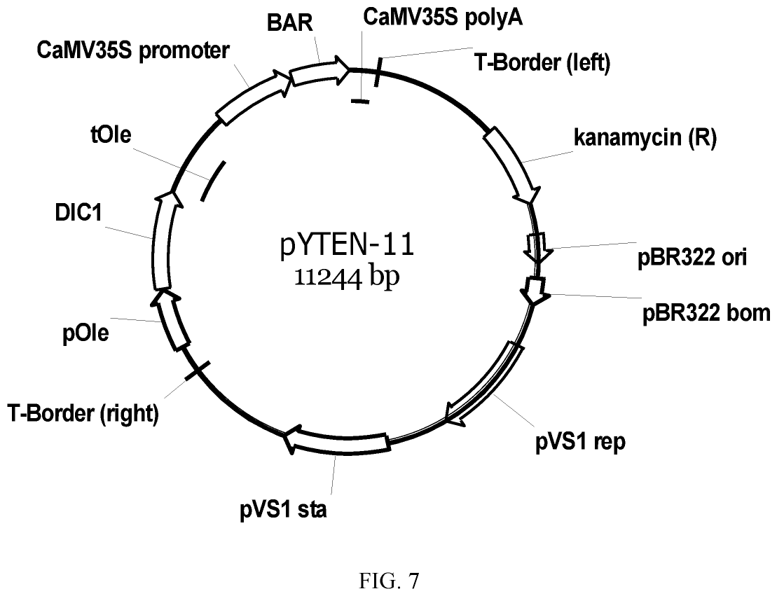

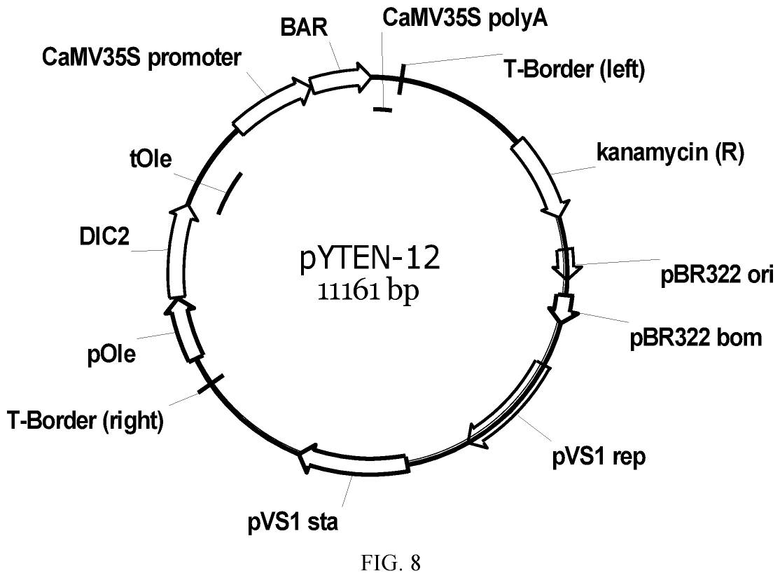

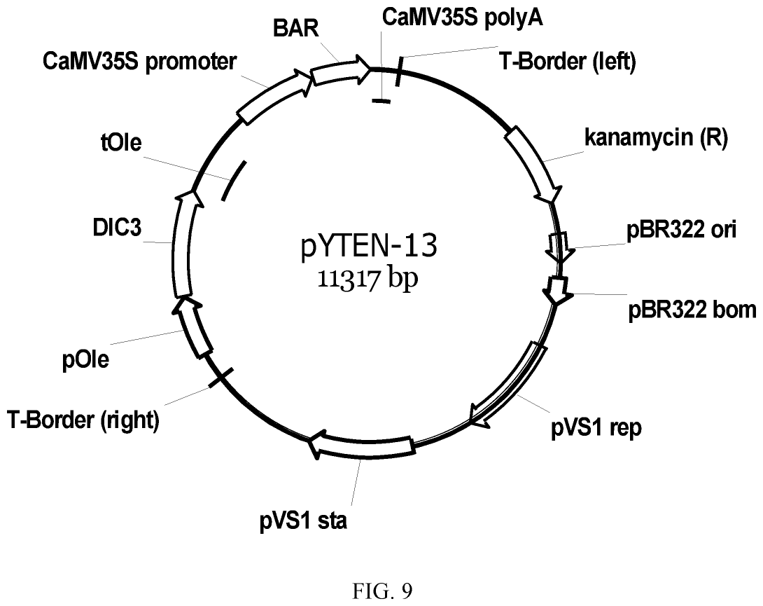

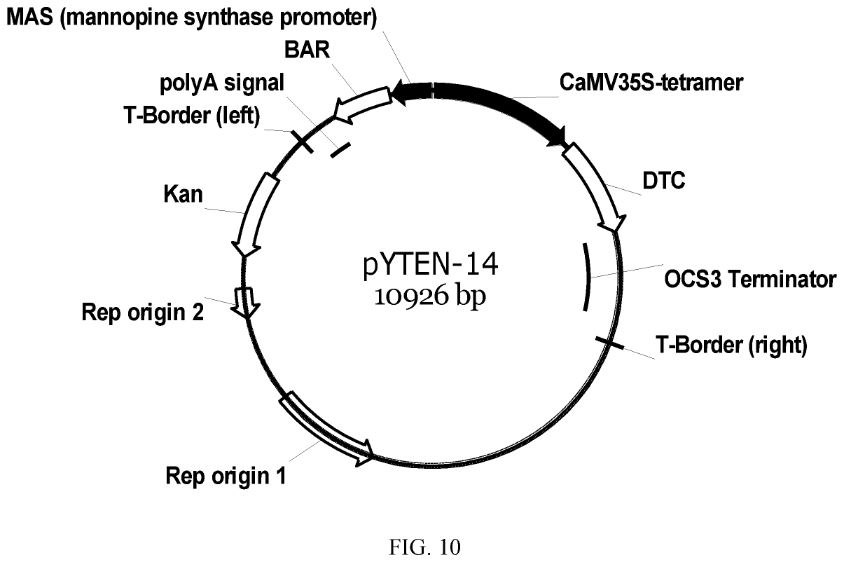

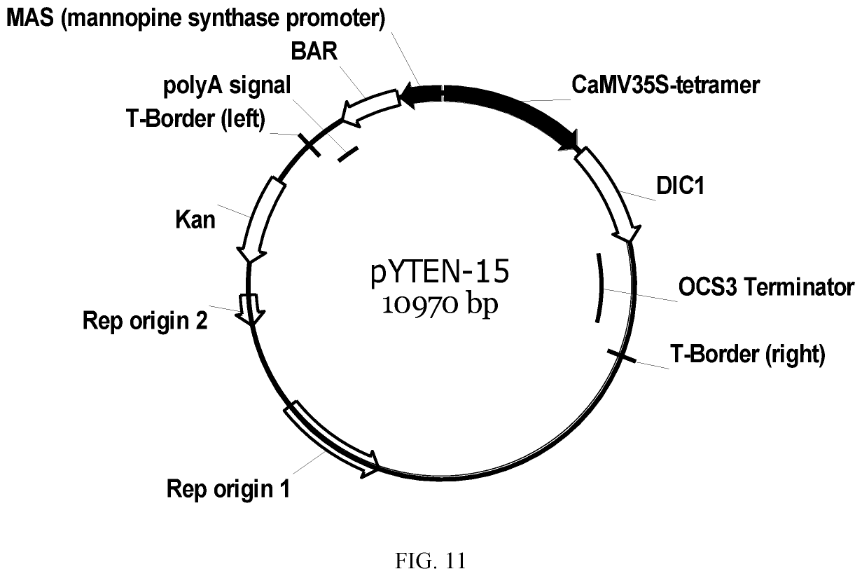

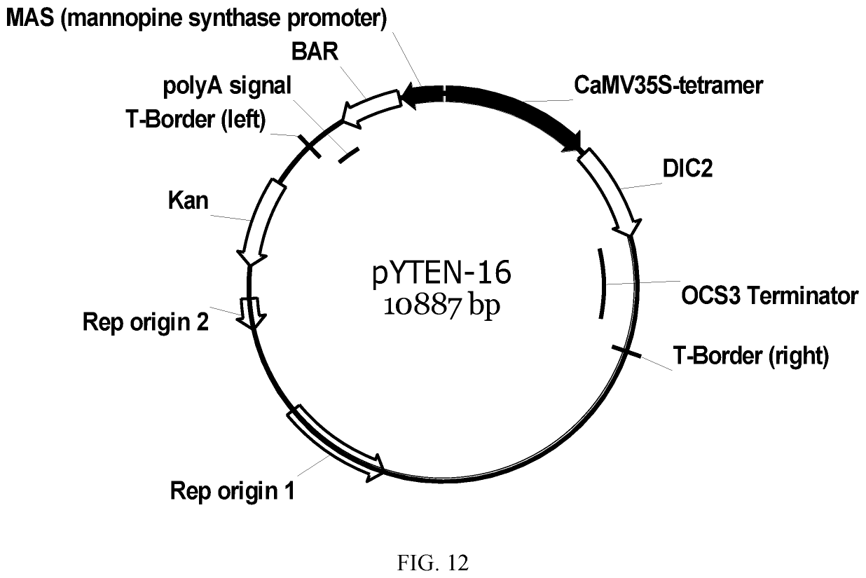

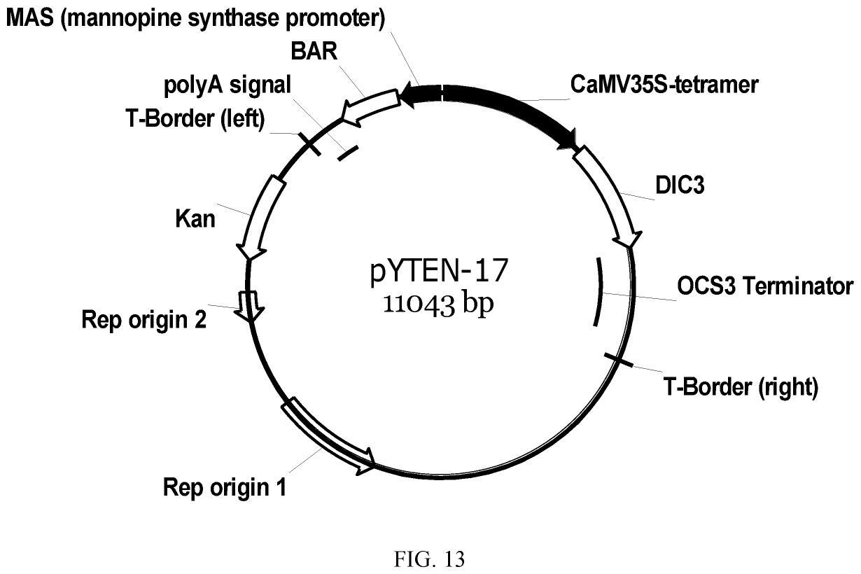

TABLE 3
Constructs for Agrobacterium-mediated transformation of canola
and Camelina for increasing the concentration of mitochondrial
transporters with seed specific or constitutive promoters.

| | | Arabidopsis | | |
|---|---|---|---|---|
| Construct | Transporter | locus; | | SEQ ID/ |
| name | protein | Genbank ID | Promoter | FIG.# |
| pYTEN-10 | DTC | At5g19760; | soybean | SEQ ID NO: |
| | | AY056307 | oleosin | 5 FIG. 6 |
| pYTEN-11 | DIC1 | At2g22500; | soybean | SEQ ID NO: |
| | | AY142648.1 | oleosin | 6 FIG. 7 |
| pYTEN-12 | DIC2 | At4g24570; | soybean | SEQ ID NO: |
| | | AK318852 | oleosin | 7 FIG. 8 |
| pYTEN-13 | DIC3 | At5g09470; | soybean | SEQ ID NO: |
| | | BT033087 | oleosin | 8 FIG. 9 |
| pYTEN-14 | DTC | At5g19760; | CaMV35S- | SEQ ID NO: |
| | | AY056307 | tetramer | 9 FIG. 10 |
| pYTEN-15 | DIC1 | At2g22500; | CaMV35S- | SEQ ID NO: |
| | | AY142648.1 | tetramer | 10 FIG. 11 |
| pYTEN-16 | DIC2 | At4g24570; | CaMV35S- | SEQ ID NO: |
| | | AK318852 | tetramer | 11 FIG. 12 |
| pYTEN-17 | DIC3 | At5g09470; | CaMV35S- | SEQ ID NO: |
| | | BT033087 | tetramer | 12 FIG. 13 |

Constructs can be transformed into Camelina sativa using a floral dip procedure as follows.

In preparation for plant transformation experiments, seeds of Camelina sativa germplasm 10CS0043 (abbreviated WT43, obtained from Agriculture and Agri-Food Canada) are sown directly into 4 inch (10 cm) pots filled with soil in the greenhouse. Growth conditions are maintained at 24° C. during the day and 18° C. during the night. Plants are grown until flowering. Plants with a number of unopened flower buds are used in ‘floral dip’ transformations.

Agrobacterium strain GV3101 (pMP90) is transformed with genetic constructs selected from Table 3 using electroporation. A single colony of GV3101 (pMP90) containing the construct of interest is obtained from a freshly streaked plate and is inoculated into 5 mL LB medium. After overnight growth at 28° C., 2 mL of culture is transferred to a 500-mL flask containing 300 mL of LB and incubated overnight at 28° C. Cells are pelleted by centrifugation (6,000 rpm, 20 min), and diluted to an OD600 of ˜0.8 with infiltration medium containing 5% sucrose and 0.05% (v/v) Silwet-L77 (Lehle Seeds, Round Rock, Tex., USA). Camelina plants are transformed by “floral dip” using the transformation construct of interest as follows. Pots containing plants at the flowering stage are placed inside a 460 mm height vacuum desiccator (Bel-Art, Pequannock, N.J., USA). Inflorescences are immersed into the Agrobacterium inoculum contained in a 500-ml beaker. A vacuum (85 kPa) is applied and held for 5 min. Plants are removed from the desiccator and are covered with plastic bags in the dark for 24 h at room temperature. Plants are removed from the bags and returned to normal growth conditions within the greenhouse for seed formation (T1 generation of seed).

T1 seeds are planted in soil and transgenic plants are selected by spraying a solution of 400 mg/L of the herbicide Liberty (active ingredient 15% glufosinate-ammonium). This allows identification of transgenic plants containing the bar gene on the T-DNA in the plasmid vectors listed in Table 3. Transgenic plant lines are further confirmed using PCR with primers specific to the transporter gene of interest. PCR positive lines are grown in a greenhouse to produce the next generation of seed (T2 seed). Seeds are isolated from each plant and are dried in an oven with mechanical convection set at 22° C. for two days. The weight of the entire harvested seed obtained from individual plants is measured and recorded. The best T2 lines are further propagated in a greenhouse to produce T3 seed. Seeds are isolated from each plant and are dried in an oven with mechanical convection set at 22° C. for two days. The mass of the entire harvested seed obtained from individual plants is measured and recorded and compared to the mass of seeds harvested from wild-type plants grown under the same conditions. The oil content of T3 seeds is measured using published procedures for preparation of fatty acid methyl esters (Malik et al. 2015, Plant Biotechnology Journal, 13, 675-688).

In some instances, it may be advantageous to express the specific transporter from both a seed specific promoter and a constitutive promoter in the same plant to increase the concentration of the transporter protein in the mitochondria. To achieve this, two plasmids, such as pYTEN-10 and pYTEN-14, expressing the DTC protein from seed specific and constitutive promoters, respectively, can separately introduced into Agrobacterium strains, Agrobacterium cultures grown, pelleted, and suspended in infiltration medium as described above. An equal volume of Agrobacterium containing pYTEN-10 and Agrobacterium containing pYTEN-14 are mixed and used for vacuum infiltration. This can be repeated with transformation vectors pYTEN-11 and pYTEN-15 for transporter DIC1, pYTEN-12 and pYTEN-16 for transporter DIC2, and pYTEN-13 and pYTEN-17 for DIC3.

Alternatively, plants expressing individual transporter proteins can be crossed using techniques that are well known to those skilled in the art.

### Example 4. Increased Expression of Transporters in Plants for Increased Mitochondrial Dicarboxylic Acid or Oxaloacetate Transport in Canola

Canola can be transformed with constructs expressing mitochondrial transporter proteins selected from those listed in Table 3 as follows.

In preparation for plant transformation experiments, seeds of Brassica napus cv DH12075 (obtained from Agriculture and Agri-Food Canada) are surface sterilized with sufficient 95% ethanol for 15 seconds, followed by 15 minutes incubation with occasional agitation in full strength Javex (or other commercial bleach, 7.4% sodium hypochlorite) and a drop of wetting agent such as Tween 20. The Javex solution is decanted and 0.025% mercuric chloride with a drop of Tween 20 is added and the seeds are sterilized for another 10 minutes. The seeds are then rinsed three times with sterile distilled water. The sterilized seeds are plated on half strength hormone-free Murashige and Skoog (MS) media (Murashige T, Skoog F (1962). Physiol Plant 15:473-498) with 1% sucrose in 15×60 mm petri dishes that are then placed, with the lid removed, into a larger sterile vessel (Majenta GA7 jars). The cultures are kept at 25° C., with 16 h light/8 h dark, under approx. 70-80 μE of light intensity in a tissue culture cabinet. 4-5 days old seedlings are used to excise fully unfolded cotyledons along with a small segment of the hypocotyl. Excisions are made so as to ensure that no part of the apical meristem is included.

Agrobacterium strain GV3101 (pMP90) carrying the desired mitochondrial transporter protein transformation construct selected from Table 3 is grown overnight in 5 ml of LB media with 50 mg/L kanamycin, gentamycin, and rifampicin. The culture is centrifuged at 2000 g for 10 min., the supernatant is discarded and the pellet is suspended in 5 ml of inoculation medium (Murashige and Skoog with B5 vitamins [MS/B5; Gamborg O L, Miller R A, Ojima K. Exp Cell Res 50:151-158], 3% sucrose, 0.5 mg/L benzyl aminopurine (BA), pH 5.8). Cotyledons are collected in Petri dishes with ˜1 ml of sterile water to keep them from wilting. The water is removed prior to inoculation and explants are inoculated in mixture of 1 part Agrobacterium suspension and 9 parts inoculation medium in a final volume sufficient to bathe the explants. After explants are well exposed to the Agrobacterium solution and inoculated, a pipet is used to remove any extra liquid from the petri dishes.

The Petri plates containing the explants incubated in the inoculation media are sealed and kept in the dark in a tissue culture cabinet set at 25° C. After 2 days the cultures are transferred to 4° C. and incubated in the dark for 3 days. The cotyledons, in batches of 10, are then transferred to selection medium consisting of Murashige Minimal Organics (Sigma), 3% sucrose, 4.5 mg/L BA, 500 mg/L MES, 27.8 mg/L Iron (II) sulfate heptahydrate, pH 5.8, 0.7% Phytagel with 300 mg/L timentin, and 2 mg/L L-phosphinothricin (L-PPT) added after autoclaving. The cultures are kept in a tissue culture cabinet set at 25° C., 16 h/8 h, with a light intensity of about 125 μmol m⁻² s⁻¹. The cotyledons are transferred to fresh selection every 3 weeks until shoots are obtained. The shoots are excised and transferred to shoot elongation media containing MS/B5 media, 2% sucrose, 0.5 mg/L BA, 0.03 mg/L gibberellic acid (GA₃), 500 mg/L 4-morpholineethanesulfonic acid (MES), 150 mg/L phloroglucinol, pH 5.8, 0.9% Phytagar and 300 mg/L timentin and 3 mg/L L-phosphinothricin added after autoclaving. After 3-4 weeks any callus that was formed at the base of shoots with normal morphology is cut off and shoots are transferred to rooting media containing half strength MS/B5 media with 1% sucrose and 0.5 mg/L indole butyric acid, 500 mg/L MES, pH 5.8, 0.8% agar, with 1.5 mg/L L-PPT and 300 mg/L timentin added after autoclaving. The plantlets with healthy shoots are hardened and transferred to 6 inch (15 cm) pots in the greenhouse to collect T1 transgenic seeds.

Screening of transgenic plants of canola expressing transporter proteins to identify plants with higher yield is performed as follows. The T1 seeds of several independent lines are grown in a randomized complete block design in a greenhouse maintained at 24° C. during the day and 18° C. during the night. The T2 generation of seed from each line is harvested. Seed yield from each plant is determined by harvesting all of the mature seeds from a plant and drying them in an oven with mechanical convection set at 22° C. for two days. The weight of the entire harvested seed is recorded. The 100 seed weight is measured to obtain an indication of seed size. The oil content of seeds is measured using published procedures for preparation of fatty acid methyl esters (Malik et al. 2015, Plant Biotechnology Journal, 13, 675-688).

### Example 5. Orthologs of Arabidopsis DTC and DIC1 Transporters in Major Crop Plants

The presence of orthologs of the Arabidopsis DTC (SEQ ID NO: 1), DIC1 (SEQ ID NO: 2), DIC2 (SEQ ID NO: 3), and DIC3 (SEQ ID NO: 4) transporters in major crop plants would allow their modification through cis cloning procedures, where the promoter, transgene, and 3′ UTR are sequences that naturally occur in the plant, or by modification of the expression of the native genes through genome editing. It is favorable to use cis-cloning and genome editing procedures to modify the expression of the transporters since such modifications would have an easier path through regulatory agencies such as USDA-APHIS.

BLAST searches were used to identify orthologs of Arabidopsis DTC and DIC1, abbreviated as AtDTC and AtDIC1, in major crop plants and are shown in Table 4 and Table 5, respectively. In these tables, all Protein BLAST hits with total scores of at least 200 are given, but if no hit attained that score, then the best hit is given.

TABLE 4
Proteins with homology to AtDTC in major crops.

| | | Total | Query | | | |
|---|---|---|---|---|---|---|
| Organism | Description | Score | cover | E value | Identity | Accession |
| Glycine max | mitochondrial dicarboxylate/tricarboxylate | 527 | 99% | 0.0 | 85% | XP_003531254.1 |
| | transporter DTC-like | | | | | |
| | mitochondrial dicarboxylate/tricarboxylate | 527 | 100% | 0.0 | 84% | XP_003524962.1 |
| | transporter DTC | | | | | |
| | unknown | 495 | 91% | 5e−179 | 91% | ACU23390.1 |
| | hypothetical protein GLYMA_05G1578002 | 364 | 69% | 5e−128 | 84% | KRH58947.1 |
| | hypothetical protein GLYMA_05G1578002 | 277 | 51% | 1e−94 | 88% | KRH58948.1 |
| | mitochondrial uncoupling protein 5-like | 209 | 93% | 8e−66 | 38% | XP_003531984.1 |
| | mitochondrial uncoupling protein 4 | 207 | 94% | 3e−65 | 38% | XP_003522752.1 |
| | mitochondrial uncoupling protein 5-like | 204 | 93% | 8e−64 | 40% | XP_003519852.1 |
| | mitochondrial uncoupling protein 5-like | 204 | 93% | 1e−63 | 39% | XP_003517430.1 |
| Zea mays | mitochondrial 2-oxoglutarate/malate carrier | 516 | 96% | 0.0 | 85% | NP_001182793.1 |
| | protein | | | | | |
| | unknown | 516 | 96% | 0.0 | 85% | ACF84711.1 |
| | uncharacterized protein LOC100274318 | 513 | 96% | 0.0 | 85% | NP_001142153.1 |
| | Mitochondrial dicarboxylate/tricarboxylate | 221 | 55% | 1e−72 | 68% | AQK93247.1 |
| | transporter DTC | | | | | |
| Oryza sativa | mitochondrial dicarboxylate/tricarboxylate | 519 | 96% | 0.0 | 85% | XP_015639286.1 |
| Japonica Group | transporter DTC | | | | | |
| | mitochondrial dicarboxylate/tricarboxylate | 508 | 96% | 0.0 | 83% | XP_015615418.1 |
| | transporter DTC | | | | | |
| | hypothetical protein OsJ_17511 | 461 | 86% | 3e−164 | 85% | EEE62708.1 |
| | 2-oxoglutarate/malate translocator | 363 | 73% | 2e−127 | 79% | AAB66888.1 |
| | Os05g0208000 | 345 | 63% | 8e−121 | 63% | BAS92770.1 |
| Triticum aestivum | unnamed protein product | 506 | 96% | 0.0 | 82% | CDM82038.1 |
| Sorghum bicolor | hypothetical protein SORBIDRAFT_09g006480 | 514 | 96% | 0.0 | 85% | XP_002439442.1 |
| Solanum | mitochondrial dicarboxylate/tricarboxylate | 526 | 98% | 0.0 | 85% | NP_001274817.1 |
| tuberosum | transporter DTC-like | | | | | |
| | mitochondrial uncoupling protein 5-like | 220 | 93% | 2e−70 | 40% | XP_006360391.1 |
| | mitochondrial uncoupling protein 5-like | 203 | 93% | 2e−63 | 38% | XP_006353182.1 |
| Brassica napus | mitochondrial dicarboxylate/tricarboxylate | 585 | 100% | 0.0 | 94% | XP_013730718.1 |
| | transporter DTC | | | | | |
| | mitochondrial dicarboxylate/tricarboxylate | 584 | 100% | 0.0 | 94% | XP_013721999.1 |
| | transporter DTC-like | | | | | |
| | mitochondrial dicarboxylate/tricarboxylate | 583 | 100% | 0.0 | 94% | XP_013736363.1 |
| | transporter DTC-like | | | | | |
| | mitochondrial dicarboxylate/tricarboxylate | 583 | 100% | 0.0 | 94% | XP_013676023.1 |
| | transporter DTC | | | | | |
| | mitochondrial dicarboxylate/tricarboxylate | 582 | 100% | 0.0 | 94% | XP_013667347.1 |
| | transporter DTC-like | | | | | |
| | BnaA10g15420D | 580 | 100% | 0.0 | 93% | CDX92503.1 |
| | BnaC03g09720D | 443 | 100% | 2e−158 | 76% | CDX70888.1 |
| | BnaA01g13950D | 211 | 93% | 4e−66 | 39% | CDY34292.1 |
| | mitochondrial uncoupling protein 5-like | 205 | 93% | 7e−64 | 37% | XP_013711831.1 |
| | BnaC08g35020D | 204 | 93% | 1e−63 | 37% | CDX76916.1 |
| | BnaA09g42560D | 201 | 93% | 2e−62 | 36% | CDY13754.1 |

TABLE 5
Proteins with homology to AtDIC1 in major crops.

| | | Total | Query | | | |
|---|---|---|---|---|---|---|
| Organism | Description | Score | cover | E value | Identity | Accession |
| Glycine max | mitochondrial uncoupling protein 5-like | 475 | 99% | 6e−170 | 77% | XP_003519852.1 |
| | mitochondrial uncoupling protein 5-like | 474 | 99% | 1e−169 | 77% | XP_003517430.1 |
| | mitochondrial uncoupling protein 5-like | 464 | 99% | 7e−166 | 72% | XP_003531984.1 |
| | mitochondrial uncoupling protein 4 | 434 | 99% | 4e−154 | 71% | XP_003522752.1 |
| | mitochondrial uncoupling protein 4-like | 233 | 49% | 7e−76 | 73% | XP_006581493.2 |
| | hypothetical protein GLYMA_06G093900 | 215 | 45% | 3e−70 | 74% | KRH52898.1 |
| | mitochondrial uncoupling protein 1-like | 203 | 99% | 4e−63 | 37% | XP_003516932.1 |
| | mitochondrial dicarboxylate/tricarboxylate | 201 | 98% | 2e−62 | 38% | XP_003531254.1 |
| | transporter DTC-like | | | | | |
| Zea mays | mitochondrial 2-oxoglutarate/malate carrier protein | 410 | 100% | 3e−144 | 67% | ONM03746.1 |
| | mitochondrial 2-oxoglutarate/malate carrier protein | 410 | 100% | 5e−144 | 67% | NP_001150641.1 |
| | mitochondrial uncoupling protein 3 | 202 | 97% | 2e−62 | 38% | ACG36575.1 |
| | uncharacterized protein LOC542748 | 201 | 97% | 3e−62 | 37% | NP_001105727.1 |
| Oryza sativa | mitochondrial uncoupling protein 5 | 432 | 100% | 5e−153 | 69% | XP_015650890.1 |
| Japonica Group | | | | | | |
| | 2-oxoglutarate carrier-like protein | 369 | 100% | 9e−128 | 62% | BAD17507.1 |
| | mitochondrial uncoupling protein 5 | 370 | 100% | 4e−127 | 62% | XP_015611796.1 |
| | hypothetical protein OsJ_29672 | 234 | 67% | 5e−75 | 57% | EAZ45034.1 |
| | mitochondrial uncoupling protein 1 | 251 | 97% | 8e−64 | 37% | XP_015616794.1 |
| | uncoupling protein | 247 | 97% | 2e−62 | 37% | BAB40658.1 |
| | mitochondrial carrier protein, putative | 200 | 96% | 6e−62 | 36% | AAX95421.1 |
| Triticum aestivum | unnamed protein product | 195 | 98% | 4e−61 | 37% | CDM82038.1 |
| Sorghum bicolor | hypothetical protein SORBIDRAFT_07g023340 | 409 | 100% | 6e−144 | 67% | XP_002445648.1 |
| | hypothetical protein SORBIDRAFT_05g027910 | 240 | 97% | 8e−62 | 38% | XP_002450079.1 |
| Solanum | mitochondrial uncoupling protein 5-like | 487 | 99% | 4e−175 | 76% | XP_006360391.1 |
| tuberosum | | | | | | |
| | mitochondrial uncoupling protein 5-like | 478 | 99% | 1e−171 | 76% | XP_006353182.1 |
| Brassica napus | BnaC08g35020D | 561 | 100% | 0.0 | 86% | CDX76916.1 |
| | BnaA09g42560D | 557 | 100% | 0.0 | 84% | CDY13754.1 |
| | mitochondrial uncoupling protein 5-like | 549 | 100% | 0.0 | 85% | XP_013711831.1 |
| | mitochondrial uncoupling protein 5 | 543 | 100% | 0.0 | 86% | XP_013743614.1 |
| | mitochondrial uncoupling protein 5-like | 543 | 100% | 0.0 | 86% | XP_013725604.1 |
| | BnaUnng00510D | 480 | 96% | 2e−171 | 79% | CDY27701.1 |
| | BnaA01g13950D | 434 | 99% | 1e−153 | 69% | CDY34292.1 |
| | BnaC01g16430D | 422 | 99% | 6e−149 | 69% | CDY03439.1 |
| | mitochondrial uncoupling protein 4 | 419 | 99% | 1e−147 | 69% | XP_013692904.1 |
| | mitochondrial uncoupling protein 4-like isoform X2 | 419 | 99% | 1e−147 | 69% | XP_013739309.1 |
| | mitochondrial uncoupling protein 4-like isoform X1 | 418 | 99% | 2e−147 | 69% | XP_013739307.1 |
| | mitochondrial uncoupling protein 5-like | 351 | 63% | 2e−122 | 84% | XP_013658861.1 |
| | mitochondrial uncoupling protein 6-like | 345 | 100% | 2e−118 | 58% | XP_013680312.1 |
| | BnaC03g03810D | 345 | 100% | 5e−118 | 57% | CDX81127.1 |
| | mitochondrial uncoupling protein 6 isoform X2 | 343 | 100% | 2e−117 | 57% | XP_013740142.1 |
| | mitochondrial uncoupling protein 6 isoform X1 | 342 | 100% | 4e−117 | 57% | XP_013740141.1 |
| | BnaA03g55840D | 339 | 100% | 5e−116 | 57% | CDY67400.1 |
| | mitochondrial uncoupling protein 1-like | 213 | 98% | 9e−67 | 39% | XP_013707930.1 |
| | mitochondrial uncoupling protein 1 | 209 | 98% | 2e−65 | 39% | XP_013648918.1 |
| | BnaC06g42530D | 209 | 98% | 2e−65 | 39% | CDY51585.1 |
| | mitochondrial uncoupling protein 2 | 205 | 96% | 1e−63 | 39% | XP_013716780.1 |
| | BnaA10g29330D | 206 | 96% | 3e−63 | 40% | CDY55007.1 |
| | mitochondrial uncoupling protein 2-like | 202 | 96% | 2e−62 | 39% | XP_013702150.1 |

### Example 6. Transformation of Maize Orthologs of DTC and DIC1 into Maize Using Biolistics AtDTC Orthologs

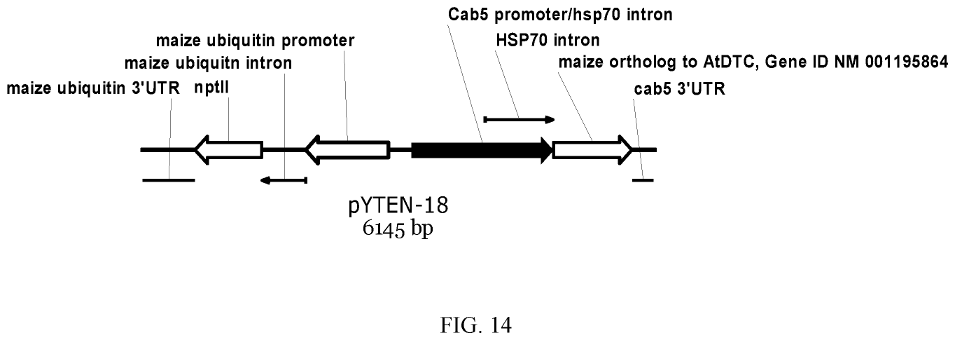

There are multiple orthologs of DTC in maize, including the top four ortholog matches NP_001182793.1, ACF84711.1, NP_001142153.1, and AQK93247.1 listed in Table 4. pYTEN-18 (SEQ ID NO: 13; FIG. 14) is a DNA cassette for biolistic transformation (also known as microparticle bombardment) of monocots such as corn for expression of the maize DTC ortholog NP_001182793.1 (Protein ID), listed as a mitochondrial 2-oxoglutarate/malate carrier protein, using its coding sequence listed in Gene ID NM_001195864.1. It has been designed without the use of plant pest sequences to ease the regulatory path through USDA-APHIS, and extraneous vector backbone material has been removed. USDA-APHIS has previously provided an opinion that maize transformed through biolistic mediated procedures with DNA that does not contain plant pest sequences is not considered a regulated material (website:

www.aphis.usda.gov/biotechnology/downloads/reg_loi/13-242-01_air_response.pdf).

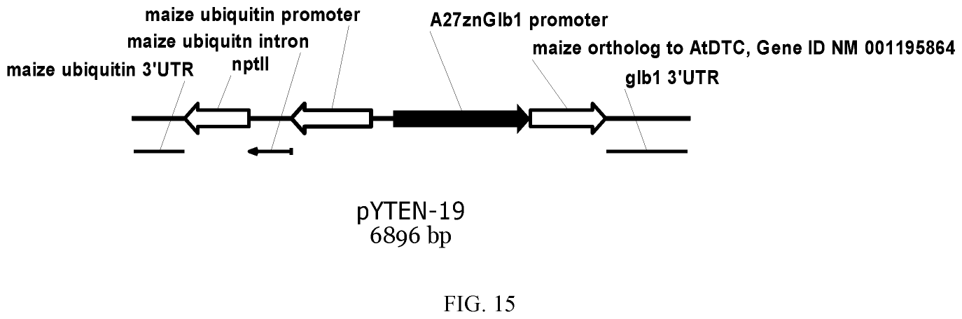

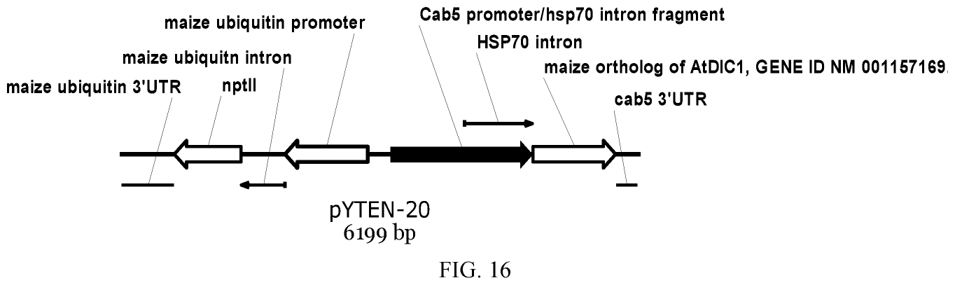

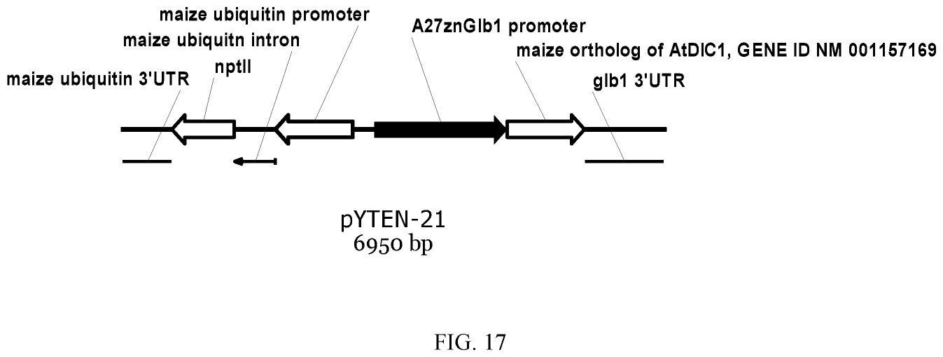

TABLE 6
Constructs for biolistic transformation of maize for increasing
the concentration of maize orthologs of mitochondrial transporters
AtDTC and AtDIC1 with constitutive or seed specific promoters.

| Construct | Ortholog to | | | |
|---|---|---|---|---|
| name | Transporter protein | Protein ID; Gene ID | Promoter | SEQ ID/FIG.# |
| pYTEN-18 | AtDTC | NP_001182793.1; | Cab5/HSP70¹ | SEQ ID NO: 13 |
| | | NM_001195864.1 | | FIG. 14 |
| pYTEN-19 | AtDTC | NP_001182793.1; | Chimeric | SEQ ID NO: 14 |
| | | NM_001195864.1 | A27znGlb1² | FIG. 15 |
| | | | promoter | |
| pYTEN-20 | AtDIC1 | NP_001150641.1; | Cab5/HSP70 | SEQ ID NO: 15 |
| | | NM_001157169.1 | | FIG. 16 |
| pYTEN-21 | AtDIC1 | NP_001150641.1; | A27znGlb1 | SEQ ID NO: 16 |
| | | NM_001157169.1 | promoter | FIG. 17 |

¹Zea mays Cab5 promoter with Zea mays HSP70 intron;
²chimeric promoter consisting of a portion of the promoter from the Zea mays 27 kDa gamma zein gene and a portion of the promoter from the Zea mays globulin-1 gene

### AtDTC Orthologs

In DNA fragment pYTEN-18, the coding sequence for the maize ortholog of AtDTC is expressed from the hybrid maize cab5 promoter containing the maize HSP70 intron. There is an NPTII gene, encoding neomycin phosphotransferase from Escherichia coli K-12, conferring resistance to kanamycin for selection of transformants. The NPTII gene is expressed form the maize ubiquitin promoter with a 3′UTR from the maize ubiquitin gene. DNA fragment pYTEN-18 can be transformed into maize protoplasts, calli, or immature embryos using biolistics as reviewed in Que et al., 2014.

In some cases, it will be advantageous to express the maize orthologs of AtDTC from a seed specific promoter. There are many seed specific promoters known and it will be apparent to those skilled in the art that seed specific promoters from multiple different sources can be used to practice the invention, including the promoters listed in TABLE 2.

DNA fragment pYTEN-19 (SEQ ID NO: 14; FIG. 15) is designed for biolistic transformation of monocots such as corn for expression of the maize DTC ortholog NP_001182793.1 (Protein ID), using its coding sequence listed in Gene ID NM_001195864.1. DNA fragment pYTEN-19 contains the A27znGlb1 chimeric promoter (Accession number EF064989) consisting of a portion of the promoter from the Zea mays 27 kDa gamma zein gene and a portion of the promoter from the Zea mays globulin-1 gene (Shepard & Scott, 2009, Biotechnol. Appl. Biochem., 52, 233-243) controlling the expression of the maize DTC ortholog gene. This promoter has been shown by Shepard and Scott to be active in both the embryo and endosperm of corn kernels. The maize DTC ortholog gene is flanked at the 3′ end by the 3′ UTR, polyA, and terminator from the globulin-1 gene (Accession AH001354.2). It also contains the NPTII gene expressed form the maize ubiquitin promoter with a 3′UTR from the maize ubiquitin gene, for selection of transformants. DNA fragment pYTEN-19 can be transformed into maize protoplasts, calli, or immature embryos using biolistics as reviewed in Que et al, 2014.

### AtDIC1 Orthologs

Similarly, expression cassettes for transformation of the maize ortholog of AtDIC1 can be produced using the hybrid Cab5/HSP70 promoter from maize. There are multiple orthologs of DIC1 in maize, including the top four ortholog matches ONM03746.1, NP_001150641.1, ACG36575.1, and NP_001105727.1 listed in Table 5. pYTEN-20 (SEQ ID NO: 15; FIG. 16) is a DNA cassette for biolistic transformation of monocots such as corn for expression of the maize DIC1 ortholog NP_001150641.1 (Protein ID), listed as a mitochondrial 2-oxoglutarate/malate carrier protein, using its coding sequence listed in Gene ID NM_001157169.1. In DNA fragment pYTEN-20, the coding sequence for the maize ortholog of AtDIC1 is expressed from the hybrid maize cab5 promoter containing the maize HSP70 intron. There is an NPTII gene, encoding neomycin phosphotransferase from Escherichia coli K-12, conferring resistance to kanamycin for selection of transformants. The NPTII gene is expressed form the maize ubiquitin promoter with a 3′UTR from the maize ubiquitin gene. DNA fragment pYTEN-20 can be transformed into maize protoplasts, calli, or immature embryos using biolistics as reviewed in Que et al., 2014.

In some cases, it will be advantageous to express the maize orthologs of AtDIC1 from a seed specific promoter. There are many seed specific promoters known and it will be apparent to those skilled in the art that seed specific promoters from multiple different sources can be used to practice the invention, including the promoters listed in TABLE 2.

DNA fragment pYTEN-21 (SEQ ID NO: 16; FIG. 17) is designed for biolistic transformation of monocots such as corn for expression of the maize AtDIC1 ortholog NP_001150641.1 (Protein ID) using its coding sequence listed in Gene ID NM_001157169.1. DNA fragment pYTEN-21 contains the A27znGlb1 chimeric promoter (Accession number EF064989) consisting of a portion of the promoter from the Zea mays 27 kDa gamma zein gene and a portion of the promoter from the Zea mays globulin-1 gene (Shepard & Scott, 2009, Biotechnol. Appl. Biochem., 52, 233-243) controlling the expression of the maize DTC ortholog gene. This promoter has been shown by Shepard and Scott to be active in both the embryo and endosperm of corn kernels. The maize DIC ortholog gene is flanked at the 3′ end by the 3′ UTR, polyA, and terminator from the globulin-1 gene (Accession AH001354.2). It also contains the NPTII gene expressed form the maize ubiquitin promoter with a 3′UTR from the maize ubiquitin gene, for selection of transformants. DNA fragment pYTEN-21 can be transformed into maize protoplasts, calli, or immature embryos using biolistics as reviewed in Que et al, 2014.

It will be apparent to those skilled in the art that many selectable markers can be used in the maize transformation vectors listed in Table 6 that are not derived from plant pest sequences for selection purposes. These include maize acetolactate synthase/acetohydroxy acid synthase (ALS/AHAS) mutant genes conferring resistance to a range of herbicides from the ALS family of herbicides, including chlorsulfuron and imazethapyr; a 5-enolpyruvoylshikimate-3-phosphate synthase (EPSPS) mutant gene from maize, providing resistance to glyphosate; as well as multiple other selectable markers that are all reviewed in Que et al., 2014 (Que, Q. et al., Front. Plant Sci. 5 Aug. 2014; doi.org/10.3389/fpls.2014.00379). Alternatively, the NPTII expression cassette for the vectors listed in Table 6 can be removed from the main vector and can instead be co-transformed on a separate DNA fragment with the cassette expressing the maize orthologs of AtDTC or AtDIC1. Once transgenic plants are produced, plants can be screened for insertion of the NPTII expression cassette at a separate locus from the expression cassette for the maize ortholog of AtDTC or AtDIC1, such that the NPTII marker can be removed from the plant by segregation.

### Example 7. Increased Expression of Transporters in Plants for Increased Dicarboxylic Acid or Oxaloacetate Transport into the Mitochondria Using Soybean Specific Sequences and Biolistics

There are multiple orthologs of AtDTC in soybean (Table 4) and transformation constructs can be designed for seed specific expression of XP_003531254.1, XP_003524962.1, ACU23390.1, KRH58947.1, KRH58948.1, XP_003531984.1, XP_003522752.1, XP_003519852.1, and XP_003517430.1. This is illustrated with the best ortholog to AtDTC with a protein ID of XP_003531254.1 (Table 7) that is annotated in Genbank as a predicted mitochondrial dicarboxylate/tricarboxylate transporter DTC-like.

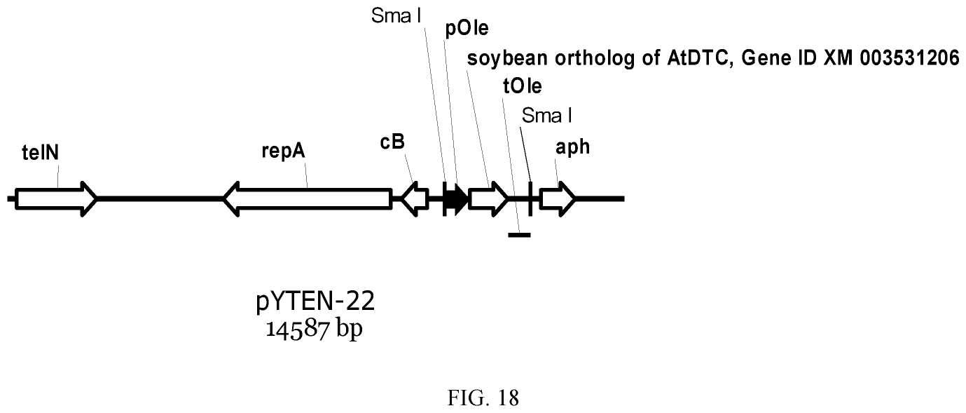

A vector containing the soybean ortholog of AtDTC gene under the control of a seed-specific promoter from the soya bean oleosin isoform A gene is constructed. Plasmid pYTEN-22 (FIG. 18) is a derivative of the pJAZZ linear vector (Lucigen, Inc.) and is constructed using cloning techniques standard for those skilled in the art. The soybean ortholog of AtDTC gene can have its native codon usage or can be codon optimized for expression in soybean. Here the native codon usage of the soybean ortholog of the AtDTC gene is used. The cloning is designed to enable the excision of the soybean ortholog of AtDTC gene expression cassette, using restriction digestion. Digestion of pYTEN-22 with Sma I will release a 2.03 kb cassette containing the expression cassette consisting of the oleosin promoter, the soybean ortholog of AtDTC gene, and oleosin terminator such that no vector backbone will be integrated into the plant.

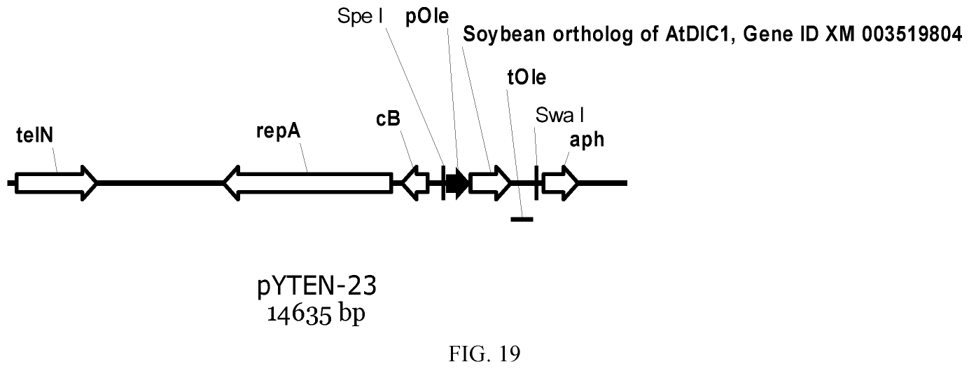

TABLE 7
Constructs for biolistic transformation of soybean for increasing
the concentration of soybean orthologs of mitochondrial transporters
AtDTC and AtDIC1 with seed specific promoters.

| Construct | Ortholog to | | | |
|---|---|---|---|---|
| name | Transporter protein | Protein ID; Gene ID | Promoter | SEQ ID/FIG.# |
| pYTEN-22 | AtDTC | XP_003531254.1; | Soybean | SEQ ID NO: 17 |
| | | XM_003531206 | oleosin | FIG. 18 |
| pYTEN-23 | AtDIC1 | XP_003519852.1; | Soybean | SEQ ID NO: 18 |
| | | XM_003519804 | oleosin | FIG. 19 |

There are multiple orthologs of AtDIC1 gene in soybean (Table 5) and transformation constructs can be designed for seed specific expression of XP_003519852.1, XP_003517430.1, XP_003531984.1, XP_003522752.1, XP_006581493.2, KRH52898.1, XP_003516932.1, and XP_003531254.1. This is illustrated with the best ortholog to AtDIC1 with a protein ID of XP_003519852.1 (Table 7) that is annotated in Genbank as a mitochondrial uncoupling protein 5-like.

A vector containing the soybean ortholog of AtDIC1 gene under the control of a seed-specific promoter from the soya bean oleosin isoform A gene is constructed. Plasmid pYTEN-23 (FIG. 19) is a derivative of the pJAZZ linear vector (Lucigen, Inc.) and was constructed using cloning techniques standard for those skilled in the art. The soybean ortholog of AtDIC1 gene can have its native codon usage or can be codon optimized for expression in soybean. Here the native codon usage of the soybean ortholog of AtDIC1 gene is used. The cloning is designed to enable the excision of the soybean ortholog of AtDIC1 gene expression cassette, using restriction digestion. Digestion of pYTEN-23 with Spe I and Swa I will release a 2.20 kb cassette containing the expression cassette consisting of oleosin promoter, the soybean ortholog of AtDIC1 gene, and oleosin terminator such that no vector backbone will be integrated into the plant.

It will be apparent to those skilled in the art that many different promoters are available for expression in plants. Table 1 lists some of the additional options for use in dicots that can be used as alternate promoters for the vectors described in Table 7.

### Soybean Transformation

The purified fragments for the soybean orthologs of AtDTC and AtDIC1 are transformed with plants. The fragment for the ortholog of AtDTC, isolated from vector pYTEN-22, is co-bombarded with DNA encoding an expression cassette for the hygromycin resistance gene via biolistics into embryogenic cultures of soybean Glycine max cultivars X5 and Westag97, to obtain transgenic plants. The hygromycin resistance gene is expressed from a plant promoter, such as the soybean actin promoter (SEQ ID NO: 39) and the 3′ UTR from the soybean actin gene (soybean actin Gene ID Glyma.19G147900).

The transformation, selection, and plant regeneration protocol is adapted from Simmonds (2003) (Simmonds, 2003, Genetic Transformation of Soybean with Biolistics. In: Jackson J F, Linskens H F (eds) Genetic Transformation of Plants. Springer Verlag, Berlin, pp 159-174) and is performed as follows.

Induction and Maintenance of Proliferative Embryogenic Cultures: Immature pods, containing 3-5 mm long embryos, are harvested from host plants grown at 28/24° C. (day/night), 15-h photoperiod at a light intensity of 300-400 μmol m⁻² s⁻¹. Pods are sterilized for 30 s in 70% ethanol followed by 15 min in 1% sodium hypochlorite [with 1-2 drops of Tween 20 (Sigma, Oakville, ON, Canada)] and three rinses in sterile water. The embryonic axis is excised and explants are cultured with the abaxial surface in contact with the induction medium [MS salts, B5 vitamins (Gamborg O L, Miller R A, Ojima K. Exp Cell Res 50:151-158), 3% sucrose, 0.5 mg/L BA, pH 5.8), 1.25-3.5% glucose (concentration varies with genotype), 20 mg/l 2,4-D, pH 5.7]. The explants, maintained at 20° C. at a 20-h photoperiod under cool white fluorescent lights at 35-75 μmol m⁻² s⁻¹, are sub-cultured four times at 2-week intervals. Embryogenic clusters, observed after 3-8 weeks of culture depending on the genotype, are transferred to 125-ml Erlenmeyer flasks containing 30 ml of embryo proliferation medium containing 5 mM asparagine, 1-2.4% sucrose (concentration is genotype dependent), 10 mg/l 2,4-D, pH 5.0 and cultured as above at 35-60 μmol m⁻² s⁻¹ of light on a rotary shaker at 125 rpm. Embryogenic tissue (30-60 mg) is selected, using an inverted microscope, for subculture every 4-5 weeks.

Transformation: Cultures are bombarded 3 days after subculture. The embryogenic clusters are blotted on sterile Whatman filter paper to remove the liquid medium, placed inside a 10×30-mm Petri dish on a 2×2 cm² tissue holder (PeCap, 1 005 μm pore size, Band SH Thompson and Co. Ltd. Scarborough, ON, Canada) and covered with a second tissue holder that is then gently pressed down to hold the clusters in place. Immediately before the first bombardment, the tissue is air dried in the laminar air flow hood with the Petri dish cover off for no longer than 5 min. The tissue is turned over, dried as before, bombarded on the second side and returned to the culture flask. The bombardment conditions used for the Biolistic PDS-I000/He Particle Delivery System are as follows: 737 mm Hg chamber vacuum pressure, 13 mm distance between rupture disc (Bio-Rad Laboratories Ltd., Mississauga, ON, Canada) and macrocarrier. The first bombardment uses 900 psi rupture discs and a microcarrier flight distance of 8.2 cm, and the second bombardment uses 1100 psi rupture discs and 11.4 cm microcarrier flight distance. DNA precipitation onto 1.0 μm diameter gold particles is carried out as follows: 2.5 μl of 100 ng/μl of insert DNA of pYTEN-22 and 2.5 μl of 100 ng/μl selectable marker DNA (cassette for hygromycin selection) are added to 3 mg gold particles suspended in 50 μl sterile dH₂0 and vortexed for 10 sec; 50 μl of 2.5 M CaCl₂ is added, vortexed for 5 sec, followed by the addition of 20 μl of 0.1 M spermidine which is also vortexed for 5 sec. The gold is then allowed to settle to the bottom of the microfuge tube (5-10 min) and the supernatant fluid is removed. The gold/DNA was resuspended in 200 μl of 100% ethanol, allowed to settle and the supernatant fluid is removed. The ethanol wash is repeated and the supernatant fluid is removed. The sediment is resuspended in 120 μl of 100% ethanol and aliquots of 8 μl are added to each macrocarrier. The gold is resuspended before each aliquot is removed. The macrocarriers are placed under vacuum to ensure complete evaporation of ethanol (about 5 min).

Selection: The bombarded tissue is cultured on embryo proliferation medium described above for 12 days prior to subculture to selection medium (embryo proliferation medium contains 55 mg/l hygromycin added to autoclaved media). The tissue is sub-cultured 5 days later and weekly for the following 9 weeks. Green colonies (putative transgenic events) are transferred to a well containing 1 ml of selection media in a 24-well multi-well plate that is maintained on a flask shaker as above. The media in multi-well dishes is replaced with fresh media every 2 weeks until the colonies are approx. 2-4 mm in diameter with proliferative embryos, at which time they are transferred to 125 ml Erlenmeyer flasks containing 30 ml of selection medium. A portion of the proembryos from transgenic events is harvested to examine gene expression by RT-PCR.

Plant regeneration: Maturation of embryos is carried out, without selection, at conditions described for embryo induction. Embryogenic clusters are cultured on Petri dishes containing maturation medium (MS salts, B5 vitamins, 6% maltose, 0.2% gelrite gellan gum (Sigma), 750 mg/l MgCl₂, pH 5.7) with 0.5% activated charcoal for 5-7 days and without activated charcoal for the following 3 weeks. Embryos (10-15 per event) with apical meristems are selected under a dissection microscope and cultured on a similar medium containing 0.6% phytagar (Gibco, Burlington, ON, Canada) as the solidifying agent, without the additional MgCl₂, for another 2-3 weeks or until the embryos become pale yellow in color. A portion of the embryos from transgenic events after varying times on gelrite are harvested to examine gene expression by RT-PCR.

Mature embryos are desiccated by transferring embryos from each event to empty Petri dish bottoms that are placed inside Magenta boxes (Sigma) containing several layers of sterile Whatman filter paper flooded with sterile water, for 100% relative humidity. The Magenta boxes are covered and maintained in darkness at 20° C. for 5-7 days. The embryos are germinated on solid B5 medium containing 2% sucrose, 0.2% gelrite and 0.075% MgCl₂ in Petri plates, in a chamber at 20° C., 20-h photoperiod under cool white fluorescent lights at 35-75 μmol m⁻² s⁻¹. Germinated embryos with unifoliate or trifoliate leaves are planted in artificial soil (Sunshine Mix No. 3, SunGro Horticulture Inc., Bellevue, Wash., USA), and covered with a transparent plastic lid to maintain high humidity. The flats are placed in a controlled growth cabinet at 26/24° C. (day/night), 18 h photoperiod at a light intensity of 150 μmol m⁻² s⁻¹. At the 2-3 trifoliate stage (2-3 weeks), the plantlets with strong roots are transplanted to pots containing a 3:1:1:1 mix of ASB Original Grower Mix (a peat-based mix from Greenworld, ON, Canada): soil:sand:perlite and grown at 18-h photoperiod at a light intensity of 300-400 μmol m⁻² s⁻¹.

T1 seeds are harvested and planted in soil and grown in a controlled growth cabinet at 26/24° C. (day/night), 18 h photoperiod at a light intensity of 300-400 μmol m⁻² s⁻¹. Plants are grown to maturity and T2 seed is harvested. Seed yield per plant and oil content of the seeds is measured.

The selectable marker can be removed by segregation if desired by identifying co-transformed plants that have not integrated the selectable marker expression cassette and the DTC-like gene cassette into the same locus. Plants are grown, allowed to set seed and germinated. Leaf tissue is harvested from soil grown plants and screened for the presence of the selectable marker cassette. Plants containing only the DTC-like gene expression cassette are advanced.

The above procedure can be repeated for transformation of the fragment containing the expression cassette for the soybean ortholog of AtDIC1, isolated from vector pYTEN-23.

### Example 8. Use of Genome Editing to Alter the Expression of Native Dicarboxylic Acid or Oxaloacetate Transporters in Plants

The expression of the mitochondrial transporters listed in Table 4 and Table 5 can be modified by replacing the native promoter sequences upstream of the transporter coding sequence with a promoter containing a stronger or more optimal tissue specific expression profile. To increase the concentration of the transporter available to the mitochondria, a stronger promoter than the native one is used. The tissue specificity of expression of the promoter can also be modified, to increase or reduce the types of tissues where the gene is expressed.

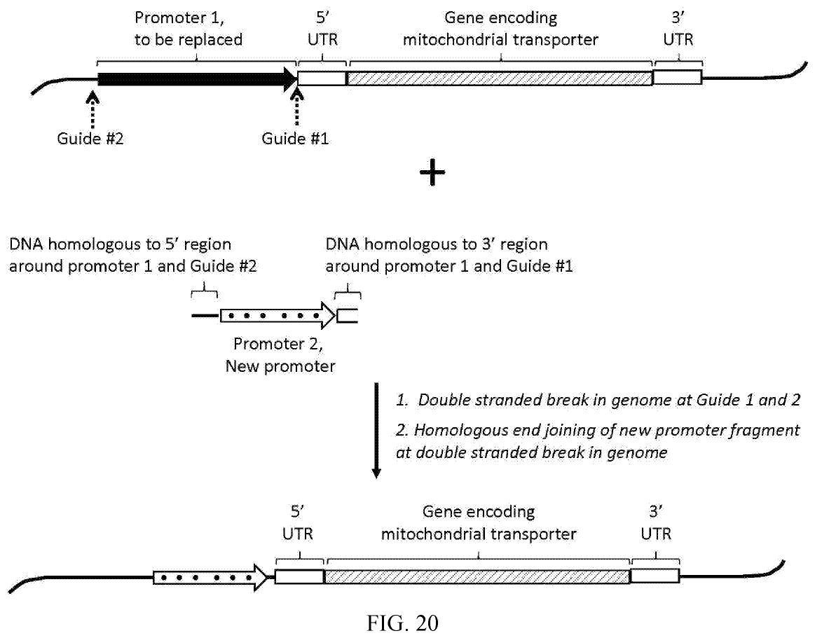

Replacement of the native promoter can be achieved using a genome editing enzyme to make the targeted double stranded cuts to remove the native promoter (Promoter 1) (FIG. 20). The new promoter (Promoter 2) is then inserted via a homology-directed repair (HDR) repair mechanism, in which the new promoter is flanked by DNA sequences with homology to regions upstream and downstream of the original native promoter (Promoter 1).

There are multiple methods to achieve double stranded breaks in genomic DNA, including the use of zinc finger nucleases (ZFN), transcription activator-like effector nucleases (TALENs), engineered meganucleases, and the CRISPR/Cas system (CRISPR is an acronym for clustered, regularly interspaced, short, palindromic repeats and Cas an abbreviation for CRISPR-associated protein) (for review see Khandagal & Nadal, Plant Biotechnol Rep, 2016, 10, 327). CRISPR/Cas mediated genome editing is easiest of the group to implement since all that is needed is the Cas9 enzyme and a short single guide RNA (sgRNA, ˜20 bp) with homology to the modification target to direct the Cas9 enzyme to desired cut site for cleavage. The other methods require more complex design and protein engineering to implement to bind the DNA sequence to enable editing. For this reason, the CRISPR/Cas mediated system has become the method of choice for genome editing.

It will be apparent to those skilled in the art that any of these systems can be used for generating the double stranded breaks necessary for promoter excision in this example.

In this example the CRISPR/Cas system is used. There are many variations of the CRISPR/Cas system that can be used for this technology including the use of wild-type Cas9 from Streptococcus pyogenes (Type II Cas) (Barakate & Stephens, 2016, Frontiers in Plant Science, 7, 765; Bortesi & Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187), the use of a Tru-gRNA/Cas9 in which off-target mutations were significantly decreased (Fu et al., 2014, Nature biotechnology, 32, 279; Osakabe et al., 2016, Scientific Reports, 6, 26685; Smith et al., 2016, Genome biology, 17, 1; Zhang et al., 2016, Scientific Reports, 6, 28566), a high specificity Cas9 (mutated S. pyogenes Cas9) with little to no off target activity (Kleinstiver et al., 2016, Nature 529, 490; Slaymaker et al., 2016, Science, 351, 84), the Type I and Type III Cas Systems in which multiple Cas protein need to be expressed to achieve editing (Li et al., 2016, Nucleic acids research, 44:e34; Luo et al., 2015, Nucleic acids research, 43, 674), the Type V Cas system using the Cpfl enzyme (Kim et al., 2016, Nature biotechnology, 34, 863; Toth et al., 2016, Biology Direct, 11, 46; Zetsche et al., 2015, Cell, 163, 759), DNA-guided editing using the NgAgo Agronaute enzyme from Natronobacterium gregoryi that employs guide DNA (Xu et al., 2016, Genome Biology, 17, 186), and the use of a two vector system in which Cas9 and gRNA expression cassettes are carried on separate vectors (Cong et al., 2013, Science, 339, 819).

It will be apparent to those skilled in the art that any of the CRISPR enzymes can be used for generating the double stranded breaks necessary for promoter excision in this example. There is ongoing work to discover new variants of CRISPR enzymes which, when discovered, can also be used to generate the double stranded breaks around the native promoters of the mitochondrial transporter proteins.

In this example, the CRISPR/Cas9 system is used. FIG. 20 details a strategy for promoter replacement in front of native mitochondrial transporter sequences using CRISPR/Cas9 and a homologous directed repair mechanism. Guide #1 and Guide #2 are used to excise the promoter to be replaced (Promoter 1). A new promoter cassette (Promoter 2), flanked by sequences with homology to the upstream and downstream region of Promoter 1, is introduced and is inserted into the site previously occupied by Promoter 1 using the homologous directed repair mechanism.

It will be apparent to those skilled in the art that many different promoters are available for expression in plants. Table 1 and Table 2 list some of the additional options for use in dicots and monocots that can be used as replacement promoters for the genome editing strategy.

### Example 9. Expression of CCP1 in Camelina sativa Highly Induces Expression of Plastidial Dicarboxylate Transporter Csa10909s010

Expression of CCP1 in Camelina sativa highly induces the plastidial dicarboxylate transporter Csa10909s010 (SEQ ID NO: 46) (Zuber, Joshua, “RNAi Mediated Silencing of Cell Wall Invertase Inhibitors to Increase Sucrose Allocation to Sink Tissues in Transgenic Camelina Sativa Engineered with a Carbon Concentrating Mechanism” (2015). Master's Thesis, May 2014. website: scholarworks.umass.edu/masters_theses_2/218). This protein is homologous to the dicarboxylate transport 2.1 protein (pDCT1) and other Arabidopsis thaliana proteins shown in Table 8.

TABLE 8
Arabidopsis thaliana proteins homologous
to Camelina sativa Csa10909s010.

| | Total | Query | | | |
|---|---|---|---|---|---|
| Description | score | cover | E value | Ident | Accession |
| dicarboxylate | 1024 | 100% | 0.0 | 95% | NP_201234.1 |
| transport 2.1 | | | | | |
| (pDCT1, | | | | | |
| AT5G64290) | | | | | |
| (SEQ ID NO: 47) | | | | | |
| dicarboxylate | 739 | 100% | 0.0 | 69% | NP_201233.1 |
| transporter 2.2 | | | | | |
| (pDCT2, | | | | | |
| AT5G64280) | | | | | |
| (SEQ ID NO: 48) | | | | | |
| dicarboxylate | 485 | 95% | 3e−166 | 50% | NP_568283.2 |
| transporter 1 | | | | | |
| (pOMT1, | | | | | |
| AT5G12860) | | | | | |
| (SEQ ID NO: 49) | | | | | |
| 2-oxoglutarate/ | 416 | 73% | 3e−141 | 51% | AAK43871.1 |
| malate | | | | | |
| translocator | | | | | |
| precursor- | | | | | |
| like protein | | | | | |
| (T24H18.30) | | | | | |
| (SEQ ID NO: 50) | | | | | |

CCP1 is postulated by us to be a dicarboxylate transporter whose primary function is to transport malate and oxaloacetate into and out of the mitochondrion. In order for CCP1 to have a beneficial effect on carbon fixation and crop yield, it would need to be paired with a complementary function that serves to direct malate/oxaloacetate into and out of the chloroplast. CCP1 expression in Camelina sativa, perhaps by altering the dicarboxylate profile of the cytosol, appears to induce this complementary function in the form of the protein encoded at locus Csa10909s010. This may be true in other plants as well.

It is also possible that overexpression of plastidial dicarboxylate transporters may induce the complementary mitochondrial transporter, such as a DIC or DTC. Plastidial dicarboxylate transporters from major crops with homology to Camelina sativa Csa10909s010 are shown in Table 9.

TABLE 9
Proteins with homology to Csa10909s010 in major crops.

| | | Total | Query | | | |
|---|---|---|---|---|---|---|
| Organism | Description | Score | cover | E value | Identity | Accession |
| Glycine max | dicarboxylate transporter 2.1, chloroplastic-like | 766 | 100% | 0.0 | 73% | XP_003531538.1 |
| | (SEQ ID NO: 51) | | | | | |
| | dicarboxylate transporter 2.1, chloroplastic-like | 761 | 100% | 0.0 | 73% | XP_003547089.1 |
| | (SEQ ID NO: 52) | | | | | |
| | dicarboxylate transporter 1, chloroplastic | 464 | 95% | 3e−158 | 46% | XP_003537966.1 |
| | (SEQ ID NO: 53) | | | | | |
| | dicarboxylate transporter 1, chloroplastic-like | 464 | 95% | 6e−158 | 47% | XP_003539493.1 |
| | (SEQ ID NO: 54) | | | | | |
| Zea mays | plastidic general dicarboxylate transporter | 775 | 83% | 0.0 | 80% | NP_001104868.2 |
| | (SEQ ID NO: 55) | | | | | |
| | plastidic general dicarboxylate transporter | 748 | 83% | 0.0 | 78% | NP_001104869.1 |
| | (SEQ ID NO: 56) | | | | | |
| | uncharacterized protein LOC542560 | 460 | 83% | 5e−156 | 51% | NP_001105570.1 |
| | (SEQ ID NO: 57) | | | | | |
| Oryza sativa | dicarboxylate transporter 2.1, chloroplastic | 766 | 83% | 0.0 | 82% | XP_015650655.1 |
| Japonica Group | (SEQ ID NO: 58) | | | | | |
| | dicarboxylate transporter 2.1, chloroplastic | 761 | 88% | 0.0 | 77% | XP_015651303.1 |
| | (SEQ ID NO: 59) | | | | | |
| | hypothetical protein OsJ_29704 | 591 | 75% | 0.0 | 73% | EEE69884.1 |
| | (SEQ ID NO: 60) | | | | | |
| | dicarboxylate transporter 1, chloroplastic | 463 | 83% | 9e−158 | 51% | XP_015620646.1 |
| | (SEQ ID NO: 61) | | | | | |
| Triticum aestivum | cDNA, clone: WT005_N15, cultivar: Chinese Spring | 734 | 83% | 0.0 | 76% | AK333182.1 |
| | (SEQ ID NO: 62) | | | | | |
| | cDNA, clone: WT010_G04, cultivar: Chinese Spring | 416 | 83% | 5e−137 | 47% | AK334584.1 |
| | (SEQ ID NO: 63) | | | | | |
| Sorghum bicolor | dicarboxylate transporter 2.1, chloroplastic | 731 | 93% | 0.0 | 71% | XP_002445990.1 |
| | (SEQ ID NO: 64) | | | | | |
| | dicarboxylate transporter 2.1, chloroplastic | 724 | 83% | 0.0 | 80% | XP_002460379.1 |
| | (SEQ ID NO: 65) | | | | | |
| | dicarboxylate transporter 2, chloroplastic | 689 | 86% | 0.0 | 75% | XP_002451514.1 |
| | (SEQ ID NO: 66) | | | | | |
| | dicarboxylate transporter 2.1, chloroplastic | 686 | 83% | 0.0 | 75% | XP_002445989.2 |
| | (SEQ ID NO: 67) | | | | | |
| | dicarboxylate transporter 1, chloroplastic | 463 | 83% | 1e−157 | 51% | XP_002442229.1 |
| | (SEQ ID NO: 68) | | | | | |
| Solanum | dicarboxylate transporter 2.1, chloroplastic-like | 821 | 100% | 0.0 | 75% | XP_006351757.1 |
| tuberosum | (SEQ ID NO: 69) | | | | | |
| | dicarboxylate transporter 2.1, chloroplastic-like | 614 | 83% | 0.0 | 70% | XP_006353199.1 |
| | (SEQ ID NO: 70) | | | | | |
| | dicarboxylate transporter 1, chloroplastic | 473 | 85% | 3e−162 | 51% | XP_006361749.1 |
| | (SEQ ID NO: 71) | | | | | |
| Brassica napus | dicarboxylate transporter 2.1, chloroplastic-like | 978 | 100% | 0.0 | 92% | XP_013661270.1 |
| | (SEQ ID NO: 72) | | | | | |
| | dicarboxylate transporter 2.1, chloroplastic-like | 973 | 100% | 0.0 | 91% | XP_013652782.1 |
| | (SEQ ID NO: 73) | | | | | |
| | dicarboxylate transporter 2.1, chloroplastic | 972 | 100% | 0.0 | 94% | XP_013643169.1 |
| | (SEQ ID NO: 74) | | | | | |
| | dicarboxylate transporter 2.1, chloroplastic-like | 971 | 100% | 0.0 | 94% | XP_013722814.1 |
| | (SEQ ID NO: 75) | | | | | |
| | dicarboxylate transporter 2.1, chloroplastic-like | 781 | 83% | 0.0 | 93% | XP_013722787.1 |
| | (SEQ ID NO: 76) | | | | | |
| | BnaC02g42990D | 833 | 100% | 0.0 | 68% | CDY46791.1 |
| | (SEQ ID NO: 77) | | | | | |
| | dicarboxylate transporter 2.2, chloroplastic | 734 | 100% | 0.0 | 67% | XP_013700978.1 |
| | (SEQ ID NO: 78) | | | | | |
| | dicarboxylate transporter 2.2, chloroplastic-like | 732 | 100% | 0.0 | 67% | XP_013678357.1 |
| | (SEQ ID NO: 79) | | | | | |
| | dicarboxylate transporter 1, chloroplastic | 463 | 83% | 9e−158 | 51% | XP_013667989.1 |
| | (SEQ ID NO: 80) | | | | | |

Furthermore, there are other similar families of plastidial transporters that may also be useful in this capacity. For example, Taniguchi et al. (2004, Plant and Cell Physiology 45:187-200) identify three distinct types of dicarboxylate transporters in C4 plants: 2-oxoglutarate/malate transporter (OMT), general dicarboxylate transporter (DCT) and oxaloacetate transporter (OAT). Specifically these authors describe in Zea mays the presence of four such plastidic proteins: ZmpOMT1, ZmpDCT1, ZmpDCT2, and ZmpDCT3. Different crops will have different combinations and numbers of OMT, DCT, and OAT genes.

Overexpression of native OMT, DCT, and/or OAT proteins in crop species in combination with expression of CCP1 or its homologs could enhance beneficial yield effects when compared to expression of CCP1 alone. In addition, the overexpression of native OMT, DCT, and/or OAT proteins without expression of CCP1 could provide beneficial yield effects in their own right, whether or not their overexpression causes induction of native CCP1-like mitochondrial functions such as DIC or DTC. It may be beneficial to overexpress OMT, DCT, and/or OAT in mesophyll, bundle sheath, or seed cells, as plastidic and mitochondrial dicarboxylate transport is a beneficial function in all of these cell types.

### EXEMPLARY EMBODIMENTS

Embodiment A: A land plant having increased expression of a mitochondrial transporter protein such that the flux of metabolites through the mitochondrial membrane is increased and the land plant has higher performance and/or yield as compared to a reference land plant not having the increased expression of the mitochondrial transporter protein.

Embodiment B: The land plant of embodiment A, wherein the mitochondrial transporter protein increases the flow of dicarboxylic acids through the mitochondrial membrane, resulting in the land plant having higher performance and/or yield.

Embodiment C: The land plant of embodiment A or B, wherein the mitochondrial transporter protein transports oxaloacetate into or out of the mitochondria of the land plant.

Embodiment D: The land plant of embodiment C, wherein the mitochondrial transporter protein is an oxaloacetate shuttle that transports oxaloacetate through the mitochondrial membrane in one direction while simultaneously transporting another metabolite in the other direction.

Embodiment E: The land plant of embodiment D, wherein the second metabolite is another dicarboxylic acid.

Embodiment F: The land plant of embodiment E, wherein the other dicarboxylic acid is selected from one or more of malate, succinate, maleate, or malonate.

Embodiment G: The land plant of embodiment A or B, wherein the mitochondrial transporter protein comprises one or more of Arabidopsis thaliana DTC (SEQ ID NO: 1), DIC1 (SEQ ID NO: 2), DIC2 (SEQ ID NO: 3), or DIC3 (SEQ ID NO: 4).

Embodiment H: The land plant of embodiment A or B, wherein the mitochondrial transporter protein comprises one or more orthologs of DTC in maize.

Embodiment I: The land plant of embodiment A or B, wherein the mitochondrial transporter protein comprises one or more orthologs of DIC1 in maize.

Embodiment J: The land plant of embodiment A or B, wherein the mitochondrial transporter protein comprises one or more orthologs of DTC in soybean.

Embodiment K: The land plant of embodiment A or B, wherein the mitochondrial transporter protein comprises one or more orthologs of DIC1 in soybean.

Embodiment L: The land plant of embodiment A or B, wherein the mitochondrial transporter protein comprises one or more orthologs of DTC in rice, wheat, sorghum, potato, or canola.

Embodiment M: The land plant of embodiment A or B, wherein the mitochondrial transporter protein comprises one or more orthologs of DIC1 in rice, wheat, sorghum, potato, or canola.

Embodiment N: The land plant of any one of embodiments A-M, wherein the land plant is a genetically engineered land plant, and the increased expression of the mitochondrial transporter protein is based on the genetic engineering.

Embodiment O: The land plant of any one of embodiments A-N, wherein the land plant further has increased expression of a plastidial dicarboxylate transporter protein such that the flux of metabolites through the plastidial membrane is increased and the land plant has higher performance and/or yield as compared to a reference land plant not having the increased expression of the plastidial dicarboxylate transporter protein.

Embodiment P: The land plant of embodiment O, wherein the increased expression of the plastidial dicarboxylate transporter protein is induced by the increased expression of the mitochondrial transporter protein.

Embodiment Q: The land plant of embodiment O or P, wherein the plastidial dicarboxylate transporter protein directs malate and/or oxaloacetate into and/or out of the chloroplasts of the land plant.

Embodiment R: The land plant of any one of embodiments O-Q, wherein the plastidial dicarboxylate transporter protein comprises one or more of Camelina sativa Csa10909s010 (SEQ ID NO: 46), a homolog of Camelina sativa Csa10909s010, or an ortholog of Camelina sativa Csa10909s010.

Embodiment 5: The land plant of any one of embodiments O-Q, wherein the plastidial dicarboxylate transporter protein comprises one or more of a 2-oxoglutarate/malate transporter (OMT), a general dicarboxylate transporter (DCT), or an oxaloacetate transporter (OAT).

Embodiment T: A land plant having increased expression of a plastidial dicarboxylate transporter protein such that the flux of metabolites through the plastidial membrane is increased and the land plant has higher performance and/or yield as compared to a reference land plant not having the increased expression of the plastidial dicarboxylate transporter protein.

Embodiment U: The land plant of embodiment T, wherein the land plant further has increased expression of a mitochondrial transporter protein such that the flux of metabolites through the mitochondrial membrane is increased and the land plant has higher performance and/or yield as compared to a reference land plant not having the increased expression of the mitochondrial transporter protein.

Embodiment V: The land plant of embodiment U, wherein the increased expression of the mitochondrial transporter protein is induced by the increased expression of the plastidial dicarboxylate transporter protein.

Embodiment W: The land plant of embodiment T, wherein the plastidial dicarboxylate transporter protein comprises one or more of Camelina sativa Csa10909s010 (SEQ ID NO: 46), a homolog of Camelina sativa Csa10909s010, or an ortholog of Camelina sativa Csa10909s010.

Embodiment X: The land plant of embodiment T, wherein the plastidial dicarboxylate transporter protein comprises one or more of a 2-oxoglutarate/malate transporter (OMT), a general dicarboxylate transporter (DCT), or an oxaloacetate transporter (OAT).

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “YTEN-57727WO-seq-listing_ST25.txt”, created Jun. 17, 2018, file size of 421,888 bytes, is hereby incorporated by reference.

What is claimed is:

1. A land plant having increased expression of a mitochondrial transporter protein such that the flux of metabolites through the mitochondrial membrane is increased and the land plant has higher performance and/or yield as compared to a reference land plant not having the increased expression of the mitochondrial transporter protein.

2. The land plant of claim 1, wherein the mitochondrial transporter protein increases the flow of dicarboxylic acids through the mitochondrial membrane, resulting in the land plant having higher performance and/or yield.

3. The land plant of claim 1, wherein the mitochondrial transporter protein transports oxaloacetate into or out of the mitochondria of the land plant.

4. The land plant of claim 3, wherein the mitochondrial transporter protein is an oxaloacetate shuttle that transports oxaloacetate through the mitochondrial membrane in one direction while simultaneously transporting another metabolite in the other direction.

5. The land plant of claim 4, wherein the second metabolite is another dicarboxylic acid.

6. The land plant of claim 5, wherein the other dicarboxylic acid is selected from one or more of malate, succinate, maleate, or malonate.

7. The land plant of claim 1, wherein the mitochondrial transporter protein comprises one or more of Arabidopsis thaliana DTC (SEQ ID NO: 1), DIC1 (SEQ ID NO: 2), DIC2 (SEQ ID NO: 3), or DIC3 (SEQ ID NO: 4).

8. The land plant of claim 1, wherein the mitochondrial transporter protein comprises one or more orthologs of DTC in maize.

9. The land plant of claim 1, wherein the mitochondrial transporter protein comprises one or more orthologs of DIC1 in maize.

10. The land plant of claim 1, wherein the mitochondrial transporter protein comprises one or more orthologs of DTC in soybean.

11. The land plant of claim 1, wherein the mitochondrial transporter protein comprises one or more orthologs of DIC1 in soybean.

12. The land plant of claim 1, wherein the mitochondrial transporter protein comprises one or more orthologs of DTC in rice, wheat, sorghum, potato, or canola.

13. The land plant of claim 1, wherein the mitochondrial transporter protein comprises one or more orthologs of DIC1 in rice, wheat, sorghum, potato, or canola.

14. The land plant of claim 1, wherein the land plant is a genetically engineered land plant, and the increased expression of the mitochondrial transporter protein is based on the genetic engineering.

15. The land plant of claim 1, wherein the land plant further has increased expression of a plastidial dicarboxylate transporter protein such that the flux of metabolites through the plastidial membrane is increased and the land plant has higher performance and/or yield as compared to a reference land plant not having the increased expression of the plastidial dicarboxylate transporter protein.

16. The land plant of claim 15, wherein the increased expression of the plastidial dicarboxylate transporter protein is induced by the increased expression of the mitochondrial transporter protein.

17. The land plant of claim 15, wherein the plastidial dicarboxylate transporter protein directs malate and/or oxaloacetate into and/or out of the chloroplasts of the land plant.

18. The land plant of claim 15, wherein the plastidial dicarboxylate transporter protein comprises one or more of Camelina sativa Csa10909s010 (SEQ ID NO: 46), a homolog of Camelina sativa Csa10909s010, or an ortholog of Camelina sativa Csa10909s010.

19. The land plant of claim 15, wherein the plastidial dicarboxylate transporter protein comprises one or more of a 2-oxoglutarate/malate transporter (OMT), a general dicarboxylate transporter (DCT), or an oxaloacetate transporter (OAT).

20. A land plant having increased expression of a plastidial dicarboxylate transporter protein such that the flux of metabolites through the plastidial membrane is increased and the land plant has higher performance and/or yield as compared to a reference land plant not having the increased expression of the plastidial dicarboxylate transporter protein.

21. The land plant of claim 20, wherein the land plant further has increased expression of a mitochondrial transporter protein such that the flux of metabolites through the mitochondrial membrane is increased and the land plant has higher performance and/or yield as compared to a reference land plant not having the increased expression of the mitochondrial transporter protein.

22. The land plant of claim 21, wherein the increased expression of the mitochondrial transporter protein is induced by the increased expression of the plastidial dicarboxylate transporter protein.

23. The land plant of claim 20, wherein the plastidial dicarboxylate transporter protein comprises one or more of Camelina sativa Csa10909s010 (SEQ ID NO: 46), a homolog of Camelina sativa Csa10909s010, or an ortholog of Camelina sativa Csa10909s010.

24. The land plant of claim 20, wherein the plastidial dicarboxylate transporter protein comprises one or more of a 2-oxoglutarate/malate transporter (OMT), a general dicarboxylate transporter (DCT), or an oxaloacetate transporter (OAT).
